(19) United States
(12) Patent Application Publication
(10) Pub. No.: US 2001/0053778 A1
(43) Pub. Date: Dec. 20, 2001

(54) Pharmaceutical compositions of glycogen phosphorylase inhibitors

(76) Inventors: Dennis J. Hoover, Mystic, CT (US); Ravi M. Shanker, Groton, CT (US); James A.S. Nightingale, Bend, OR (US); Dwayne T. Friesen, Bend, OR (US); Douglas A. Lorenz, Bend, OR (US)

Correspondence Address: Gregg C. Benson, Pfizer Inc., Patent Department, MS 4159, Eastern Point Road, Groton, CT, 06340, US

(21) Appl. No.: 09/805,828

(22) Filed: Mar. 14, 2001

Related U.S. Application Data
(60) Provisional application No. 60/189,942, filed on Mar. 16, 2000.

(51) Int. Cl. A61K 31/541; A61K 31/5377; A61K 31/53; A61K 31/501; A61K 31/497

(52) U.S. Cl. 514/227.8; 514/252.06; 514/255.05; 514/256; 514/233.5; 514/241; 514/339; 514/415

(57) ABSTRACT

Pharmaceutical compositions comprise a glycogen phosphorylase inhibitor and at least one concentration-enhancing polymer. The composition may be a simple physical mixture of glycogen phosphorylase inhibitor and concentration-enhancing polymer or a dispersion of glycogen phosphorylase inhibitor and polymer.

### BACKGROUND OF THE INVENTION

This invention relates to pharmaceutical compositions containing a glycogen phosphorylase inhibitor (GPI) and at least one concentration-enhancing polymer, and the use of such pharmaceutical compositions to treat diabetes, hyperglycemia, hypercholesterolemia, hypertension, hyperinsulinemias, hyperlipidemia, atherosclerosis and myocardial ischemia in mammals.

In spite of the early discovery of insulin and its subsequent widespread use in the treatment of diabetes, and the later discovery of and use of sulfonylureas (e.g. Chlorpropamide (Pfizer), Glipizide (Pfizer), Tolbutamide (Upjohn), Acetohexamide (E.I. Lilly), Tolazimide (Upjohn)) and biguanides (e.g. Phenformin (Ciba Geigy), Metformin (G. D. Searle)) as oral hypoglycemic agents, the treatment of diabetes remains less than satisfactory. The use of insulin, necessary in about 10% of diabetic patients in which synthetic hypoglycemic agents are not effective (Type 1 diabetes, insulin dependent diabetes mellitus), requires multiple daily doses, usually by self-injection. Determination of the proper dosage of insulin requires frequent estimations of the sugar in urine or blood. The administration of an excess dose of insulin causes hypoglycemia, with effects ranging from mild abnormalities in blood glucose to coma, or even death. Treatment of non-insulin dependent diabetes mellitus (Type 2 diabetes, NIDDM) usually consists of a combination of diet, exercise, oral agents, e.g. sulfonylureas, and in more severe cases, insulin. However, the clinically available hypoglycemics can have other side effects which limit their use. In any event, where one of these agents may fail in an individual case, another may succeed. A continuing need for hypoglycemic agents, which may have fewer side effects or succeed where others fail, is clearly evident.

Hepatic glucose production is an important target for NIDDM therapy. The liver is the major regulator of plasma glucose levels in the post absorptive (fasted) state, and the rate of hepatic glucose production in NIDDM patients is significantly elevated relative to normal individuals. Likewise, in the postprandial (fed) state, where the liver has a proportionately smaller role in the total plasma glucose supply, hepatic glucose production is abnormally high in NIDDM patients.

Glycogenolysis is an important target for interruption of hepatic glucose production. The liver produces glucose by glycogenolysis (breakdown of the glucose polymer glycogen) and gluconeogenesis (synthesis of glucose from 2- and 3-carbon precursors). Several lines of evidence indicate that glycogenolysis may make an important contribution to hepatic glucose output in NIDDM. First, in normal post absorptive man, up to 75% of hepatic glucose production is estimated to result from glycogenolysis. Second, patients having liver glycogen storage diseases, including Hers' disease (glycogen phosphorylase deficiency), display episodic hypoglycemia. These observations suggest that glycogenolysis may be a significant process for hepatic glucose production.

Glycogenolysis is catalyzed in liver, muscle, and brain by tissue-specific isoforms of the enzyme glycogen phosphorylase (GP). This enzyme cleaves the glycogen macromolecule to release glucose-1-phosphate and a new shortened glycogen macromolecule. Several types of GPIs have been reported to date: glucose and glucose analogs [Martin, J. L. et al., Biochemistry 1991, 30, 10101] and caffeine and other purine analogs [Kasvinsky, P. J. et al., J. Biol. Chem. 1978, 253, 3343-3351 and 9102-9106]. These compounds, and GPIs in general, have been postulated to be of potential use for the treatment of NIDDM by decreasing hepatic glucose production and lowering glycemia [Blundell, T. B. et al. Diabetologia 1992, 35, Suppl. 2, 569-576 and Martin et al. Biochemistry 1991, 30, 10101].

Sites at which GPIs have been reported to bind are the active site, the caffeine or purine binding site, and the ATP or nucleotide binding site. Enzyme activity is also controlled by phosphorylation at a single phosphorylation site, Ser 14. Phosphorylation normally causes an increase in GP activity due to a conformational change in the GP enzyme. The features of this conformational change have been identified. See, Sprang et al., Nature 1988, 336, 215-21. The experimentally determined GP:GPI structures reveal that inhibitor binding at any of the three binding sites named above reverses the conformational change in GP that normally occurs upon phosphorylation causing the GP enzyme to adopt the conformation of the “inactive,” unphosphorylated protein.

Several GPIs have been described. See, e.g., Kristiansen et al., U.S. Pat. No. 5,952,363; Lundgren et al., EP 884 050 A1; Kristiansen et al., WO 98/50359; Bols, WO 97/31901; and Lundgren et al., WO 97/09040. Most of these compounds are cyclic amines with various substitutents that generally render them relatively hydrophilic with good water solubility and good potential for absorption. These GPIs, being water soluble, would thus be expected to not have solubility-limited absorption.

A new binding site has been recently discovered, together with new glycogen phosphorylase inhibitors which bind to this new site. See EP 0978279 A1. As used herein and in the claims, this new binding site shall be referred to as the “indole pocket binding site.” Four different types of GPIs have been identified so far that bind to the indole pocket binding site: See WO 96/39385, U.S. Pat. No. 5,952,322, and EP 846464 A2 which disclose GPIs of the first type; WO 96/39384 and EP 832065 A1 which disclose GPIs of the second type; and U.S. Pat. No. 5,998,463 which discloses GPIs of the third type. A fourth type is disclosed herein. In general, these compounds have in common the structural feature of one or more fused ring systems comprising a six-membered aromatic ring and a nitrogen-containing heterocycle. Such fused ring systems can be considered an “indole-like group,” indole itself having the structure:

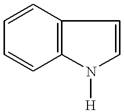

It is believed that GPIs which contain the indole-like group bind to the indole pocket binding site of the GP enzyme. GPIs that bind to this indole pocket binding site generally are relatively hydrophobic, have poor water solubility, and poor bioavailability when dosed conventionally in crystalline form.

Accordingly, what is therefore desired is a composition containing a poorly water soluble GPI that increases the GPI concentration in aqueous solution, does not adversely effect the ability of the GPI to bind to the GP enzyme, improves relative bioavailability, and is pharmaceutically acceptable.

### BRIEF SUMMARY OF INVENTION

The present invention overcomes the aforesaid drawbacks by providing a pharmaceutical composition comprising a glycogen phosphorylase inhibitor and a concentration-enhancing polymer. The GPI binds to a portion or all portions of the following residues of a glycogen phosphorylase enzyme:

| parent | |
|---|---|
| secondary | |
| structure | residue number |
| | 13-23 |
| helix α1 | 24-37 |
| turn | 38-39, 43, 46-47 |
| helix α2 | 48-66, 69-70, 73-74, 76-78 |
| | 79-80 |
| strand β1 | 81-86 |
| | 87-88 |
| strand β2 | 89-92 |
| | 93 |
| helix α3 | 94-102 |
| | 103 |
| helix α4 | 104-115 |
| | 116-117 |
| helix α5 | 118-124 |
| | 125-128 |
| strand β3 | 129-131 |
| | 132-133 |
| helix α6 | 134-150 |
| | 151-152 |
| strand β4 | 153-160 |
| | 161 |
| strand β4b | 162-163 |
| | 164-166 |
| strand β5 | 167-171 |
| | 172-173 |
| strand β6 | 174-178 |
| | 179-190 |
| strand β7 | 191-192 |
| | 194, 197 |
| strand β8 | 198-209 |
| | 210-211 |
| strand β9 | 212-216 |
| strand β10 | 219-226, 228-232 |
| | 233-236 |
| strand β11 | 237-239, 241, 243-247 |
| | 248-260 |
| helix α7 | 261-276 |
| strand β11b | 277-281 |
| reverse turn | 282-289 |
| helix α8 | 290-304. |

In a second aspect of the invention, a pharmaceutical composition comprises a GPI and a concentration-enhancing polymer, the GPI having the general structure of Formula I:

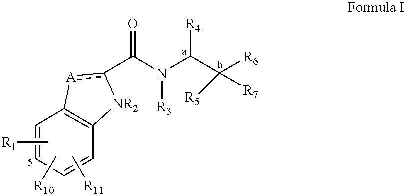

In a third aspect of the invention, a pharmaceutical composition comprises a GPI and a concentration-enhancing polymer, the GPI having the general structure of Formula II:

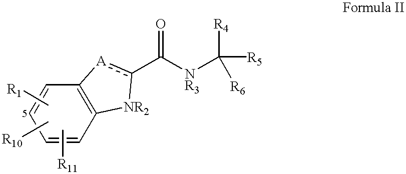

In a fourth aspect of the invention, a pharmaceutical composition comprises a GPI and a concentration-enhancing polymer, the GPI having the general structure of Formula III:

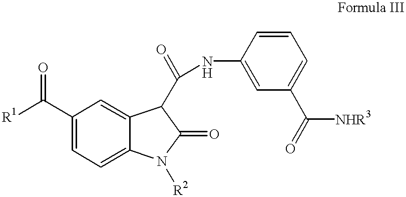

In a fifth aspect of the invention, a pharmaceutical composition comprises a GPI and a concentration-enhancing polymer, the GPI having the general structure of Formula IV:

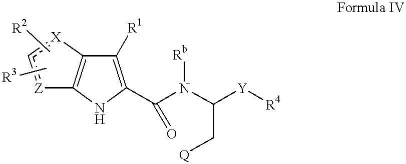

In a sixth aspect of the invention, a pharmaceutical composition comprises a GPI and a concentration-enhancing polymer, the GPI having a solubility in aqueous solution, in the absence of the polymer, of less than 1.0 mg/mL at any pH of from 1 to 8.

In a seventh aspect of the invention, a pharmaceutical composition comprises a GPI and a concentration-enhancing polymer. The composition provides a maximum concentration of the GPI in a use environment that is 1.25-fold that of a control composition comprising an equivalent amount of the GPI and free from the polymer. As used herein, a “use environment” can be either the in vivo environment of the GI tract of an animal, particularly a human, or the in vitro environment of a test solution, such as phosphate buffered saline (PBS) or a Model Fasted Duodenal (MFD) solution.

In an eighth aspect of the invention, a pharmaceutical composition comprises a GPI and a concentration-enhancing polymer. The composition provides a relative bioavailability that is at least 1.25 relative to a control composition comprising an equivalent amount of the GPI and free from the polymer.

In another aspect of the invention, a method of treatment of a mammal having an indication due to atherosclerosis, diabetes, diabetes prevention, diabetic neuropathy, diabetic nephropathy, diabetic retinopathy, cataracts, hypercholesterolemia, hypertriglycerdemia, hypertension, myocardial ischemia, hyperglycemia, hyperinsulimemia, hyperlipidemia, insulin resistance, bacterial infection, tissue ischemia, diabetic cardiomyopathy, or tumor growth inhibition comprises the following steps. A composition of a GPI and a concentration-enhancing polymer is formed. The composition is then administered to the mammal.

The composition may be dosed in a variety of dosage forms, including both initial release and controlled release dosage forms, the latter including both delayed and sustained release forms. The composition may include blends of polymers, and may further include other polymers that improve the aqueous concentration of the GPI. The composition may further comprise other constituents that improve the stability, wetting, dissolution, tableting, or processing characteristics of the composition.

The various aspects of the present invention each have one or more of the following advantages. The compositions increase the concentration of GPI in aqueous solution relative to the crystalline form of the GPI. The compositions also improve relative bioavailability of the GPI. In addition, the compositions enable the use of poorly water soluble, hydrophobic GPIs without adversely affecting their binding characteristics.

The foregoing and other objectives, features, and advantages of the invention will be more readily understood upon consideration of the following detailed description of the invention.

### DETAILED DESCRIPTION OF THE INVENTION

The present invention provides compositions of GPIs and at least one concentration-enhancing polymer. As discussed above in the Background, a new class of poorly water soluble, hydrophobic GPIs has been discovered that bind to the indole pocket binding site in the GP enzyme. It is believed that an important part of the binding of GPIs to this site is due to the indole-like group, which, being relatively hydrophobic, binds in a hydrophobic pocket within the GP enzyme. In studying the GPI activity, binding mode, and GPI/GP complex structure of a wide variety of compounds, it has been found that compounds that have good GP inhibition activity at this indole pocket binding site often have a number of features in common: (1) the presence of one or more indole-like groups in the structure; (2) extremely low solubilities in aqueous solution (i.e., less than 1.0 mg/mL) at physiologically relevant pH (e.g., any pH of from 1 through 8) measured at about 22° C.; (3) a relatively hydrophobic nature; and (4) a relatively low bioavailability when orally dosed in the crystalline state.

Accordingly, unlike other previously known GPIs, GPIs which bind to the indole pocket binding site typically require some kind of modification or formulation to enhance their solubility and thereby achieve good bioavailability. However, the inventors have found that many of the conventional methods used to improve solubility, and in turn bioavailability, have proved problematic. One method used generally to improve drug bioavailability is to form an ionic form of the drug, typically by incorporating an ionizable group into its structure, and particularly by forming a highly soluble salt form. However, the GPIs with the indole-like group having the best performance generally are neutral or nonionic and relatively hydrophobic.

The inventors have found that preparing GPIs having indole-like groups as compositions comprising a GPI and concentration-enhancing polymer, and preferably as a solid dispersion of the GPI and concentration-enhancing polymer, improves the aqueous concentration of the GPIs as well as relative bioavailability, but does not adversely affect the binding characteristics of the GPIs. The compositions, GPIs, suitable polymers, and optional excipients are discussed in more detail as follows.

### COMPOSITIONS OF GPIs AND CONCENTRATION-ENHANCING POLYMER

The present invention finds utility with any low-solubility GPI, or any GPI which would benefit by improved bioavailability. The compositions of the present invention are mixtures comprised of a GPI and at least one concentration-enhancing polymer. The mixtures are preferably solid dispersions, but simple physical mixtures of the GPI and polymer may also be suitable for some GPIs. The GPI in its pure state may be crystalline or amorphous. Preferably, at least a major portion of the GPI in the composition is amorphous. By “amorphous” is meant simply that the GPI is in a non-crystalline state. As used herein, the term “a major portion” of the GPI means that at least 60% of the GPI in the composition is in the amorphous form, rather than the crystalline form. Preferably, the GPI in the composition is substantially amorphous. As used herein, “substantially amorphous” means that the amount of the GPI in crystalline form does not exceed 25%. More preferably, the GPI in the composition is “almost completely amorphous” meaning that the amount of GPI in the crystalline form does not exceed 10%. Amounts of crystalline GPI may be measured by powder X-ray diffraction, Scanning Electron Microscope (SEM) analysis, differential scanning calorimetry (“DSC”), or any other standard quantitative measurement. The composition may contain from about 1 to about 80 wt % GPI, depending on the dose of the GPI. Enhancement of aqueous GPI concentrations and relative bioavailability are typically best at low GPI levels, typically less than about 25 to 40 wt %. However, due to the practical limit of the dosage form size, higher GPI loadings are often preferred and perform well.

In a preferred aspect of the invention, GPI and concentration-enhancing polymer are present as a solid dispersion of the low-solubility GPI and polymer. Preferably, at least a major portion of the GPI in the dispersion is present in the amorphous, rather than the crystalline state. The amorphous GPI can exist as a pure phase, as a solid solution of GPI homogeneously distributed throughout the polymer or any combination of these states or those states that lie intermediate between them.

The dispersion is preferably substantially homogeneous so that the amorphous GPI is dispersed as homogeneously as possible throughout the polymer. As used herein, “substantially homogeneous” means that the GPI present in relatively pure amorphous domains within the solid dispersion is relatively small, on the order of less than 20%, and preferably less than 10% of the total amount of GPI. While the dispersion may have some GPI-rich domains, it is preferred that the dispersion itself have a single glass transition temperature (T_{g}) which demonstrates that the dispersion is substantially homogeneous. This contrasts with a simple physical mixture of pure amorphous GPI particles and pure amorphous polymer particles which generally display two distinct T_{g}s, one that of the GPI and one that of the polymer. T_{g} as used herein is the characteristic temperature where a glassy material, upon gradual heating, undergoes a relatively rapid (e.g., 10 to 100 seconds) physical change from a glass state to a rubber state. Dispersions of the present invention that are substantially homogeneous generally are more physically stable and have improved concentration-enhancing properties and, in turn improved bioavailability, relative to nonhomogeneous dispersions.

While the inventors have found that dispersions of the GPI and concentration-enhancing polymer yield good results, it has been found for at least one GPI that compositions of physical mixtures of amorphous GPI and concentration-enhancing polymer also yield improved aqueous GPI concentration. Al least a major portion of the GPI in the mixture is amorphous. The composition may be in the form of a simple dry physical mixture wherein both the GPI and concentration-enhancing polymer are mixed in particulate form and wherein the particles of each, regardless of size, retain the same individual physical properties that they exhibit in bulk. Any conventional method used to mix the polymer and GPI together such as physical mixing and dry or wet granulation may be used. In this embodiment of the invention, the amorphous GPI and concentration-enhancing polymer need not be directly mixed, but only both present in the dosage form. For example, the amorphous GPI may be in the form of a tablet, bead, or capsule, and the concentration-enhancing polymer may be a coating, granulating material, or even the wall of the capsule.

The compositions comprising the GPI and concentration-enhancing polymer provide enhanced concentration of the GPI in in vitro dissolution tests. It has been determined that enhanced drug concentration in in vitro dissolution tests in Model Fasted Duodenal (MFD solution) or Phosphate Buffered Saline (PBS) is a good indicator of in vivo performance and bioavailability. An appropriate PBS solution is an aqueous solution comprising 20 mM sodium phosphate (Na₂HPO₄), 47 mM potassium phosphate (KH₂PO₄), 87 mM NaCl, and 0.2 mM KCl, adjusted to pH 6.5 with NaOH. An appropriate MFD solution is the same PBS solution wherein additionally is present 14.7 mM sodium taurocholic acid and 2.8 mM of 1-palmitoyl-2-oleyl-sn-glycero-3-phosphocholine. In particular, a composition of the present invention can be dissolution-tested by adding it to MFD or PBS solution and agitating to promote dissolution. Preferably, the composition of the present invention provides a Maximum Drug Concentration (MDC) that is at least 1.25-fold the equilibrium concentration of a control composition comprising an equivalent quantity of GPI but free from the polymer. In other words, if the equilibrium concentration provided by the control composition is 100 μg/mL, then a composition of the present invention provides an MDC of at least 125 μg/mL. The comparison composition is conventionally the undispersed GPI alone (e.g., typically, the crystalline GPI alone in its most thermodynamically stable crystalline form, or in cases where a crystalline form of the GPI is unknown, the control may be the amorphous GPI alone) or the GPI plus a weight of inert diluent equivalent to the weight of polymer in the test composition. More preferably, the MDC of GPI achieved with the compositions of the present invention are at least 2-fold, and even more preferably at least 3-fold, that of the control composition.

Alternatively, the compositions of the present invention provide in an aqueous use environment a concentration versus time Area Under The Curve (AUC), for any period of at least 90 minutes between the time of introduction into the use environment and about 270 minutes following introduction to the use environment that is at least 1.25-fold that of a control composition comprising an equivalent quantity of undispersed GPI.

Alternatively, the dispersion of the present invention, when dosed orally to a human or other animal, provides an AUC in GPI concentration in the blood for any period of at least 90 minutes between the time of dosage and about 270 minutes following dosage that is at least 1.25-fold that observed when a control composition comprising an equivalent quantity of undispersed drug is dosed. Thus, the compositions of the present invention can be evaluated in either an in vitro or in vivo test, or both.

A typical test to evaluate enhanced drug concentration can be conducted by (1) dissolving a sufficient quantity of control composition, typically the GPI alone, in the in vitro test medium, typically MFD or PBS solution, to achieve equilibrium concentration of the GPI; (2) dissolving a sufficient quantity of test composition (e.g., the GPI and polymer) in an equivalent test medium, such that if all the GPI dissolved, the theoretical concentration of GPI would exceed the equilibrium concentration of the GPI by a factor of at least 2; and (3) determining whether the measured MDC of the test composition in the test medium is at least 1.25-fold that of the equilibrium concentration of the control composition. In conducting such a dissolution test, the amount of test composition or control composition used is an amount such that if all of the GPI dissolved the GPI concentration would be at least 2-fold to 100-fold that of the solubility of the GPI. The concentration of dissolved GPI is typically measured as a function of time by sampling the test medium and plotting GPI concentration in the test medium vs. time so that the MDC can be ascertained. To avoid GPI particulates which would give an erroneous determination, the test solution is either filtered or centrifuged. “Dissolved GPI” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred dispersions. It is recognized that this definition of “dissolved GPI” encompasses not only monomeric solvated GPI molecules but also a wide range of species such as polymer/GPI assemblies that have submicron dimensions such as GPI aggregates, aggregates of mixtures of polymer and GPI, micelles, polymeric micelles, colloidal particles or nanocrystals, polymer/GPI complexes, and other such GPI-containing species that are present in the filtrate or supernatant in the specified dissolution test.

Relative bioavailability of GPIs in the dispersions of the present invention can be tested in vivo in animals or humans using conventional methods for making such a determination. An in vivo test, such as a crossover study, may be used to determine whether a composition of GPI and polymer provides an enhanced relative bioavailability compared with a control composition comprised of a GPI but no polymer as described above. In an in vivo crossover study a “test composition” of GPI and polymer is dosed to half a group of test subjects and, after an appropriate washout period (e.g., one week) the same subjects are dosed with a “control composition” that comprises an equivalent quantity of GPI as the “test composition”. The other half of the group is dosed with the control composition first, followed by the test composition. The relative bioavailability is measured as the concentration in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC can be made by plotting the serum or plasma concentration of drug along the ordinate (y-axis) against time along the abscissa (x-axis). Generally, the values for AUC represent a number of values taken from all of the subjects in a patient test population averaged over the entire test population. A preferred embodiment of the invention is one in which the relative bioavailability of the test composition is at least 1.25 relative to a control composition comprised of a GPI but with no polymer as described above. (That is, the AUC provided by the test composition is at least 1.25-fold the AUC provided by the control composition.) An even more preferred embodiment of the invention is one in which the relative bioavailability of the test composition is at least 2.0 relative to a control composition of the GPI but with no polymer present, as described above. The determination of AUCs is a well-known procedure and is described, for example, in Welling, “Pharmacokinetics Processes and Mathematics,” ACS Monograph 185 (1986).

### GLYCOGEN PHOSPHORYLASE INHIBITORS

The invention is useful for GPIs which have sufficiently low aqueous solubility that it is desirable to increase their water solubility. Therefore, anytime one finds it desirable to raise the concentration of the GPI in a use environment, the invention will find utility. The GPI has “low-solubility,” meaning that the GPI may be either “substantially water-insoluble” (which means that the GPI has a minimum aqueous solubility at any physiologically relevant pH (e.g., pH 1-8) and about 22° C. of less than 0.01 mg/mL), or “sparingly water-soluble” (that is, has a water solubility up to about 1 mg/mL). (Unless otherwise specified, reference to aqueous solubility herein and in the claims is determined at about 22° C.) Compositions of the present invention find greater utility as the solubility of the GPI decreases, and thus are preferred for GPI solubilities less than 0.5 mg/mL, and even more preferred for GPI solubilities less than 0.1 mg/mL. In general, it may be said that the GPI has a dose-to-aqueous solubility ratio greater than about 10 mL, where the solubility (mg/mL) is the minimum value observed in any physiologically relevant aqueous solution (e.g., those with pH values from 1 to 8) including USP simulated gastric and intestinal buffers, and dose is in mg. Compositions of the present invention, as mentioned above, find greater utility as the solubility of the GPI decreases and the dose increases. Thus, the compositions are preferred as the dose-to-solubility ratio increases, and thus are preferred for dose-to-solubility ratios greater than 100 mL, and more preferred for dose-to-solubility ratios greater than 400 mL.

Preferably, the GPI binds to the GP enzyme at the indole pocket binding site. As used herein and in the claims, “bind” means a portion of the GPI binds to the GP enzyme in such a manner that a portion of the GPI is in van der Waals or hydrogen bonding contact with a portion or all portions of certain residues of the binding site. In a preferred embodiment, the GPI binds to the GP enzyme with a portion or all portions of the following residues of GP:

| parent | |
|---|---|
| secondary | |
| structure | residue number |
| | 13-23 |
| helix α1 | 24-37 |
| turn | 38-39, 43, 46-47 |
| helix α2 | 48-66, 69-70, 73-74, 76-78 |
| | 79-80 |
| strand β1 | 81-86 |
| | 87-88 |
| strand β2 | 89-92 |
| | 93 |
| helix α3 | 94-102 |
| | 103 |
| helix α4 | 104-115 |
| | 116-117 |
| helix α5 | 118-124 |
| | 125-128 |
| strand β3 | 129-131 |
| | 132-133 |
| helix α6 | 134-150 |
| | 151-152 |
| strand β4 | 153-160 |
| | 161 |
| strand β4b | 162-163 |
| | 164-166 |
| strand β5 | 167-171 |
| | 172-173 |
| strand β6 | 174-178 |
| | 179-190 |
| strand β7 | 191-192 |
| | 194, 197 |
| strand β8 | 198-209 |
| | 210-211 |
| strand β9 | 212-216 |
| strand β10 | 219-226, 228-232 |
| | 233-236 |
| strand β11 | 237-239, 241, 243-247 |
| | 248-260 |
| helix α7 | 261-276 |
| strand β11b | 277-281 |
| reverse turn | 282-289 |
| helix α8 | 290-304 |

More preferably, the GPI binds with one or more of the following residues of GP in one or both subunits:

| parent | |
|---|---|
| secondary | |
| structure | residue number |
| | 13-23 |
| helix α1 | 24-37 |
| turn | 38-39, 43, 46-47 |
| helix α2 | 48-66, 69-70, 73-74, 76-78 |
| | 79-80 |
| strand β2 | 91-92 |
| | 93 |
| helix α3 | 94-102 |
| | 103 |
| helix α4 | 104-115 |
| | 116-117 |
| helix α5 | 118-124 |
| | 125-128 |
| strand β3 | 129-130 |
| strand β4 | 159-160 |
| | 161 |
| strand β4b | 162-163 |
| | 164-166 |
| strand β5 | 167-168 |
| strand β6 | 178 |
| | 179-190 |
| strand β7 | 191-192 |
| | 194, 197 |
| strand β9 | 198-200 |
| strand β10 | 220-226 |
| | 228-232 |
| | 233-236 |
| strand β11 | 237-239, 241, 243-247 |
| | 248-260 |
| helix α7 | 261-276 |
| strand β11b | 277-280 |

Even more preferably, the GPI binds with one or more of the following residues of GP in one or both subunits:

residue number

33-39

49-66

94

98

102

125-126

160

162

182-192

197

224-226

228-231

238-239

241

245

247

Most preferably, the GPI binds with one or more of the following residues of GP in one or both subunits:

residue number

37-39

53

57

60

63-64

184-192

226

229

The indole pocket binding site is disclosed more fully in commonly assigned U.S. provisional patent application Ser. No. 95790 filed Aug. 7, 1998 and corresponding published European Patent Application No. EP0978279 A1, the relevant disclosure of which is herein incorporated by reference.

It is believed that certain compounds are capable of binding at the indole pocket binding site. Accordingly, preferred GPIs of the present invention are those that are capable of binding at this site. One such set of compounds has the structure of Formula I:

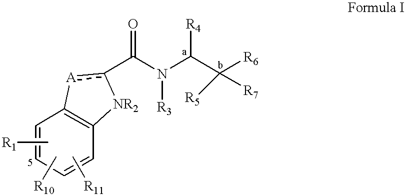

and the pharmaceutically acceptable salts and prodrugs thereof wherein the dotted line (- - -) is an optional bond, and the various substituents of Formula I are as follows;

A is —C(H)═, —C((C₁-C₄)alkyl)═ or —C(halo)═ when the dotted line (- - -) is a bond, or A is methylene or —CH((C₁-C₄)alkyl)— when the dotted line (- - -) is not a bond;

R₁, R₁₀ or R₁₁ are each independently H, halo, 4-, 6- or 7-nitro, cyano, (C₁-C₄)alkyl, (C₁-C₄)alkoxy, fluoromethyl, difluoromethyl or trifluoromethyl;

R₂ is H;

R₃ is H or (C₁-C₅)alkyl;

R₄ is methyl, ethyl, n-propyl, hydroxy(C₁-C₃)alkyl, (C₁-C₃)alkoxy(C₁-C₃)alkyl, phenyl(C₁-C₄)alkyl, phenylhydroxy(C₁-C₄)alkyl, phenyl(C₁-C₄)alkoxy(C₁-C₄)alkyl, thien-2- or -3-yl(C₁-C₄)alkyl or fur-2- or -3-yl(C₁-C₄)alkyl wherein said R₄ rings are mono-, di- or tri-substituted independently on carbon with H, halo, (C₁-C₄)alkyl, (C₁-C₄)alkoxy, trifuloromethyl, hydroxy, amino or cyano; or

R₄ is pyrid-2-, -3- or -4-yl(C₁-C₄)alkyl, thiazol-2-, -4- or -5-yl(C₁-C₄)alkyl, imidazol-1-, -2-, -4- or -5-yl(C₁-C₄)alkyl, pyrrol-2- or -3-yl(C₁-C₄)alkyl, oxazol-2-, -4- or -5-yl(C₁-C₄)alkyl, pyrazol-3-, -4- or -5-yl(C₁-C₄)alkyl, isoxazol-3-, -4-, -5-yl(C₁-C₄)alkyl, isothiazol-3-, -4-, -5-yl(C₁-C₄)alkyl, pyridazin-3- or -4-yl-(C₁-C₄)alkyl, pyrimidin-2-, -4-, -5- or -6-yl(C₁-C₄)alkyl, pyrazin-2- or -3-yl(C₁-C₄)alkyl or 1,3,5-triazin-2-yl(C₁-C₄)alkyl, wherein said preceding R₄ heterocycles are optionally mono- or di-substituted independently with halo, trifluoromethyl, (C₁-C₄)alkyl, (C₁-C₄)alkoxy, amino or hydroxy and said mono- or di-substituents are bonded to carbon;

R₅ is H, hydroxy, fluoro, (C₁-C₅)alkyl, (C₁-C₅)alkoxy, (C₁-C₆)alkanoyl, amino (C₁-C₄) alkoxy, mono-N- or di-N,N-(C₁-C₄)alkylamino(C₁-C₄)alkoxy, carboxy(C₁-C₄)alkoxy, (C₁-C₅)alkoxy-carbonyl(C₁-C₄) alkoxy, benzyloxycarbonyl (C₁-C₄) alkoxy, or carbonyloxy wherein said carbonyloxy is carbon-carbon linked with phenyl, thiazolyl, imidazolyl, 1H-indolyl, furyl, pyrrolyl, oxazolyl, pyrazolyl, isoxazolyl, isothiazolyl, pvridazinyl, pyrimidinyl, pyrazinyl or 1,3,5-triazinyl and wherein said preceding R₅ rings are optionally mono-substituted with halo, (C₁-C₄)alkyl, (C₁-C₄)alkoxy, hydroxy, amino or trifluoromethyl and said mono-substituents are bonded to carbon;

R₇ is H, fluoro or (C₁-C₅)alkyl; or

R₅ and R₇ can be taken together to be oxo;

R₆ is carboxy, (C₁-C₈)alkoxycarbonyl, C(O)NR₈R₉ or C(O)R₁₂ wherein

R₈ is (C₁-C₃)alkyl, hydroxy or (C₁-C₃)alkoxy; and

R₉ is H, (C₁-C₈)alkyl, hydroxy, (C₁-C₈)alkoxy, methylene-perfluorinated(C₁-C₈)alkyl, phenyl, pyridyl, thienyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, piperidinyl, morpholinyl, pyridazinyl, pyrimidinyl, pyrazinyl, piperazinyl or 1,3,5-triazinyl wherein said preceding R₉ rings are carbon-nitrogen linked; or

R₉ is mono-, di- or tri-substituted (C₁-C₅)alkyl, wherein said substituents are independently H, hydroxy, amino, mono-N- or di-N,N-(C₁-C₅)alkylamino; or

R₉ is mono- or di-substituted (C₁-C₅)alkyl, wherein said substituents are independently phenyl, pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, pyridinyl, piperidinyl, morpholinyl, pyridazinyl, pyrimidinyl, pyrazinyl, piperazinyl or 1,3,5-triazinyl

wherein the nonaromatic nitrogen-containing R₉ rings are optionally mono-substituted on nitrogen with (C₁-C₆)alkyl, benzyl, benzoyl or (C₁-C₆)alkoxycarbonyl and wherein the R₉ rings are optionally mono-substituted on carbon with halo, (C₁-C₄)alkyl, (C₁-C₄)alkoxy, hydroxy, amino, or mono-N- and di-N,N (C₁-C₅)alkylamino provided that no quaternized nitrogen is included and there are no nitrogen-oxygen, nitrogen-nitrogen or nitrogen-halo bonds;

R₁₂ is piperazin-1-yl, 4-(C₁-C₄)alkylpiperazin-1-yl, 4-formylpiperazin-1-yl, morpholino, thiomorpholino, 1-oxothiomorpholino, 1,1-dioxo-thiomorpholino, thiazolidin-3-yl, 1-oxo-thiazolidin-3-yl, 1,1-dioxo-thiazolidin-3-yl, 2-(C₁-C₆)alkoxycarbonylpyrrolidin-1-yl, oxazolidin-3-yl or 2(R)-hydroxymethylpyrrolidin-1-yl; or

R₁₂ is 3- and/or 4-mono- or di-substituted oxazetidin-2-yl, 2-, 4-, and/or 5-mono- or di-substituted oxazolidin-3-yl, 2-, 4-, and/or 5-mono- or di-substituted thiazolidin-3-yl, 2-, 4- and/or 5-mono- or di-substituted 1-oxothiazolidin-3-yl, 2-, 4-, and/or 5-mono- or di-substituted 1,1-dioxothiazolidin-3-yl, 3- and/or 4-, mono- or di-substituted pyrrolidin-1-yl, 3-, 4- and/or 5-, mono-, di- or tri-substituted piperidin-1-yl, 3-, 4-, and/or 5-mono-, di-, or tri-substituted piperazin-1-yl, 3-substituted azetidin-1-yl, 4- and/or 5-, mono- or di-substituted 1,2-oxazinan-2-yl, 3-and/or 4-mono- or di-substituted pyrazolidin-1-yl, 4- and/or 5-, mono- or di-substituted isothiazolidin-2-yl, 4- and/or 5-, mono- and/or di-substituted isothiazolidin-2-yl wherein said R₁₂ substituents are independently H, halo, (C₁-C₅)alkyl, hydroxy, amino, mono-N- or di-N,N-(C₁-C₅)alkylamino, formyl, oxo, hydroxyimino, (C₁-C₅)alkoxy, carboxy, carbamoyl, mono-N-or di-N,N-(C₁-C₄)alkylcarbamoyl, (C₁-C₄)alkoxyimino, (C₁-C₄)alkoxymethoxy, (C₁-C₆)alkoxycarbonyl, carboxy(C₁-C₅)alkyl or hydroxy (C₁-C₅) alkyl;

with the proviso that if R₄ is H, methyl, ethyl or n-propyl, R₅ is OH;

with the proviso that if R₅ and R₇ are H, then R₄ is not H, methyl, ethyl, n—propyl, hydroxy(C₁-C₃)alkyl or (C₁-C₃)alkoxy(C₁-C₃)alkyl and R₆ is C(O)NR₈R₉, C(O)R₁₂ or (C₁-C₄) alkoxycarbonyl.

Compounds of Formula I are disclosed in published Patent Cooperation Treaty Application number WO 96/39385, the complete disclosure of which is hereby incorporated by reference.

In yet another preferred aspect of the invention, the GPI has the structure of Formula II, which is another class of compounds thought capable of binding to the indole pocket binding site:

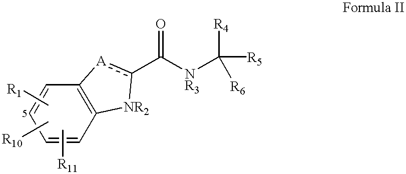

and the pharmaceutically acceptable salts and prodrugs thereof wherein the dotted line (- - -) is an optional bond and the substituents of Formula II are as follows:

A is —C(H)═, —C((C₁-C₄)alkyl)═, —C(halo)═ or —N═, when the dotted line (- - -) is a bond, or A is methylene or —CH((C₁-C₄)alkyl)—, when the dotted line (- - -) is not a bond;

R₁, R₁₀ or R₁₁ are each independently H, halo, cyano, 4-, 6- or 7-nitro, (C₁-C₄)alkyl, (C₁-C₄)alkoxy, fluoromethyl, difluoromethyl or trifluoromethyl;

R₂ is H;

R₃ is H or (C₁-C₅)alkyl;

R₄ is H, methyl, ethyl, n-propyl, hydroxy(C₁-C₃)alkyl, (C₁-C₃)alkoxy(C₁-C₃)alkyl, phenyl(C₁-C₄)alkyl, phenylhydroxy(C₁-C₄)alkyl, (phenyl)((C₁-C₄)-alkoxy)(C₁-C₄)alkyl, thien-2- or -3-yl(C₁-C₄)alkyl or fur-2- or -3-yl(C₁-C₄)alkyl wherein said R₄ rings are mono-, di- or tri-substituted independently on carbon with H, halo, (C₁-C₄)alkyl, (C₁-C₄)alkoxy, trifuloromethyl, hydroxy, amino, cyano or 4,5-dihydro-1H-imidazol-2-yl; or

R₄ is pyrid-2-, -3- or -4-yl(C₁-C₄)alkyl, thiazol-2-, -4- or -5-yl(C₁-C₄)alkyl, imidazol-2-, -4-, or -5-yl(C₁-C₄)alkyl, pyrrol-2- or -3-yl(C₁-C₄)alkyl, oxazol-2-, -4- or -5-yl(C₁-C₄)alkyl, pyrazol-3-, -4- or -5-yl(C₁-C₄)alkyl, isoxazol-3-, -4- or -5-yl(C₁-C₄)alkyl, isothiazol-3-, -4- or -5-yl(C₁-C₄)alkyl, pyridazin-3- or -4-yl(C₁-C₄)alkyl, pyrimidin-2-, -4-, -5- or -6-yl(C₁-C₄)alkyl, pyrazin-2- or -3-yl(C₁-C₄)alkyl, 1,3,5-triazin-2-yl(C₁-C₄)alkyl or indol-2-(C₁-C₄)alkyl, wherein said preceding R₄ heterocycles are optionally mono- or di-substituted independently with halo, trifluoromethyl, (C₁-C₄)alkyl, (C₁-C₄)alkoxy, amino, hydroxy or cyano and said substituents are bonded to carbon; or

R₄ is R₁₅-carbonyloxymethyl, wherein said R₁₅ is phenyl, thiazolyl, imidazolyl, 1H-indolyl, furyl, pyrrolyl, oxazolyl, pyrazolyl, isoxazolyl, isothiazolyl, pyridyl, pyridazinyl, pyrimidinyl, pyrazinyl or 1,3,5-triazinyl and wherein said preceding R₁₅ rings are optionally mono- or di-substituted independently with halo, amino, hydroxy, (C₁-C₄)alkyl, (C₁-C₄)alkoxy or trifluoromethyl and said mono- or di-substituents are bonded to carbon;

R₅ is H, methyl, ethyl, n-propyl, hydroxymethyl or hydroxyethyl;

R₆ is carboxy, (C₁-C₈)alkoxycarbonyl, benzyloxycarbonyl, C(O)NR₈R₉ or C(O)R₁₂

wherein R₈ is H, (C₁-C₆)alkyl, cyclo (C₃-C₆)alkyl, cyclo(C₃-C₆)alkyl(C₁-C₅)alkyl, hydroxy or (C₁-C₈) alkoxy; and

R₉ is H, cyclo(C₃-C₆)alkyl, cyclo(C₃-C₈)alkyl (C₁-C₅)alkyl, cyclo (C₄-C₇) alkenyl, cyclo(C₃-C₇)alkyl(C₁-C₅)alkoxy, cyclo(C₃-C₇)alkyloxy, hydroxy, methylene-perfluorinated (C₁-C₈)alkyl, phenyl, or a heterocycle wherein said heterocycle is pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, pyridinyl, piperidinyl, morpholinyl, pyridazinyl, pyrimidinyl, pyrazinyl, piperazinyl, 1,3,5-triazinyl, benzothiazolyl, benzoxazolyl, benzimidazolyl, thiochromanyl or tetrahydrobenzothiazolyl wherein said heterocycle rings are carbon-nitrogen linked; or

R₉ is (C₁-C₆)alkyl or (C₁-C₈)alkoxy wherein said (C₁-C₆)alkyl or (C₁-C₈)alkoxy is optionally monosubstituted with cyclo(C₄-C₇)alken-1-yl, phenyl, thienyl, pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, piperidinyl, morpholinyl, thiomorpholinyl, 1-oxothiomorpholinyl, 1,1-dioxothiomorpholinyl, pyridazinyl, pyrimidinyl, pyrazinyl, piperazinyl, 1,3,5-triazinyl or indolyl and wherein said (C₁-C₆)alkyl or (C₁-C₈)alkoxy are optionally additionally independently mono- or di-substituted with halo, hydroxy, (C₁-C₅)alkoxy, amino, mono-N- or di-N,N-(C₁-C₅)alkylamino, cyano, carboxy, or (C₁-C₄)alkoxycarbonyl; and

wherein the R₉ rings are optionally mono- or di-substituted independently on carbon with halo, (C₁-C₄)alkyl, (C₁-C₄)alkoxy, hydroxy, hydroxy(C₁-C₄)alkyl, amino(C₁-C₄)alkyl, mono-N- or di-N,N-(C₁-C₄)alkylamino (C₁-C₄)alkyl, (C₁-C₄)alkoxy(C₁-C₄)alkyl, amino, mono-N- or di-N,N-(C₁-C₄)alkylamino, cyano, carboxy, (C₁-C₅)alkoxycarbonyl, carbamoyl, formyl or trifluoromethyl and said R₉ rings may optionally be additionally mono- or di-substituted independently with (C₁-C₅)alkyl or halo;

with the proviso that no quaternized nitrogen on any R₉ heterocycle is included;

R₁₂ is morpholino, thiomorpholino, 1-oxothiomorpholino, 1,1-dioxothiomorpholino, thiazolidin-3-yl, 1-oxothiazolidin-3-yl, 1,1-dioxothiazolidin-3-yl, pyrrolidin-1-yl, piperidin-1-yl, piperazin-1-yl, piperazin-4-yl, azetidin-1-yl, 1,2-oxazinan-2-yl, pyrazolidin-1-yl, isoxazolidin-2-yl, isothiazolidin-2-yl, 1,2-oxazetidin-2-yl, oxazolidin-3-yl, 3,4-dihydroisoquinolin-2-yl, 1,3-dihydroisoindol-2-yl, 3,4-dihydro-2H-quinol-1-yl, 2,3-dihydro-benzo[1,4]oxazin-4-yl, 2,3-dihydro-benzo[1,4]-thiazine-4-yl, 3,4-dihydro-2H-quinoxalin-1-yl, 3,4-dihydro-benzo[c][1,2]oxazin-1-yl, 1,4-dihydro-benzo[d][1,2]oxazin-3-yl, 3,4-dihydro-benzo[e][1,2]-oxazin-2-yl, 3H-benzo[d]isoxazol-2-yl, 3H-benzo[c]isoxazol-1-yl or azepan-1-yl,

wherein said R₁₂ rings are optionally mono-, di- or tri-substituted independently with halo, (C₁-C₅)alkyl, (C₁-C₅)alkoxy, hydroxy, amino, mono-N- or di-N,N-(C₁-C₅)alkylamino, formyl, carboxy, carbamoyl, mono-N- or di-N,N-(C₁-C₅)alkylcarbamoyl, (C₁-C₆)alkoxy (C₁-C₃) alkoxy, (C₁-C₅)alkoxycarbonyl, benzyloxycarbonyl, (C₁-C₅) alkoxycarbonyl (C₁-C₅) alkyl, (C₁-C₄)alkoxycarbonylamino, carboxy(C₁-C₅)alkyl, carbamoyl(C₁-C₅)alkyl, mono-N- or di-N,N-(C₁-C₅)alkylcarbamoyl(C₁-C₅)alkyl, hydroxy(C₁-C₅)alkyl, (C₁-C₄)alkoxy(C₁-C₄)alkyl, amino(C₁-C₄)alkyl, mono-N- or di-N,N-(C₁-C₄)alkylamino(C₁-C₄)alkyl, oxo, hydroxyimino or (C₁-C₆)alkoxyimino and wherein no more than two substituents are selected from oxo, hydroxyimino or (C₁-C₆)alkoxyimino and oxo, hydroxyimino or (C₁-C₆)alkoxyimino are on nonaromatic carbon; and

wherein said R₁₂ rings are optionally additionally mono- or di-substituted independently with (C₁-C₅)alkyl or halo;

with the proviso that when R₆ is (C₁-C₅)alkoxycarbonyl or benzyloxycarbonyl then R₁ is 5-halo, 5-(C₁-C₄)alkyl or 5-cyano and R₄ is (phenyl) (hydroxy) (C₁-C₄)alkyl,

(phenyl) ((C₁-C₄) alkoxy) (C₁-C₄)alkyl, hydroxymethyl or Ar(C₁-C₂)alkyl, wherein Ar is thien-2- or -3-yl, fur-2- or -3-yl or phenyl wherein said Ar is optionally mono- or di-substituted independently with halo; with the provisos that when R₄ is benzyl and R₅ is methyl, R₁₂ is not 4-hydroxy-piperidin-1-yl or when R₄ is benzyl and R₅ is methyl R₆ is not C(O)N(CH₃)₂;

with the proviso that when R₁ and R₁₀ and R₁₁ are H, R₄ is not imidazol-4-ylmethyl, 2-phenylethyl or 2-hydroxy-2-phenylethyl;

with the proviso that when R₈ and R₉ are n-pentyl, R₁ is 5-chloro, 5-bromo, 5-cyano, 5(C₁-C₅)alkyl, 5(C₁-C₅)alkoxy or trifluoromethyl;

with the proviso that when R₁₂ is 3,4-dihydroisoquinol-2-yl, said 3,4-dihydroisoquinol-2-yl is not substituted with carboxy((C₁-C₄)alkyl;

with the proviso that when R₈ is H and R₉ is (C₁-C₆)alkyl, R₉ is not substituted with carboxy or (C₁-C₄)alkoxycarbonyl on the carbon which is attached to the nitrogen atom N of NHR₉; and

with the proviso that when R₆ is carboxy and R₁, R₁₀, R₁₁ and R₅ are all H, then R₄ is not benzyl, H, (phenyl)(hydroxy)methyl, methyl, ethyl or n-propyl.

Compounds of Formula II are disclosed in published Patent Cooperation Treaty Publication number WO 96/39384, the complete disclosure of which is hereby incorporated by reference.

In yet another preferred aspect of the invention, the GPI has the structure of Formula III, which is another class of compounds believed to be capable of binding to the indole pocket binding site:

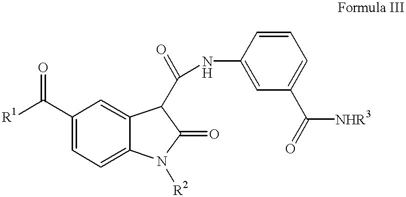

a prodrug thereof or a pharmaceutically acceptable salt of said compound or said prodrug wherein Formula III has the following substituents:

R¹ is (C₁-C₄)alkyl, (C₃-C₇) cycloalkyl, phenyl or phenyl substituted with up to three (C₁-C₄)alkyl, (C₁-C₄) alkoxy or halogen;

R² is (C₁-C₄)alkyl; and

R₃ is (C₃-C₇)cycloalkyl; phenyl; phenyl substituted at the para position with (C₁-C₄)alkyl, halo, hydroxy(C₁-C₄)alkyl or trifluoromethyl; phenyl substituted at the meta position with fluoro; or phenyl substituted at the ortho position with fluoro.

Compounds of formula III are disclosed more fully in commonly assigned U.S. Pat. No. 5,998,463, the relevant disclosure of which is incorporated by reference.

In yet another preferred aspect of the invention, the GPI has the structure of Formula IV, which is another class of compounds believed to be capable of binding to the indole pocket binding site:

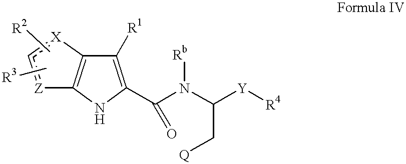

a stereoisomer, pharmaceutically acceptable salt or prodrug thereof, or a pharmaceutically acceptable salt of the prodrug, wherein Formula IV has the following substituents:

Q is aryl, substitued aryl, heteroaryl, or substitued heteroaryl;

each Z and X are independently (C, CH or CH₂), N, O or S;

X¹ is NR, O or S;

each - - - - is independently a bond or is absent, provided that both - - - - are not simlutaneously bonds;

R² is hydrogen, halogen, —OC₁-C₈alkyl, —SC₁₋-C₈alkyl, —C₁-C₈alkyl, —CF₃, —NH₂, —NHC₁-C₈alkyl, —N(C₁-C₈alkyl)₂, —NO₂, —CN,

—CO₂H, —CO₂C₁-C₈alkyl, —C₂-C₈alkenyl, or —C₂-C₈alkynyl;

each R^{a} and R^{b} is independently hydrogen or —C₁-C₈alkyl;

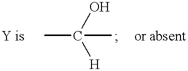

R² and R³ are independently hydrogen, halogen, —C₁-C₈alkyl, —CN, —C≡C—Si(CH₃)₃, —OC₁-C₈alkyl, —SC₁-C₈alkyl, —CF₃, —NH₂, —NHC₁-C₈alkyl, —N(C₁-C₈alkyl)₂, —NO₂, —CO₂H, —CO₂C₁-C₈alkyl, —C₂-C₈alkenyl, or —C₂-C₈alkynyl, or R² and R³ together with the atoms on the ring to which they are attached form a five or six membered ring containing from 0 to 3 heteroatoms and from 0 to 2 double bonds;

R⁴ is —C(═O)—A;

A is —NR^{d}R^{d}, —NR^{a}CH₂CH₂OR^{a},

[chemical structure]

each R^{d} is independently hydrogen, C₁-C₈alkyl, C₁-C₈alkoxy, aryl, substituted aryl, heteroaryl, or substituted heteroaryl;

each R^{c} is independently hydrogen, —C(═O)OR^{a}, —OR^{a}, —SR^{a}, or NR^{a}R^{a}; and

each n is independently 1-3.

Compounds of Formula IV are disclosed in commonly assigned U.S. Provisional Patent Application Ser. No. 60/157,148, filed Sep. 30, 1999, the relevant disclosure of which is incorporated by reference.

In an especially preferred embodiment, the GPI is selected from one of the following compounds of Formula I:

5-chloro-1H-indole-2-carboxylic acid [(1S)-((R)-hydroxy-dimethylcarbamoylmethyl)-2-phenyl-ethyl]-amide;

5-chloro-1H-indole-2-carboxylic acid [(1S)-((R)-hydroxy-methoxy-methylcarbamoylmethyl)-2-phenyl-ethyl]-amide;

5-chloro-1H-indole-2-carboxylic acid [(1S)-benzyl-(2R)-hydroxy-3-((3S)-hydroxy-pyrrolidin-1-yl)-3-oxo-propyl]-amide;

5-chloro-1H-indole-2-carboxylic acid [(1S)-benzyl-(2R)-hydroxy-3-((3R,4S)-dihydroxy-pyrrolidin-1-yl)-3-oxo-propyl]-amide;

5-chloro-1H-indole-2-carboxylic acid [(1S)-benzyl-(2R)-hydroxy-3-((3R,4R)-dihydroxy-pyrrolidin-1-yl)-3-oxo-propyl]-amide; and

5-chloro-1H-indole-2-carboxylic acid [(1S)-benzyl-(2R)-hydroxy-3-morpholin-4-yl-3-oxo-propyl]-amide.

In another especially preferred embodiment, the GPI is selected from one of the following compounds of Formula II:

5-chloro-1H-indole-2-carboxylic acid [2-((3R,4S)-3,4-dihydroxy-pyrrolidin-1-yl)-2-oxo-ethyl]-amide;

5-chloro-1H-indole-2-carboxylic acid [(1S)-benzyl-2-((3S,4S)-3,4-dihydroxy-pyrrolidin-1-yl)-2-oxo-ethyl]-amide;

5-chloro-1H-indole-2-carboxylic acid [(1S)-benzyl-2-((3R,4S)-3,4-dihydroxy-pyrrolidin-1-yl)-2-oxo-ethyl]-amide;

5-chloro-1H-indole-2-carboxylic acid [(1S)-(4-fluoro-benzyl)-2-(4-hydroxy-piperidin-1-yl)-2-oxo-ethyl]-amide;

5-chloro-1H-indole-2-carboxylic acid [(1S)-benzyl-2-(3-hydroxy-azetidin-1-yl)-2-oxo-ethyl]-amide;

5-chloro-1H-indole-2-carboxylic acid [2-(1,1-dioxo-thiazolidin-3-yl)-2-oxo-ethyl]-amide; and

5-chloro-1H-indole-2-carboxylic acid [2-(1-oxo-thiazolidin-3-yl)-2-oxo-ethyl]-amide.

In another especially preferred embodiment, the GPI is selected from one of the following compounds of Formula III:

5-acetyl-1-ethyl-2,3-dihydro-2-oxo-N-[3-[(phenylamino)carbonyl]phenyl]-1H-Indole-3-carboxamide;

5-acetyl-N-[3-[(cyclohexylamino)carbonyl]phenyl-1-ethyl-2,3-dihydro-2-oxo-1H-Indole-3-carboxamide; and

5-acetyl-N-[3-[[(4-bromophenyl)amino]carbonyl]phenyl]-2,3-dihydro-1-methyl-2-oxo-1H-Indole-3-carboxamide.

In another especially preferred embodiment, the GPI is selected from one of the following compounds of Formula IV:

2-Chloro-6H-thieno[2,3-b]pyrrole-5-carboxylic acid [(1S)-benzyl-2-((3R,4S)-dihydroxy-pyrrolidin-1-yl)-2-oxo-ethyl]-amide; and

2-chloro-6H-thieno[2,3-b]pyrrole-5-carboxylic acid [(1S)-benzyl-(2R)-hydroxy-3-((3R,4S)-dihydroxy-pyrrolidin-1-yl)-3-oxo-propyl]-amide.

### CONCENTRATION-ENHANCING POLYMERS

Concentration-enhancing polymers suitable for use in the compositions of the present invention should be inert, in the sense that they do not chemically react with the GPI in an adverse manner, are pharmaceutically acceptable, and have at least some solubility in aqueous solution at physiologically relevant pHs (e.g. 1-8). The polymer can be neutral or ionizable, and should have an aqueous-solubility of at least 0.1 mg/mL over at least a portion of the pH range of 1-8. The polymer is a “concentration-enhancing polymer,” meaning that it meets at least one, and more preferably both, of the following conditions. The first condition is that the concentration-enhancing polymer increases the MDC of the GPI in the environment of use relative to a control composition consisting of an equivalent amount of the GPI but no polymer. That is, once the composition is introduced into an environment of use, the polymer increases the aqueous concentration of GPI relative to the control composition. Preferably, the polymer increases the MDC of the GPI in aqueous solution by at least 1.25-fold relative to a control composition, and more preferably by at least 2-fold and most preferably by at least 3-fold. The second condition is that the concentration-enhancing polymer increases the AUC of the GPI in the environment of use relative to a control composition consisting of GPI but no polymer as described above. That is, in the environment of use, the composition comprising the GPI and the concentration-enhancing polymer provides an area under the concentration versus time curve (AUC) for any period of 90 minutes between the time of introduction into the use environment and about 270 minutes following introduction to the use environment that is at least 1.25-fold that of a control composition comprising an equivalent quantity of GPI but no polymer.

Concentration-enhancing polymers suitable for use with the present invention may be cellulosic or non-cellulosic. The polymers may be neutral or ionizable in aqueous solution of these, ionizable and cellulosic polymers are preferred, with ionizable cellulosic polymers being more preferred.

A preferred class of polymers comprises polymers that are “amphiphilic” in nature, meaning that the polymer has hydrophobic and hydrophilic portions. Hydrophobic groups may comprise groups such as aliphatic or aromatic hydrocarbon groups. Hydrophilic groups may comprise either ionizable or non-ionizable groups that are capable of hydrogen bonding such as hydroxyls, carboxylic acids, esters, amines or amides.

Amphiphilic and/or ionizable polymers are preferred because it is believed that such polymers may tend to have relatively strong interactions with the GPI and may promote the formation of the various types of polymer/drug assemblies in the use environment as described previously. In addition, the repulsion of the like charges of the ionized groups of such polymers may serve to limit the size of the polymer/drug assemblies to the nanometer or submicron scale. For example, while not wishing to be bound by a particular theory, such polymer/drug assemblies may comprise hydrophobic GPI clusters surrounded by the polymer with the polymer's hydrophobic regions turned inward towards the GPI and the hydrophilic regions of the polymer turned outward toward the aqueous environment. Alternatively, depending on the specific chemical nature of the GPI, the ionized functional groups of the polymer may associate, for example, via ion pairing or hydrogen bonds, with ionic or polar groups of the GPI. In the case of ionizable polymers, the hydrophilic regions of the polymer would include the ionized functional groups. Such polymer/drug assemblies in solution may well resemble charged polymeric micellar-like structures. In any case, regardless of the mechanism of action, the inventors have observed that such amphiphilic polymers, particularly ionizable cellulosic polymers, have been shown to improve the MDC and/or AUC of GPI in aqueous solution relative to control compositions free from such polymers.

Surprisingly, such amphiphilic polymers can greatly enhance the maximum concentration of GPI obtained when an amorphous form of the GPI is dosed to a use environment. In addition, such amphiphilic polymers interact with the GPI to prevent the precipitation or crystallization of the GPI from solution despite its concentration being substantially above its equilibrium concentration. In particular, when the preferred compositions are solid amorphous dispersions of the GPI and the concentration-enhancing polymer, the compositions provide a greatly enhanced drug concentration, particularly when the dispersions are substantially homogeneous. The maximum drug concentration may be 2-fold and often up to 10-fold the equilibrium concentration of the crystalline GPI. Such enhanced GPI concentrations in turn lead to substantially enhanced relative bioavailability for the GPI.

One class of polymers suitable for use with the present invention comprises neutral non-cellulosic polymers. Exemplary polymers include: vinyl polymers and copolymers having substituents of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; and polyethylene polyvinyl alcohol copolymers.

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Rohm Tech Inc., of Malden, Mass.; amine-functionalized polyacrylates and polymethacrylates; proteins; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers. Exemplary commercial grades of such copolymers include the EUDRAGITS, which are copolymers of methacrylates and acrylates.

A preferred class of polymers comprises ionizable and neutral cellulosic polymers with at least one ester- and/or ether-linked substituent in which the polymer has a degree of substitution of at least 0.1 for each substituent. It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analagously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.1 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer.

Amphiphilic cellulosics may be prepared by substituting the cellulosic at any or all of the 3 hydroxyl substituents present on each saccharide repeat unit with at least one relatively hydrophobic substituent. Hydrophobic substituents may be essentially any substituent that, if substituted to a high enough level or degree of substitution, can render the cellulosic polymer essentially aqueous insoluble. Hydrophilic regions of the polymer can be either those portions that are relatively unsubstituted, since the unsubstituted hydroxyls are themselves relatively hydrophilic, or those regions that are substituted with hydrophilic substituents. Examples of hydrophobic substitutents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic groups include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable groups include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

A preferred set of neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A preferred class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic group may itself be ionizable, such as phthalate or trimellitate substituents.

Exemplary ionizable cellulosic polymers that are at least partially ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary cellulosic polymers that meet the definition of amphiphilic, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A particularly desirable subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxylpropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another particularly desirable subset of cellulosic ionizable polymers are those that possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, and hydroxyethyl cellulose acetate succinate.

Especially preferred polymers are hydroxypropyl methyl cellulose acetate succinate (HPMCAS), hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate and cellulose acetate isophthalate. The most preferred polymers are hydroxypropyl methyl cellulose acetate succinate, hydroxypropyl methyl cellulose phthalate, cellulose acetate phthalate, and cellulose acetate trimellitate.

While specific polymers have been discussed as being suitable for use in the mixtures of the present invention, blends of such polymers may also be suitable. Thus the term “polymer” is intended to include blends of polymers in addition to a single species of polymer.

To obtain the best performance, particularly upon storage for long times prior to use, it is preferred that the GPI remain, to the extent possible, in the amorphous state. The inventors have found that this is best achieved when the glass-transition temperature, T_{g} of the amorphous GPI material is substantially above the storage temperature of the composition. In particular, it is preferable that the T_{g} of the amorphous state of the GPI be at least 40° C. and preferably greater than 60° C. For those aspects of the invention in which the composition is a solid, substantially amorphous dispersion of GPI in the concentration-enhancing polymer and in which the GPI itself has a relatively low T_{g} (about 70° C. or less) it is preferred that the concentration-enhancing polymer have a T_{g} of at least 40° C., preferably at least 70° C. and more preferably greater than 100° C. Exemplary high T_{g} polymers include HPMCAS, HPMCP, CAP, CAT and other cellulosics that have alkylate or aromatic substituents or both alkylate and aromatic substituents.

In addition, the preferred polymers listed above, that is amphiphilic cellulosic polymers, tend to have greater concentration-enhancing properties relative to the other polymers of the present invention. For any particular GPI, the amphiphilic cellulosic with the best concentration-enhancing properties may vary. However, the inventors have found that generally those that have ionizable substituents tend to perform best. In vitro tests of compositions with such polymers tend to have higher MDC and AUC values than compositions with other polymers of the invention. Often such compositions have MDC and AUC values that are more than 4-fold and in some cases more than 8-fold that of a control composition.

### PREPARATION OF COMPOSITIONS

Compositions may comprise a physical mixture of GPI and concentration-enhancing polymer or a dispersion of GPI and polymer. Preferably, the compositions are formed such that at least a major portion (at least 60%) of the GPI is in the amorphous state. In cases where the composition is a physical mixture of amorphous GPI and polymer the amorphous GPI may be made by any known process. Generally the amorphous form of the GPI is made by (1) melting the drug followed by rapid cooling (e.g., melt-congeal process); (2) dissolution of the drug in a solvent followed by precipitation or evaporation (e.g., spray drying, spray coating); or (3) mechanical processing of the drug (e.g., extrusion, ball milling). Various combinations of heat (as in melt processes), solvent and mechanical force may be used to generate the amorphous GPI.

Dispersions of the GPI and concentration-enhancing polymer may be made according to any known process which results in at least a major portion (at least 60%) of the GPI being in the amorphous state. Exemplary mechanical processes include milling and extrusion; melt processes include high temperature fusion, solvent modified fusion and melt-congeal processes; and solvent processes include non-solvent precipitation, spray coating and spray-drying. Although the dispersions of the present invention may be made by any of these processes, the dispersions generally have their maximum bioavailability and stability when the GPI is dispersed in the polymer such that it is substantially amorphous and substantially homogeneously distributed throughout the polymer. Although in some cases such substantially amorphous and substantially homogeneous dispersions may be made by any of these methods, it has been found that such dispersions are preferably formed by “solvent processing,” which consists of dissolution of the GPI and one or more polymers in a common solvent. “Common” here means that the solvent, which can be a mixture of compounds, will simultaneously dissolve the drug and the polymer(s). After both the GPI and the polymer have been dissolved, the solvent is rapidly removed by evaporation or by mixing with a non-solvent. Exemplary processes are spray-drying, spray-coating (pan-coating, fluidized bed coating, etc.), and precipitation by rapid mixing of the polymer and drug solution with CO₂, water, or some other non-solvent. Preferably, removal of the solvent results in a solid dispersion which is substantially homogeneous. As described previously, in such substantially homogeneous dispersions, the GPI is dispersed as homogeneously as possible throughout the polymer and can be thought of as a solid solution of GPI dispersed in the polymer(s). When the resulting dispersion constitutes a solid solution of GPI in polymer, the dispersion may be thermodynamically stable, meaning that the concentration of GPI in the polymer is at or below its equilibrium value, or it may be considered a supersaturated solid solution where the GPI concentration in the dispersion polymer(s) is above its equilibrium value.

The solvent may be removed through the process of spray-drying. The term spray-drying is used conventionally and broadly refers to processes involving breaking up liquid mixtures into small droplets (atomization) and rapidly removing solvent from the mixture in a container (spray-drying apparatus) where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. This is accomplished by either (1) maintaining the pressure in the spray-drying apparatus at a partial vacuum (e.g., 0.01 to 0.50 atm); (2) mixing the liquid droplets with a warm drying gas; or (3) both. In addition, at least a portion of the heat required for evaporation of solvent may be provided by heating the spray solution.

Solvents suitable for spray-drying can be any organic compound in which the GPI and polymer are mutually soluble. Preferably, the solvent is also volatile with a boiling point of 150° C. or less. In addition, the solvent should have relatively low toxicity and be removed from the dispersion to a level that is acceptable according to The International Committee on Harmonization (ICH) guidelines. Removal of solvent to this level may require a processing step such as tray-drying subsequent to the spray-drying or spray-coating process. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethyl acetamide or dimethylsulfoxide can also be used. Mixtures of solvents, such as 50% methanol and 50% acetone, can also be used, as can mixtures with water as long as the polymer and GPI are sufficiently soluble to make the spray-drying process practicable. Generally, non-aqueous solvents are preferred meaning that the solvent comprises less than about 40 wt % water. However, for certain GPIs, it has been found that addition of a small amount of water, typically about 5 wt % to about 35 wt %, to a solvent such as acetone may actually increase the solubility of the GPI in the solvent, relative to that in the absence of water. In such cases, or to enhance the polymer solubility, addition of water may even be preferred.

Generally, the temperature and flow rate of the drying gas is chosen so that the polymer/drug-solution droplets are dry enough by the time they reach the wall of the apparatus that they are essentially solid, and so that they form a fine powder and do not stick to the apparatus wall. The actual length of time to achieve this level of dryness depends on the size of the droplets. Droplet sizes generally range from 1 μm to 500 μm in diameter, with 5 to 100 μm being more typical. The large surface-to-volume ratio of the droplets and the large driving force for evaporation of solvent leads to actual drying times of a few seconds or less, and more typically less than 0.1 second. This rapid drying is often critical to the particles maintaining a uniform, homogeneous dispersion instead of separating into drug-rich and polymer-rich phases. Solidification times should be less than 100 seconds, preferably less than a few seconds, and more preferably less than 1 second. In general, to achieve this rapid solidification of the GPI/polymer solution, it is preferred that the size of droplets formed during the spray-drying process are less than 100 μm in diameter, preferably less than 50 μm in diameter, and more preferably less than 25 μm in diameter. The resultant solid particles thus formed are generally less than 100 μm in diameter, and preferably less than 50 μm in diameter, and more preferably less than 25 μm in diameter. Typically, particles are 1 to 20 μm in diameter.

Following solidification, the solid powder typically stays in the spray-drying chamber for about 5 to 60 seconds, further evaporating solvent from the solid powder. The final solvent content of the solid dispersion as it exits the dryer should be low, since this reduces the mobility of GPI molecules in the dispersion, thereby improving its stability. Generally, the solvent content of the dispersion as it leaves the spray-drying chamber should be less than 10 wt % and preferably less than 2 wt %. In some cases, it may be preferable to spray a solvent or a solution of a polymer or other excipient into the spray-drying chamber to form granules, so long as the dispersion is not adversely affected.

Spray-drying processes and spray-drying equipment are described generally in Perry's Chemical Engineers' Handbook, Sixth Edition (R. H. Perry, D. W. Green, J. O. Maloney, eds.) McGraw-Hill Book Co. 1984, pages 20-54 to 20-57. More details on spray-drying processes and equipment are reviewed by Marshall “Atomization and Spray-Drying,” 50 Chem. Eng. Prog. Monogr. Series 2 (1954).

Where the composition is a simple physical mixture, the composition may be prepared by dry- or wet-mixing the drug or drug mixture with the polymer to form the composition. Mixing processes include physical processing as well as wet-granulation and coating processes. Any conventional mixing method may be used, including those that substantially convert the drug and polymer to a molecular dispersion.

For example, mixing methods include convective mixing, shear mixing, or diffusive mixing. Convective mixing involves moving a relatively large mass of material from one part of a powder bed to another, by means of blades or paddles, revolving screw, or an inversion of the powder bed. Shear mixing occurs when slip planes are formed in the material to be mixed. Diffusive mixing involves an exchange of position by single particles. These mixing processes can be performed using equipment in batch or continuous mode. Tumbling mixers (e.g., twin-shell) are commonly used equipment for batch processing. Continuous mixing can be used to improve composition uniformity.

Milling may also be employed to prepare the compositions of the present invention. Milling is the mechanical process of reducing the particle size of solids (comminution). The most common types of milling equipment are the rotary cutter, the hammer, the roller and fluid energy mills. Equipment choice depends on the characteristics of the ingredients in the drug form (e.g., soft, abrasive, or friable). Wet- or dry-milling techniques can be chosen for several of these processes, also depending on the characteristics of the ingredients (e.g., drug stability in solvent). The milling process may serve simultaneously as a mixing process if the feed materials are heterogeneous. Conventional mixing and milling processes suitable for use in the present invention are discussed more fully in Lachman, et al., The Theory and Practice of Industrial Pharmacy (3d Ed. 1986). The components of the compositions of this invention may also be combined by dry- or wet-granulating processes.

In addition to the physical mixtures described above, the compositions of the present invention may constitute any device or collection of devices that accomplishes the objective of delivering to the use environment both the GPI and the concentration-enhancing polymer. Thus, in the case of oral administration to an animal, the dosage form may constitute a layered tablet wherein one or more layers comprise the amorphous GPI and one or more other layers comprise the polymer. Alternatively, the dosage form may be a coated tablet wherein the tablet core comprises the GPI and the coating comprises the concentration-enhancing polymer. In addition, the GPI and the polymer may even be present in different dosage forms such as tablets or beads and may be administered simultaneously or separately as long as both the GPI and polymer are administered in such a way that the GPI and polymer can come into contact in the use environment. When the GPI and the polymer are administered separately it is generally preferable to deliver the polymer prior to the GPI.

The amount of concentration-enhancing polymer relative to the amount of GPI present in the mixtures of the present invention depends on the GPI and polymer and may vary widely from a GPI-to-polymer weight ratio of from 0.01 to about 4 (e.g., 1 wt % GPI to 80 wt % GPI). However, in most cases it is preferred that the GPI-to-polymer ratio is greater than about 0.05 (4.8 wt % GPI) and less than about 2.5 (71 wt % GPI). Often the enhancement in GPI concentration or relative bioavailability that is observed increases as the GPI-to-polymer ratio decreases from a value of about 1 (50 wt % GPI) to a value of about 0.11 (10 wt % GPI). The maximum GPI:polymer ratio that yields satisfactory results varies from GPI to GPI and is best determined in in vitro dissolution tests and/or in vivo bioavailability tests. It should be noted that this level of concentration-enhancing polymer is usually substantially greater and often much greater than the amount of polymer conventionally included in dosage forms for other uses such as binders or coatings. Thus, it is preferred in the compositions of this invention that there be included sufficient concentration-enhancing polymer that the compositions meet the in vitro MDC and AUC criteria and in vivo bioavailability criterion previously set forth.

In general, to maximize the GPI concentration or relative bioavailability of the GPI, lower GPI-to-polymer ratios are preferred. At low GPI-to-polymer ratios, there is sufficient polymer available in solution to ensure the inhibition of the precipitation or crystallization of GPI from solution and, thus, the average concentration of GPI is much higher. For high GPI-to-polymer ratios, not enough polymer may be present in solution and GPI precipitation or crystallization of the GPI may occur more readily. In addition, the amount of concentration-enhancing polymer that can be used in a dosage form is often limited by the total mass requirements of the dosage form. For example, when oral dosing to a human is desired, at low GPI-to-polymer ratios the total mass of drug and polymer may be unacceptably large for delivery of the desired dose in a single tablet or capsule. Thus, it is often necessary to use GPI-to-polymer ratios that are less than optimum in specific dosage forms to provide a sufficient GPI dose in a dosage form that is small enough to be easily delivered to a use environment.

### EXCIPIENTS AND DOSAGE FORMS

Although the key ingredients present in the compositions of the present invention are simply the GPI to be delivered and the concentration-enhancing polymer(s), the inclusion of other excipients in the composition may be useful. These excipients may be utilized with the GPI/polymer mixture in order to formulate the mixture into tablets, capsules, suspensions, powders for suspension, creams, transdermal patches, depots, and the like. The amorphous GPI and polymer can be added to other dosage form ingredients in essentially any manner that does not substantially alter the GPI. In addition, as described above, the GPI and the polymer may be mixed with excipients separately to form different beads, or layers, or coatings, or cores or even separate dosage forms.

One very useful class of excipients is surfactants. Suitable surfactants include fatty acid and alkyl sulfonates; commercial surfactants such as benzalkonium chloride (HYAMINE® 1622, available from Lonza, Inc., Fairlawn, N.J.); dioctyl sodium sulfosuccinate, DOCUSATE SODIUM™ (available from Mallinckrodt Spec. Chem., St. Louis, MO.); polyoxyethylene sorbitan fatty acid esters (TWEEN®, available from ICI Americas Inc., Wilmington, Del.; LIPOSORB® P-20 available from Lipochem Inc., Patterson N.J.; CAPMUL® POE-0 available from Abitec Corp., Janesville, Wis.), and natural surfactants such as sodium taurocholic acid, 1-palmitoyl-2-oleoyl-sn-glycero-3-phosphocholine, lecithin, and other phospholipids and mono- and diglycerides. Such materials can advantageously be employed to increase the rate of dissolution by facilitating wetting, thereby increasing the maximum dissolved concentration, and also to inhibit crystallization or precipitation of drug by interacting with the dissolved drug by mechanisms such as complexation, formation of inclusion complexes, formation of micelles or adsorbing to the surface of solid drug, crystalline or amorphous. These surfactants may comprise up to 5 wt % of the composition.

The addition of pH modifiers such as acids, bases, or buffers may also be beneficial, retarding the dissolution of the composition (e.g., acids such as citric acid or succinic acid when the concentration-enhancing polymer is anionic) or, alternatively, enhancing the rate of dissolution of the composition (e.g., bases such as sodium acetate or amines when the polymer is anionic).

Conventional matrix materials, complexing agents, solubilizers, fillers, disintegrating agents (disintegrants), or binders may also be added as part of the composition itself or added by granulation via wet or mechanical or other means. These materials may comprise up to 90 wt % of the composition.

Examples of matrix materials, fillers, or diluents include lactose, mannitol, xylitol, microcrystalline cellulose, calcium diphosphate, and starch.

Examples of disintegrants include sodium starch glycolate, sodium alginate, carboxy methyl cellulose sodium, methyl cellulose, and croscarmellose sodium.

Examples of binders include methyl cellulose, microcrystalline cellulose, starch, and gums such as guar gum, and tragacanth.

Examples of lubricants include magnesium stearate and calcium stearate.

Other conventional excipients may be employed in the compositions of this invention, including those excipients well-known in the art. Generally, excipients such as pigments, lubricants, flavorants, and so forth may be used for customary purposes and in typical amounts without adversely affecting the properties of the compositions. These excipients may be utilized in order to formulate the composition into tablets, capsules, suspensions, powders for suspension, creams, transdermal patches, and the like.

Compositions of this invention may be used in a wide variety of dosage forms for administration of GPIs. Exemplary dosage forms are powders or granules that may be taken orally either dry or reconstituted by addition of water or other liquids to form a paste, slurry, suspension or solution; tablets; capsules; multiparticulates; and pills. Various additives may be mixed, ground, or granulated with the compositions of this invention to form a material suitable for the above dosage forms.

The compositions of the present invention may be formulated in various forms such that they are delivered as a suspension of particles in a liquid vehicle. Such suspensions may be formulated as a liquid or paste at the time of manufacture, or they may be formulated as a dry powder with a liquid, typically water, added at a later time but prior to oral administration. Such powders that are constituted into a suspension are often termed sachets or oral powder for constitution (OPC) formulations. Such dosage forms can be formulated and reconstituted via any known procedure. The simplest approach is to formulate the dosage form as a dry powder that is reconstituted by simply adding water and agitating. Alternatively, the dosage form may be formulated as a liquid and a dry powder that are combined and agitated to form the oral suspension. In yet another embodiment, the dosage form can be formulated as two powders which are reconstituted by first adding water to one powder to form a solution to which the second powder is combined with agitation to form the suspension.

Generally, it is preferred that the dispersion of GPI or amorphous form of GPI be formulated for long-term storage in the dry state as this promotes the chemical and physical stability of the GPI. Various excipients and additives are combined with the compositions of the present invention to form the dosage form. For example, it may be desirable to add some or all of the following: preservatives such as sulfites (an antioxidant), benzalkonium chloride, methyl paraben, propyl paraben, benzyl alcohol or sodium benzoate; suspending agents or thickeners such as xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, or titanium dioxide; anticaking agents or fillers such as silicon oxide, or lactose; flavorants such as natural or artificial flavors; sweeteners such as sugars such as sucrose, lactose, or sorbitol as well as artificial sweeteners such as aspartame or saccharin; wetting agents or surfactants such as various grades of polysorbate, docusate sodium, or sodium lauryl sulfate; solubilizers such as ethanol propylene glycol or polyethylene glycol; coloring agents such as FD and C Red No. 3 or FD and C Blue No. 1; and pH modifiers or buffers such as carboxylic acids (including citric acid, ascorbic acid, lactic acid, and succinic acid), various salts of carboxylic acids, amino acids such as glycine or alanine, various phosphate, sulfate and carbonate salts such as trisodium phosphate, sodium bicarbonate or potassium bisulfate, and bases such as amino glucose or triethanol amine.

A preferred additive to such formulations is additional concentration-enhancing polymer which may act as a thickener or suspending agent as well as to enhance the concentration of GPI in the environment of use and may also act to prevent or retard precipitation or crystallization of GPI from solution. Such preferred additives are hydroxyethyl cellulose, hydroxypropyl cellulose, and hydroxypropyl methyl cellulose. In particular, the salts of carboxylic acid functional polymers such as cellulose acetate phthalate, hydroxypropyl methyl cellulose acetate succinate, and carboxymethyl cellulose are useful in this regard. Such polymers may be added in their salt forms or the salt form may be formed in situ during reconstitution by adding a base such as trisodium phosphate and the acid form of such polymers.

In some cases, the overall dosage form or particles, granules or beads that make up the dosage form may have superior performance if coated with an enteric polymer to prevent or retard dissolution until the dosage form leaves the stomach. Exemplary enteric coating materials include hydroxypropyl methyl cellulose acetate succinate, hydroxypropyl methyl cellulose phthalate, cellulose acetate phthalate, cellulose acetate trimellitate, carboxylic acid-functionalized polymethacrylates, and carboxylic acid-functionalized polyacrylate.

Compositions of this invention may be administered in a controlled release dosage form. In one such dosage form, the composition of the GPI and polymer is incorporated into an erodible polymeric matrix device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or “matrix” that entraps the mixture of GPI and polymer. The aqueous-swollen matrix gradually erodes, swells, disintegrates or dissolves in the environment of use, thereby controlling the release of the drug mixture to the environment of use. Examples of such dosage forms are disclosed more fully in commonly assigned pending U.S. patent application Ser. No. 09/495,059 filed Jan. 31, 2000 which claimed the benefit of priority of provisional patent application Ser. No. 60/119,400 filed Feb. 10, 1999, the relevant disclosure of which is herein incorporated by reference.

Alternatively, the compositions of the present invention may be administered by or incorporated into a non-erodible matrix device.

Alternatively, the drug mixture of the invention may be delivered using a coated osmotic controlled release dosage form. This dosage form has two components: (a) the core which contains an osmotic agent and the GPI and the concentration-enhancing polymer; and (b) a non-dissolving and non-eroding coating surrounding the core, the coating controlling the influx of water to the core from an aqueous environment of use so as to cause drug release by extrusion of some or all of the core to the environment of use. The GPI and the concentration-enhancing polymer may be homogeneously distributed throughout the core or they may be partially or completely segregated in separate regions of the core. The osmotic agent contained in the core of this device may be an aqueous-swellable hydrophilic polymer, osmogen, or osmaqent. The coating is preferably polymeric, aqueous-permeable, and has at least one delivery port. Examples of such dosage forms are disclosed more fully in commonly assigned pending U.S. patent application Ser. No. 09/495,061 filed Jan. 31, 2000 which claimed the benefit of priority of provisional Patent Application Ser. No. 60/119,406 filed Feb. 10, 1999, the relevant disclosure of which is herein incorporated by reference.

Alternatively, the drug mixture of the invention may be delivered via a coated hydrogel controlled release dosage form having at least three components: (a) a composition containing the GPI, (b) a water-swellable composition wherein the water-swellable composition is in a separate region within a core formed by the drug-containing composition and the water-swellable composition, and (c) a coating around the core that is water-permeable, water-insoluble, and has a least one delivery port therethrough. In use, the core imbibes water through the coating, swelling the water-swellable composition and increasing the pressure within the core, and fluidizing the GPI-containing composition. Because the coating remains intact, the GPI-containing composition is extruded out of the delivery port into an environment of use. The polymer may be delivered in a separate dosage form, may be included in the GPI-containing composition, may comprise a separate composition that occupies a separate region within the core, or may constitute all or part of a coating applied to the dosage form. Examples of such dosage forms are more fully disclosed in commonly assigned pending Provisional Application Ser. No. 60/171,968 filed Dec. 23, 1999, the relevant disclosure of which is herein iincorporated by reference.

Alternatively, the compositions may be administered as multiparticulates. Multiparticulates generally refer to dosage forms that comprise a multiplicity of particles that may range in size from about 10 μm to about 2 mm, more typically about 100 μm to 1 mm in diameter. Such multiparticulates may be packaged, for example, in a capsule such as a gelatin capsule or a capsule formed from an aqueous-soluble polymer such as HPMCAS, HPMC or starch or they may be dosed as a suspension or slurry in a liquid. Such particulates may be made by any known process such as wet and dry granulation processes or melt congeal processes such as those previously described for forming amorphous GPI. For example, the GPI and a glyceride such as hydrogenated vegetable oil, a vegetable or synthetic fat or a wax such as paraffin may be blended and fed to a melt congeal process as a solid or liquid, followed by cooling to form beads comprised of amorphous GPI and the excipient.

The so-formed beads may then be blended with one or more concentration-enhancing polymers with or without additional excipients to form a multiparticulate dosage form. Alternatively, a high melting point concentration-enhancing polymer such as HPMCAS may be blended with the GPI and the fat or wax fed as a solid blend to a melt congeal process or the blend may be heated such that the GPI and the fat or wax melt to form a slurry of concentration-enhancing polymer particles in molten GPI and fat or wax. The resulting material comprises beads or particles consisting of an amorphous dispersion of GPI in the fat or wax with concentration-enhancing polymer particles trapped therein. Alternatively, a dispersion of the GPI in a concentration-enhancing polymer may be blended with a fat or wax and then fed to a melt congeal process as a solid or a slurry of the dispersion in the molten fat or wax. Such processing yields particles or beads consisting of particles of dispersion trapped in the solidified fat or wax matrix.

Similar multiparticulate dosage forms may be made with the various compositions of this invention but using excipients suited to the bead-forming or granule-forming process chosen. For example, when granules are formed by extrusion/spheronization processes the dispersion or other composition may be blended with, for example, microcrystalline cellulose or other cellulosic polymer to aid in processing.

In any case, the resulting particles may themselves constitute the multiparticulate dosage form or they may be coated by various film-forming materials such as enteric polymers or water-swellable or water-soluble polymers, or they may be combined with other excipients or vehicles to aid in dosing to patients.

Alternatively, the compositions of the present invention may be co-administered, meaning that the GPI can be administered separately from, but within the same general time frame as, the polymer. Thus, amorphous GPI can, for example, be administered in its own dosage form which is taken at approximately the same time as the polymer which is in a separate dosage form. If administered separately, it is generally preferred to administer both the GPI and the polymer within 60 minutes, more preferably within 15 minutes, of each other, so that the two are present together in the environment of use. When not administered simultaneously, the polymer is preferably administered prior to the amorphous GPI.

In addition to the above additives or excipients, use of any conventional materials and procedures for preparation of suitable dosage forms using the compositions of this invention known by those skilled in the art are potentially useful.

In another aspect, the present invention concerns the treatment of diabetes, including impaired glucose tolerance, insulin resistance, insulin dependent diabetes mellitus (Type 1) and non-insulin dependent diabetes mellitus (NIDDM or Type 2). Also included in the treatment of diabetes are the treatment of the diabetic complications, such as neuropathy, nephropathy, retinopathy or cataracts. The compositions of the present invention can also be used for diabetes prevention.

Diabetes can be treated by administering to a patient having diabetes (Type 1 or Type 2), insulin resistance, impaired glucose tolerance, or any of the diabetic complications such as neuropathy, nephropathy, retinopathy or cataracts, a therapeutically effective amount of a composition of the present invention. It is also contemplated that diabetes be treated by administering a composition of the present invention in combination with other agents that can be used to treat diabetes.

Representative agents that can be used to treat diabetes include insulin and insulin analogs (e.g. LysPro insulin); GLP-1 (7-37) (insulinotropin) and GLP-1 (7-36)-NH₂; sulfonylureas and analogs: chlorpropamide, glibenclamide, tolbutamide, tolazamide, acetohexamide, glypizide, glimepiride, repaglinide, meglitinide; biguanides: metformin, phenformin, buformin; α2-antagonists and imidazolines: midaglizole, isaglidole, deriglidole, idazoxan, efaroxan, fluparoxan; Other insulin secretagogues: linogliride, A-4166; glitazones: ciglitazone, pioglitazone, englitazone, troglitazone, darglitazone, rosiglitazone; PPAR-gamma agonists; fatty acid oxidation inhibitors: clomoxir, etomoxir; α-glucosidase inhibitors: acarbose, miglitol, emiglitate, voglibose, MDL-25,637, camiglibose, MDL-73,945; β-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine; vanadate and vanadium complexes (e.g. Naglivan®) and peroxovanadium complexes; amylin antagonists; glucagon antagonists; gluconeogenesis inhibitors; somatostatin analogs and antagonists; antilipolytic agents: nicotinic acid, acipimox, WAG 994. Any combination of agents can be administered as described above.

In addition to the categories and compounds mentioned above, the compositions of the present invention can be administered in combination with thyromimetic compounds, aldose reductase inhibitors, glucocorticoid receptor antagonists, NHE-1 inhibitors, or sorbitol dehydrogenase inhibitors, or combinations thereof, to treat or prevent diabetes, insulin resistance, diabetic neuropathy, diabetic nephropathy, diabetic retinopathy, cataracts, hyperglycemia, hypercholesterolemia, hypertension, hyperinsulinemia, hyperlipidemia, atherosclerosis, or tissue ischemia, particularly myocardial ischemia.

It is generally accepted that thyroid hormones, specifically, biologically active iodothyronines, are critical to normal development and to maintaining metabolic homeostasis. Thyroid hormones stimulate the metabolism of cholesterol to bile acids and enhance the lipolytic responses of fat cells to other hormones. U.S. Pat. Nos. 4,766,121; 4,826,876; 4,910,305; and 5,061,798 disclose certain thyroid hormone mimetics (thyromimetics), namely, 3,5-dibromo-3′-[6-oxo-3(1H)-pyridazinylmethyl]-thyronines. U.S. Pat. No. 5,284,971 discloses certain thyromimetic cholesterol lowering agents, namely, 4-(3-cyclohexyl-4-hydroxy or -methoxy phenylsulfonyl)-3,5 dibromo-phenylacetic compounds. U.S. Pat. Nos. 5,401,772; 5,654,468; and 5,569,674 disclose certain thyromimetics that are lipid lowering agents, namely, heteroacetic acid derivatives. In addition, certain oxamic acid derivatives of thyroid hormones are known in the art. For example, N. Yokoyama, et al. in an article published in the Journal of Medicinal Chemistry, 38 (4): 695-707 (1995) describe replacing a —CH₂ group in a naturally occurring metabolite of T₃ with an —NH group resulting in —HNCOCO₂H. Likewise, R. E. Steele et al. in an article published in International Congressional Service (Atherosclerosis X) 1066: 321-324 (1995) and Z. F. Stephan et al. in an article published in Atherosclerosis, 126: 53-63 (1996), describe certain oxamic acid derivatives useful as lipid-lowering thyromimetic agents, yet devoid of undesirable cardiac activities.

Each of the thyromimetic compounds referenced above and other thyromimetic compounds can be used in combination with the compositions of the present invention to treat or prevent diabetes, insulin resistance, diabetic neuropathy, diabetic nephropathy, diabetic retinopathy, cataracts, hyperglycemia, hypercholesterolemia, hypertension, hyperinsulinemia, hyperlipidemia, atherosclerosis, or tissue ischemia.

The compositions of the present invention can also be used in combination with aldose reductase inhibitors. Aldose reductase inhibitors constitute a class of compounds that have become widely known for their utility in preventing and treating conditions arising from complications of diabetes, such as diabetic neuropathy and nephropathy. Such compounds are well known to those skilled in the art and are readily identified by standard biological tests. For example, the aldose reductase inhibitors zopolrestat, 1-phthalazineacetic acid, 3,4-dihydro-4-oxo-3-[[5-(trifluoromethyl)-2-benzothiazolyl]methyl]-, and related compounds are described in U.S. Pat. No. 4,939,140 to Larson et al.

Aldose reductase inhibitors have been taught for use in lowering lipid levels in mammals. See, for example, U.S. Pat. No. 4,492,706 to Kallai-sanfacon and EP 0 310 931 A2 (Ethyl Corporation).

U.S. Pat. No. 5,064,830 to Going discloses the use of certain oxophthalazinyl acetic acid aldose reductase inhibitors, including zopolrestat, for lowering of blood uric acid levels.

Commonly assigned U.S. Pat. No. 5,391,551 discloses the use of certain aldose reductase inhibitors, including zopolrestat, for lowering blood lipid levels in humans. The disclosure teaches that therapeutic utilities derive from the treatment of diseases caused by an increased level of triglycerides in the blood, such diseases include cardiovascular disorders such as thrombosis, arteriosclerosis, myocardial infarction, and angina pectoris. A preferred aldose reductase inhibitor is 1-phthalazineacetic acid, 3,4-dihydro-4-oxo-3-[[5-trifluoromethyl)-2-benzothiazolyl]methyl]-, also known as zopolrestat.

The term aldose reductase inhibitor refers to compounds that inhibit the bioconversion of glucose to sorbitol, which is catalyzed by the enzyme aldose reductase.

Any aldose reductase inhibitor may be used in a combination with a composition of the present invention. Aldose reductase inhibition is readily determined by those skilled in the art according to standard assays (J. Malone, Diabetes, 29:861-864 (1980). “Red Cell Sorbitol, an Indicator of Diabetic Control”). A variety of aldose reductase inhibitors are described herein; however, other aldose reductase inhibitors useful in the compositions and methods of this invention will be known to those skilled in the art.

The activity of an aldose reductase inhibitor in a tissue can be determined by testing the amount of aldose reductase inhibitor that is required to lower tissue sorbitol (i.e., by inhibiting the further production of sorbitol consequent to blocking aldose reductase) or lower tissue fructose (by inhibiting the production of sorbitol consequent to blocking aldose reductase and consequently the production of fructose.

Accordingly, examples of aldose reductase inhibitors useful in the compositions, combinations and methods of the present invention include:

1. 3-(4-bromo-2-fluorobenzyl)-3,4-dihydro-4-oxo-1-phthalazineacetic acid (ponalrestat, U.S. Pat. No. 4,251,528);

2. N[[(5-trifluoromethyl)-6-methoxy-1-naphthalenyl]thioxomethyl]-N-methylglycine (tolrestat, U.S. Pat. No. 4,600,724);

3. 5-[(Z,E)-β-methylcinnamylidene]-4-oxo-2-thioxo-3-thiazolideneacetic acid (epalrestat, U.S. Pat. No. 4,464,382, U.S. 4,791,126, U.S. 4,831,045);

4. 3-(4-bromo-2-fluorobenzyl)-7-chloro-3,4-dihydro-2,4-dioxo-1(2H)-quinazolineacetic acid (zenarestat, U.S. Pat. Nos. 4,734,419, and 4,883,800);

5. 2R,4R-6,7-dichloro-4-hydroxy-2-methylchroman-4-acetic acid (U.S. Pat. No. 4,883,410);

6. 2R,4R-6,7-dichloro-6-fluoro-4-hydroxy-2-methylchroman-4-acetic acid (U.S. Pat. No. 4,883,410);

7. 3,4-dihydro-2,8-diisopropyl-3-oxo-2H-1,4-benzoxazine-4-acetic acid (U.S. Pat. No. 4,771,050);

8. 3,4-dihydro-3-oxo-4-[(4,5,7-trifluoro-2-benzothiazolyl)methyl]-2H-1,4-benzothiazine-2-acetic acid (SPR-210, U.S. Pat. No. 5,252,572);

9. N-[3,5-dimethyl-4-[(nitromethyl)sulfonyl]phenyl]-2-methyl-benzeneacetamide (ZD5522, U.S. Pat. No. 5,270,342 and U.S. 5,430,060);

10. (S)-6-fluorospiro[chroman-4,4′-imidazolidine]-2,5′-dione (sorbinil, U.S. Pat. No. 4,130,714);

11. d-2-methyl-6-fluoro-spiro(chroman-4′,4′-imidazolidine)-2′,5′-dione (U.S. Pat. No. 4,540,704);

12. 2-fluoro-spiro(9H-fluorene-9,4′-imidazolidine)2′,5′-dione (U.S. Pat. No. 4,438,272);

13. 2,7-di-fluoro-spiro(9H-fluorene-9,4′-imidazolidine)2′,5′-dione (U.S. Pat. No. 4,436,745, U.S. 4,438,272);

14. 2,7-di-fluoro-5-methoxy-spiro(9H-fluorene-9,4′-imidazolidine)2′,5′-dione (U.S. Pat. No. 4,436,745, U.S. 4,438,272);

15. 7-fluoro-spiro(5H-indenol[1,2-b]pyridine-5,3′-pyrrolidine)2,5′-dione (U.S. Pat. No. 4,436,745, U.S. 4,438,272);

16. d-cis-6′-chloro-2′,3′-dihydro-2′-methyl-spiro-(imidazolidine-4,4′-4′-H-pyrano(2,3-b)pyridine)-2,5-dione (U.S. Pat. No. 4,980,357);

17. spiro imidazolidine-4,5′(6H)-quinoline]2,5-dione-3′-chloro-7,′8′-dihydro-7′-methyl-(5′-cis)(U.S. Pat. No. 5,066,659);

18. (2S,4S)-6-fluoro-2′,5′-dioxospiro(chroman-4,4′-imidazolidine)-2-carboxamide (U.S. Pat. No. 5,447,946); and

19. 2-[(4-bromo-2-fluorophenyl)methyl]-6-fluorospiro[isoquinoline-4(1H),3′-pyrrolidine]-1,2′,3,5′(2H)-tetrone (ARI-509, U.S. Pat. No. 5,037,831).

Other aldose reductase inhibitors include compounds having Formula Ia below

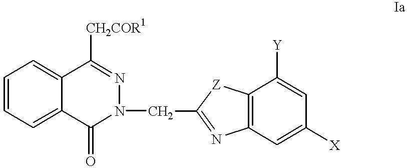

or a pharmaceutically acceptable salt or prodrug thereof, wherein the substituents of Formula Ia are as follows:

Z is O or S;

R¹ is hydroxy or a group capable of being removed in vivo to produce a compound of Formula I wherein R¹ is OH; and

X and Y are the same or different and are selected from hydrogen, trifluoromethyl, fluoro, and chloro.

A preferred subgroup within the above group of aldose reductase inhibitors includes numbered compounds 1, 2, 3, 4, 5, 6, 9, 10, and 17, and the following compounds of Formula Ia:

20. 3,4-dihydro-3-(5-fluorobenzothiazol-2-ylmethyl)-4-oxophthalazin-1-yl-acetic acid [R¹=hydroxy; X=F; Y=H];

21. 3-(5,7-difluorobenzothiazol-2-ylmethyl)-3,4-dihydro-4-oxophthalazin-1-ylacetic acid [R¹=hydroxy; X=Y=F];

22. 3-(5-chlorobenzothiazol-2-ylmethyl)-3,4-dihydro-4-oxophthalazin-1-ylacetic acid [R¹=hydroxy; X=Cl; Y=H];

23. 3-(5,7-dichlorobenzothiazol-2-ylmethyl)-3,4-dihydro-4-oxophthalazin-1-ylacetic acid [R¹=hydroxy; X=Y=Cl];

24. 3,4-dihydro-4-oxo-3-(5-trifluoromethylbenzoxazol-2-ylmethyl)phthalazin-1-ylacetic acid [R¹=hydroxy; X=CF₃; Y=H];

25. 3,4-dihydro-3-(5-fluorobenzoxazol-2-ylmethyl)-4-oxophthalazin-1-yl-acetic acid [R¹=hydroxy; X=F; Y=H];

26. 3-(5,7-difluorobenzoxazol-2-ylmethyl)-3,4-dihydro-4-oxophthalazin-1-ylacetic acid [R¹=hydroxy; X=Y=F];

27. 3-(5-chlorobenzoxazol-2-ylmethyl)-3,4-dihydro-4-oxophthalazin-1-ylacetic acid [R¹=hydroxy; X=Cl; Y=H];

28. 3-(5,7-dichlorobenzoxazol-2-ylmethyl)-3,4-dihydro-4-oxophthalazin-1-ylacetic acid [R¹=hydroxy; X=Y=Cl]; and

29. zopolrestat; 1-phthalazineacetic acid, 3,4-dihydro-4-oxo-3-[[5-(trifluoromethyl)-2-benzothiazolyl]methyl]-[R¹=hydroxy; X=trifluoromethyl; Y=H].

In compounds 20-23, and 29 Z is S. In compounds 24-28, Z is O.

Of the above subgroup, compounds 20-29 are more preferred with 29 especially preferred. Procedures for making the aldose reducatase inhibitors of formula Ia can be found in PCT publication number WO 99/26659.

Each of the aldose reductase inhibitors referenced above and other aldose reductase inhibitors can be used in combination with the compounds of the present invention to treat diabetes, insulin resistance, diabetic neuropathy, diabetic nephropathy, diabetic retinopathy, cataracts, hyperglycemia, hypercholesterolemia, hypertension, hyperinsulinemia, hyperlipidemia, atherosclerosis, or tissue ischemia.

The compositions of the present invention can also be used in combination with glucocorticoid receptor antagonists. The glucocorticoid receptor (GR) is present in glucocorticoid responsive cells where it resides in the cytosol in an inactive state until it is stimulated by an agonist. Upon stimulation the glucocorticoid receptor translocates to the cell nucleus where it specifically interacts with DNA and/or protein(s) and regulates transcription in a glucocorticoid responsive manner. Two examples of proteins that interact with the glucocorticoid receptor are the transcription factors, API and NFκ-B. Such interactions result in inhibition of API- and NFκ-B-mediated transcription and are believed to be responsible for the anti-inflammatory activity of endogenously administered glucocorticoids. In addition, glucocorticoids may also exert physiologic effects independent of nuclear transcription. Biologically relevant glucocorticoid receptor agonists include cortisol and corticosterone. Many synthetic glucocorticoid receptor agonists exist including dexamethasone, prednisone and prednisilone. By definition, glucocorticoid receptor antagonists bind to the receptor and prevent glucocorticoid receptor agonists from binding and eliciting GR mediated events, including transcription. RU486 is an example of a non-selective glucocorticoid receptor antagonist. GR antagonists can be used in the treatment of diseases associated with an excess or a deficiency of glucocorticoids in the body. As such, they may be used to treat the following: obesity, diabetes, cardiovascular disease, hypertension, Syndrome X, depression, anxiety, glaucoma, human immunodeficiency virus (HIV) or acquired immunodeficiency syndrome (AIDS), neurodegeneration (for example, Alzheimer's and Parkinson's), cognition enhancement, Cushing's Syndrome, Addison's Disease, osteoporosis, frailty, inflammatory diseases (such as osteoarthritis, rheumatoid arthritis, asthma and rhinitis), tests of adrenal function, viral infection, immunodeficiency, immunomodulation, autoimmune diseases, allergies, wound healing, compulsive behavior, multi-drug resistance, addiction, psychosis, anorexia, cachexia, post-traumatic stress syndrome, post-surgical bone fracture, medical catabolism and prevention of muscle frailty. Examples or GR antagonists that can be used in combination with a compound of the present invention include compounds of Formula Ib below:

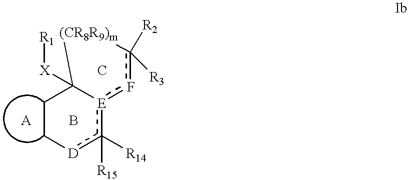

an isomer thereof, a prodrug of said compound or isomer, or a pharmaceutically acceptable salt of said compound, isomer or prodrug wherein the substituents of Formula Ib are as follows:

m is 1 or 2;

- - - represents an optional bond;

A is selected from the group consisting of

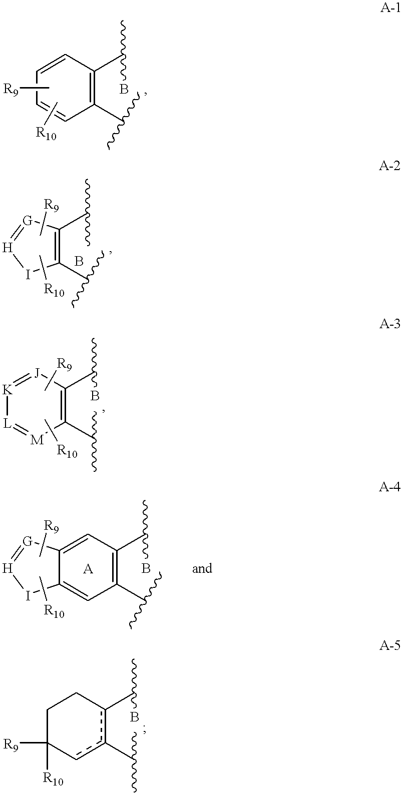

D is CR₇, CR₇R₁₆, N, NR₇ or O;

E is C, CR₆ or N;

F is CR₄, CR₄R₅ or O;

G, H and I together with 2 carbon atoms from the A-ring or 2 carbon atoms from the B-ring form a 5-membered heterocyclic ring comprising one or more N, O or S atoms; provided that there is at most one of O and S per ring;

J, K, L and M together with 2 carbon atoms from the B-ring forms a 6-membered heterocyclic ring comprising 1 or more N atoms;

X is a) absent, b) —CH₂—, c) —CH(OH)— or d) —C(O)—;

R₁ is a) —H, b) —Z—CF₃, c) —(C₁-C₆)alkyl, d) —(C₂-C₆)alkenyl, e) —(C₂-C₆)alkynyl, f) —CHO, g) —CH═N—OR₁₂, h) —Z—C(O)OR₁₂, i) —Z—C(O)—NR₁₂R₁₃, j) —Z—C(O)—NR₁₂—Z-het, k) —Z—NR₁₂R₁₃, l) —Z—NR₁₂het, m) —Z-het, n) —Z—O-het, o) —Z-aryl′, p) —Z—O-aryl′, q) —CHOH-aryl′ or r) —C(O)-aryl′ wherein aryl′ in substituents o) to r) is substituted independently with 0, 1 or 2 of the following: —Z—OH, —Z—NR₁₂R₁₃, —Z—NR₁₂-het, —C(O)NR₁₂R₁₃, —C(O)O(C₁-C₆)alkyl, —C(O)OH, —C(O)-het, —NR₁₂—C(O)—(C₁-C₆)alkyl, —NR₁₂—C(O)—(C₂-C₆)alkenyl, —NR₁₂—C(O)—(C₂-C₆)alkynyl, —NR₁₂—C(O)—Z-het, —CN, —Z-het, —O—(C₁-C₃)alkyl-C(O)—NR₁₂R₁₃, —O—(C₁-C₃)alkyl-C(O)O(C₁-C₆)alkyl, —NR₁₂—Z—C(O)O(C₁-C₆)alkyl, —N(Z—C(O)O(C₁-C₆)alkyl)₂, —NR₁₂—Z—C(O)—NR₁₂R₁₃, —Z—NR₁₂—SO₂—R₁₃, —NR₁₂—SO₂-het, —C(O)H, —Z—NR₁₂—Z—O(C₁-C₆) alkyl, —Z—NR₁₂—Z—NR₁₂R₁₃, —Z—NR₁₂-(C₃-C₆)cycloalkyl, —Z—N(Z—O(C₁-C₆)alkyl)₂, —SO₂R₁₂, —SOR₁₂, —SR₁₂, —SO₂NR₁₂R₁₃, —O—C(O)—(C₁-C₄)alkyl, —O—SO₂—(C₁-C₄)alkyl, -halo or —CF₃;

Z for each occurrence is independently a) —(C₀-C₆)alkyl, b) —(C₂-C₆)alkenyl or c) —(C₂-C₆)alkynyl;

R₂ is a) —H, b) -halo, c) —OH, d) -—(C₁-C₆) alkyl substituted with 0 or 1—OH, e) —NR₁₂R₁₃, f) —Z—C(O)O(C₁-C₆)alkyl, g) —Z—C(O)NR₁₂R₁₃, h) —O—(C₁-C₆)alkyl, i) —Z—O—C(O)—(C₁-C₆)alkyl, j) —Z—O—(C₁-C₃) alkyl-C(O)—NR₁₂R₁₃, k) —Z—O—(C₁-C₃)alkyl-C(O)—O(C₁-C₆)alkyl, l) —O—(C₂-C₆)alkenyl, m) —O—(C₂-C₆)alkynyl, n) —O—Z-het, o) —COOH, p) —C(OH)R₁₂R₁₃ or q) —Z—CN;

R₃ is a) —H, b) —(C₁-C₁₀)alkyl wherein 1 or 2 carbon atoms, other than the connecting carbon atom, may optionally be replaced with 1 or 2 heteroatoms independently selected from S, O and N and wherein each carbon atom is substituted with 0, 1 or 2 R_{y}, c) —(C₂-C₁₀)alkenyl substituted with 0, 1 or 2 R_{y}, d) —(C₂-C₁₀)alkynyl wherein 1 carbon atom, other than the connecting carbon atom, may optionally be replaced with 1 oxygen atom and wherein each carbon atom is substituted with 0, 1 or 2 R_{y}, e) —CH═C═CH₂, f) —CN, g) —(C₃-C₆)cycloalkyl, h) —Z-aryl, i) —Z-het, j) —C(O)O(C₁-C₆)alkyl, k) —O(C₁-C₆)alkyl, 1)-Z-S-R₁₂, m) —Z—S(O)—R₁₂, n) —Z—S(O)₂—R₁₂, o) —CF₃ p) —NR₁₂O—(C₁-C₆)alkyl or q) —CH₂OR_{y};

provided that one of R₂ and R₃ is absent when there is a double bond between CR₂R₃ (the 7 position) and the F moiety (the 8 position) of the C-ring;

R_{y} for each occurrence is independently a) —OH, b) -halo, c) —Z—CF₃, d) —Z—CF(C₁-C₃ alkyl)₂, e) —CN, f) —NR₁₂R₁₃, q) —(C₃-C₆)cycloalkyl, h) —(C₃-C₆) cycloalkenyl, i) —(C₀-C₃)alkyl-aryl, j) -het or k) —N₃;

or R₂ and R₃ are taken together to form a) ═CHR₁₁, b) ═NOR₁₁, c) ═O, d) ═N—NR₁₂, e) ═N—NR₁₂—C(O)—R₁₂, f) oxiranyl or g) 1,3-dioxolan-4-yl;

R₄ and R₅ for each occurrence are independently a) —H, b) —CN, c) —(C₁-C₆)alkyl substituted with 0 to 3 halo, d) —(C₂-C₆)alkenyl substituted with 0 to 3 halo, e) —(C₂-C₆)alkynyl substituted with 0 to 3 halo, f) —O—(C₁-C₆)alkyl substituted with 0 to 3 halo, g) —O—(C₂-C₆)alkenyl substituted with 0 to 3 halo, h) —O—(C₂-C₆)alkynyl substituted with 0 to 3 halo, i) halo, j) —OH, k) (C₃-C₆)cycloalkyl or l) (C₃-C₆)cycloalkenyl;

or R₄ and R₅ are taken together to form ═O;

R₆ is a) —H, b) —CN, c) —(C₁-C₆)alkyl substituted with 0 to 3 halo, d) —(C₂-C₆)alkenyl substituted with 0 to 3 halo, e) —(C₂-C₆)alkynyl substituted with 0 to 3 halo or f) —OH;

R₇ and R₁₆ for each occurrence are independently a) —H, b) -halo, c) —CN, d) —(C₁-C₆)alkyl substituted with 0 to 3 halo, e) —(C₂-C₆)alkenyl substituted with 0 to 3 halo or f) —(C₂-C₆)alkynyl substituted with 0 to 3 halo; provided that R₇ is other than —CN or -halo when D is NR₇;

or R₇ and R₁₆ are taken together to form ═O;

R₈, R₉, R₁₄ and R₁₅ for each occurrence are independently a) —H, b) -halo, c) (C₁-C₆)alkyl substituted with 0 to 3 halo, d) —(C₂-C)alkenyl substituted with 0 to 3 halo, e) —(C₂-C₆)alkynyl substituted with 0 to 3 halo, f) —CN, g) —(C₃-C₆)cycloalkyl, h) —(C₃-C₆)cycloalkenyl, i) —OH, j) —O—(C₁-C₆)alkyl, k) —O—(C₁-C₆)alkenyl, l) —O—(C₁-C₆)alkynyl, m) —NR₁₂R₁₃, n) —C(O)OR₁₂ or o) —C(O)NR₁₂R₁₃;

or R₈ and R₉ are taken together on the C-ring to form ═O; provided that when m is 2, only one set of R₈ and R₉ are taken together to form ═O;

or R₁₄ and R₁₅ are taken together to form ═O; provided that when R₁₄ and R₁₅ are taken together to form ═O, D is other than CR₇ and E is other than C;

R₁₀ is a) —(C₁-C₁₀)alkyl substituted with 0 to 3 substituents independently selected from -halo, —OH and —N₃, b) —(C₂-C₁₀)alkenyl substituted with 0 to 3 substituents independently selected from -halo, —OH and —N₃, c) —(C₂-C₁₀)alkynyl substituted with 0 to 3 substituents independently selected from -halo, —OH and —N₃, d) -halo, e) —Z—CN, f) —OH, g) —Z-het, h) —Z—NR₁₂R₁₃, i) —Z—C(O)-het, j) —Z—C(O)—(C₁-C₆)alkyl, k) —Z—C(O)—NR₁₂R₁₃, l) —Z—C(O)—NR₁₂—Z—CN, m) —Z—C(O)—NR₁₂—Z-het, n) —Z—C(O)—NR₁₂—Z-aryl, o) —Z—C(O)—NR₁₂—Z—NR₁₂R₁₃, p) —Z—C(O)—NR₁₂—Z—O(C₁-C₆) alkyl, q) —(C₁-C₆) alkyl-C(O)OH, r) —Z—C(O)O(C₁-C₆)alkyl, s) —Z—O—(C₀-C₆)alkyl-het, t) —Z—O—(C₀-C₆)alkyl-aryl, u) —Z—O—(C₁-C₆)alkyl substituted with 0 to 2 Rₓ, v) —Z—O—(C₁-C₆)alkyl-CH(O), w) —Z—O—(C₁-C₆)alkyl-NR₁₂-het, x) —Z—O—Z-het-Z-het, y) —Z—O—Z-het-Z—NR₁₂R₁₃, z) —Z—O—Z-het-C(O)-het, a1) —Z—O—Z—C(O)-het, b1) —Z—O—Z—C(O)-het-het, c1) —Z—O—Z—C(O)—(C₁-C₆)alkyl, d1) —Z—O—Z—C(S)—NR₁₂R₁₃, e1) —Z—O—Z—C(O)—NR₁₂R₁₃, f1) —Z—O—Z—(C₁-C₃)alkyl-C(O)—NR₁₂R₁₃, g1) —Z—O—Z—C(O)—O(C₁-C₆)alkyl, h1) —Z—O—Z—C(O)—OH, i1) —Z—O—Z—C(O)—NR₁₂—O(C₁-C₆)alkyl, j1) —Z—O—Z—C(O)—NR₁₂—OH, k1) —Z—O—Z—C(O)—NR₁₂—Z—NR₁₂R₃, l1) —Z—O—Z—C(O)—NR₁₂—Z-het, m1) —Z—O—Z—C(O)—NR₁₂—SO₂—(C₁-C₆)alkyl, n1) —Z—O—Z—C(═NR₁₂)(NR₁₂R₁₃), o1) —Z—O—Z—C(═NOR₁₂)(NR₁₂R₁₃) p1) —Z—NR₁₂—C(O)—O—Z—NR₁₂R₁₃, q1) —Z—S—C(O)—NR₁₂R₁₃, r1) —Z—O—SO₂—(C₁-C₆)alkyl, s1) —Z—O—SO₂-aryl, t1) —Z—O—SO₂—NR₁₂R₁₃, u1) —Z—O—SO₂—CF₃, v1) —Z—NR₁₂C(O)OR₁₃ or w1) —Z—NR₁₂C(O)R₁₃;

or R₉ and R₁₀ are taken together on the moiety of formula A-5 to form a) ═O or b) ═NOR₁₂;

R₁₁ is a) —H, b) —(C₁-C₅) alkyl, c) —(C₃-C₆)cycloalkyl or d) —(C₀-C₃)alkyl-aryl;

R₁₂ and R₁₃ for each occurrence are each independently a) —H, b) —(C₁-C₆)alkyl wherein 1 or 2 carbon atoms, other than the connecting carbon atom, may optionally be replaced with 1 or 2 heteroatoms independently selected from S, O and N and wherein each carbon atom is substituted with 0 to 6 halo, c) —(C₂-C₆)alkenyl substituted with 0 to 6 halo or d) —(C₁-C₆)alkynyl wherein 1 carbon atom, other than the connecting carbon atom, may optionally be replaced with 1 oxygen atom and wherein each carbon atom is substituted with 0 to 6 halo;

or R₁₂ and R₁₃ are taken together with N to form het;

or R₆ and R₁₄ or R₁₅ are taken together to form 1,3-dioxolanyl;

aryl is a) phenyl substituted with 0 to 3 Rₓ, b) naphthyl substituted with 0 to 3 Rₓ or c) biphenyl substituted with 0 to 3 Rₓ;

het is a 5-,6- or 7-membered saturated, partially saturated or unsaturated ring containing from one (1) to three (3) heteroatoms independently selected from the group consisting of nitrogen, oxygen and sulfur; and including any bicyclic group in which any of the above heterocyclic rings is fused to a benzene ring or another heterocycle; and the nitrogen may be in the oxidized state giving the N-oxide form; and substituted with 0 to 3 Rₓ;

Rₓ for each occurrence is independently a) -halo, b) —OH, c) —(C₁-C₆)alkyl, d) —(C₂-C₆)alkenyl, e) —(C₂-C₆)alkynyl, f) —O(C₁-C₆)alkyl, g) —O(C₂-C₆)alkenyl, h) —O(C₂-C₆)alkynyl, i) —(C₀-C₆)alkyl-NR₁₂R₁₃, j) —C(O)—NR₁₂R₁₃, k) —Z—SO₂R₁₂, l) —Z—SOR₁₂, m) —Z—SR₁₂, n) —NR₁₂—SO₂R₁₃, o) —NR₁₂—C(O)—R₁₃, p) —NR₁₂—OR₁₃, q) —SO₂—NR₁₂R₁₃, r) —CN, s) —CF₃, t) —C(O) (C₁-C₆)alkyl, u) ═O, v) —Z—SO₂-phenyl or w) —Z—SO₂-het′;

aryl′ is phenyl, naphthyl or biphenyl;

het′ is a 5-, 6- or 7-membered saturated, partially saturated or unsaturated ring containing from one (1) to three (3) heteroatoms independently selected from the group consisting of nitrogen, oxygen and sulfur; and including any bicyclic group in which any of the above heterocyclic rings is fused to a benzene ring or another heterocycle;

provided that:

(1) X—R₁ is other than hydrogen or methyl;

(2) when R₉ and R₁₀ are substituents on the A-ring, they are other than mono- or di-methoxy;

(3) when R₂ and R₃ are taken together to form ═CHR₁, or ═O wherein R₁₁ is —O(C₁-C₆)alkyl, then —X—R₁ is other than (C₁-C₄)alkyl;

(4) when R₂ and R₃ taken together are C═O and R₉ is hydrogen on the A-ring; or when R₂ is hydroxy, R₃ is hydrogen and R₉ is hydrogen on the A-ring, then R₁₀ is other than —O—(C₁-C₆)alkyl or —O—CH₂-phenyl at the 2-position of the A-ring;

(5) when X—R₁ is (C₁-C₄)alkyl, (C₂-C₄)alkenyl or (C₂-C₄)alkynyl, R₉ and R₁O are other than mono-hydroxy or ═O, including the diol form thereof, when taken together; and

(6) when X is absent, R₁ is other than a moiety containing a heteroatom independently selected from N, O or S directly attached to the juncture of the B-ring and the C-ring. (See U.S. Provisional Patent Application No, 60/132,130.).

Each of the glucocorticoid receptor antagonists referenced above and other glucocorticoid receptor antagonists can be used in combination with the compounds of the present invention to treat or prevent diabetes, hyperglycemia, hypercholesterolemia, hypertension, hyperinsulinemia, hyperlipidemia, atherosclerosis, or tissue ischemia.

The compositions of the present invention can also be used in combination with sorbitol dehydrogenase inhibitors. Sorbitol dehydrogenase inhibitors lower fructose levels and have been used to treat or prevent diabetic complications such as neuropathy, retinopathy, nephropathv, cardiomyopathy, microangiopathy, and macroangiopathy. U.S. Pat. Nos. 5,728,704 and 5,866,578 disclose compounds and a method for treating or preventing diabetic complications by inhibiting the enzyme sorbitol dehydrogenase.

Each of the sorbitol dehydrogenase inhibitors referenced above and other sorbitol dehydrogenase inhibitors can be used in combination with the compounds of the present invention to treat diabetes, insulin resistance, diabetic neuropathy, diabetic nephropathy, diabetic retinopathy, cataracts, hyperglycemia, hypercholesterolemia, hypertension, hyperinsulinemia, hyperlipidemia, atherosclerosis, or tissue ischemia.

The compositions of the present invention can also be used in combination with sodium-hydrogen exchanger Type 1 (NHE-1) inhibitors. NHE-1 inhibitors can be used to reduce tissue damage resulting from ischemia. of great concern is tissue damage that occurs as a result of ischemia in cardiac, brain, liver, kidney, lung, gut, skeletal muscle, spleen, pancreas, nerve, spinal cord, retina tissue, the vasculature, or intestinal tissue. NHE-1 inhibitors can also be administered to prevent perioperative myocardial ischemic injury.

Examples of NHE-1 inhibitors include a compound having the Formula Ic

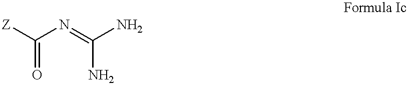

a prodrug thereof or a pharmaceutically acceptable salt of said compound or of said prodrug, wherein the substituents of Formula Ic are as follows:

Z is carbon connected and is a five-membered, diaza, diunsaturated ring having two contiguous nitrogens, said ring optionally mono-, di-, or tri-substituted with up to three substituents independently selected from R¹, R² and R³; or

Z is carbon connected and is a five-membered, triaza, diunsaturated ring, said ring optionally mono- or di-substituted with up to two substituents independently selected from R⁴ and R⁵;

wherein R¹, R², R³, R⁴ and R⁵ are each independently hydrogen, hydroxy(C₁-C₄)alkyl, (C₁-C₄)alkyl, (C₁-C₄)alkylthio, (C₃-C₄)cycloalkyl, (C₃-C₇)cycloalkyl(C₁-C₄)alkyl, (C₁-C₄)alkoxy, (C₁-C₄)alkoxy(C₁-C₄)alkyl, mono-N- or di-N,N-(C₁-C₄)alkylcarbamoyl, M or M(C₁-C₄)alkyl, any of said previous (C₁-C₄)alkyl moieties optionally having from one to nine fluorines; said (C₁-C₄)alkyl or (C₃-C₄) cycloalkyl optionally mono-or di-substituted independently with hydroxy, (C₁-C₄)alkoxy, (C₁-C₄)alkylthio, (C₁-C₄)alkylsulfinyl, (C₁-C₄)alkylsulfonyl, (C₁-C₄)alkyl, mono-N- or di-N,N-(C₁-C₄)alkylcarbamoyl or mono-N- or di-N,N-(C₁-C₄)alkylaminosulfonyl; and said (C₃-C₄)cycloalkyl optionally having from one to seven fluorines;

wherein M is a partially saturated, fully saturated or fully unsaturated five to eight membered ring optionally having one to three heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

said M is optionally substituted, on one ring if the moiety is monocyclic, or one or both rings if the moiety is bicyclic, on carbon or nitrogen with up to three substituents independently selected from R⁶, R⁷ and R⁸, wherein one of R⁶, R⁷ and R⁸ is optionally a partially saturated, fully saturated, or fully unsaturated three to seven membered ring optionally having one to three heteroatoms selected independently from oxygen, sulfur and nitrogen optionally substituted with (C₁-C₄)alkyl and additionally R⁶, R⁷ and R⁸ are optionally hydroxy, nitro, halo, (C₁-C₄)alkoxy, (C₁-C₄)alkoxycarbonyl, (C₁-C₄)alkyl, formyl, (C₁-C₄)alkanoyl, (C₁-C₄)alkanoyloxy, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₄)alkylthio, (C₁-C₄)alkylsulfinyl, (C₁-C₄)alkylsulfonyl, mono-N- or di-N,N-(C₁-C₄)alkylaminosulfonyl, (C₂-C₄)alkenyl, (C₂-C₄)alkynyl or (C₅-C₇) cycloalkenyl,

wherein said (C₁-C₄)alkoxy, (C₁-C₄)alkyl, (C₁-C₇)alkanoyl, (C₁-C₄)alkylthio, mono-N- or di-N,N-(C₁-C₄)alkylamino or (C₃-C₇)cycloalkyl R⁶, R⁷ and R⁸ substituents are optionally mono-substituted independently with hydroxy, (C₁-C₄)alkoxycarbonyl, (C₃-C₇)cycloalkyl, (C₁-C₄)alkanoyl, (C₁-C₄)alkanoylamino, (C₁-C₄) alkanoyloxy, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, nitro, (C₁-C₄)alkylthio, (C₁-C₄)alkylsulfinyl, (C₁-C₄)alkylsulfonyl or mono-N- or di-N,N-(C₁-C₄)alkylaminosulfonyl or optionally substituted with one to nine fluorines. (See PCT patent applciation number PCT/IB99/00206).

Each of the NHE-1 inhibitors referenced above and other NHE-1inhibitors can be used in combination with the compositions of the present invention to treat or prevent diabetes, insulin resistance, diabetic neuropathy, diabetic nephropathy, diabetic retinopathy, cataracts, hyperglycemia, hypercholesterolemia, hypertension, hyperinsulinemia, hyperlipidemia, atherosclerosis, or tissue ischemia.

Other features and embodiments of the invention will become apparent from the following examples which are given for illustration of the invention rather than for limiting its intended scope.

### EXAMPLES

### Example 1

This example discloses preparation of an amorphous solid dispersion of the GPI 5-chloro-1H-indole-2-carboxylic acid [(1S)-benzyl-(2R)-hydroxy-3-((3R, 4S)-dihydroxy-pyrrolidin-1-yl)-3-oxy-propyl]-amide (“Drug 1”), which has a solubility in water of 60 to 80 μg/mL and a solubility in MFD solution of 183 μg/mL. A dispersion of 25 wt % Drug 1 and 75 wt % polymer was made by first mixing Drug 1 in the solvent acetone together with a “medium fine” (AQUOT-MF) grade of the cellulosic enteric polymer HPMCAS (manufactured by Shin Etsu) to form a solution. The solution comprised 1.25 wt % Drug 1, 3.75 wt % HPMCAS, and 95 wt % acetone. This solution was then spray-dried by directing an atomizing spray using a two-fluid external-mix spray nozzle at 2.6 bar (37 psig) at a feed rate of 175 to 180 g/min into the stainless-steel chamber of a Niro XP spray-dryer, maintained at a temperature of 180° C. at the inlet and 69° C. at the outlet.

The resulting amorphous solid spray-dried dispersion (SDD) was collected via a cyclone and then dried in a Gruenberg solvent tray-dryer by spreading the spray-dried particles onto polyethylene-lined trays to a depth of not more than 1 cm and then drying them at 40° C. for at least 8 hours.

### Examples 2-7

Examples 2 through 7 were prepared using the same process as in Example 1, with the exception that different dispersion polymers and different amounts of drug and polymer were used. The variables are noted in Table 1. The SDD of Example 2 was prepared using the Niro PSD-1 spray-dryer. The SDDs of Examples 3-7 were prepared using a “mini” spray dryer, which consisted of an atomizer in the top cap of a vertically oriented stainless steel pipe. The atomizer was a two-fluid nozzle (Spraying Systems Co. 1650 fluid cap and 64 air cap) where the atomizing gas was nitrogen delivered to the nozzle at 100° C. and a flow of 15 gm/min, and the spray-dried solution was delivered to the nozzle at room temperature and a flow rate of 1 gm/min using a syringe pump. Filter paper with a supporting screen was clamped to the bottom end of the pipe to collect the solid spray-dried material and to allow the nitrogen and evaporated solvent to escape.

TABLE 1

| | | | | | Drug Conc. | | | |
|---|---|---|---|---|---|---|---|---|
| | | | | | in the | | | |
| Ex. | | Drug | | Polymer | Disper- | | Solv | Spray |
| No. | Drug | Mass | Polymer* | Mass | sion (wt %) | Solv | Mass | Dryer |
| 1 | 1 | 991 g | HPMCAS-MF | 3009 g | 25 | acetone | 101,670 g | Niro |
| | | | | | | | | XP |
| 2 | 1 | 30 g | HPMCAS-MF | 30 g | 50 | acetone | 2,340 g | Niro |
| | | | | | | | | PSD-1 |
| 3 | 1 | 25 mg | HPMC | 75 mg | 25 | acetone | 10 g | Mini |
| 4 | 1 | 25 mg | PVP | 75 mg | 25 | acetone | 10 g | Mini |
| 5 | 1 | 25 mg | CAP | 75 mg | 25 | acetone | 10 g | Mini |
| 6 | 1 | 25 mg | CAT | 75 mg | 25 | acetone | 10 g | Mini |
| 7 | 1 | 25 mg | HPMCP | 75 mg | 25 | acetone | 10 g | Mini |

*Polymer designations: HPMCAS = hydroxypropyl methyl cellulose acetate succinate, HPMC = hydroxypropyl methyl cellulose, PVP = polyvinylpyrrolidone, CAP = cellulose acetate phthalate, CAT = cellulose acetate trimellitate, HPMCP = hydroxypropyl methyl cellulose phthalate.

### Examples 8-9

Example 8 was prepared by rotoevaporating a polymer:drug solution to dryness. The solution consisted of 7.5 wt % Drug 1, 7.5 wt % HPMCAS-MF, 80.75 wt % acetone, and 4.25 wt % water. The solution was added to a round bottom flask. The flask was rotated at approximately 150 rpm in a 40° C. water bath under a reduced pressure of about 0.1 atm. The resulting solid dispersion was removed from the flask as fine granules and used without further processing.

Example 9 was prepared by spraying a coating solution comprising 2.5 wt % Drug 1, 7.5 wt % HPMCAS-MF, and 90 wt % solvent (5 wt % water in acetone) onto Nu-Core beads (45/60 mesh) to produce a coating of an amorphous solid dispersion of the drug and polymer on the surface of the beads. An analysis showed that the coated beads contained 3.9 wt % Drug 1.

The drug, polymer and solvents for Examples 8 and 9 are shown in Table 2.

TABLE 2

| | | | | | Drug Conc. | | |
|---|---|---|---|---|---|---|---|
| | | | | | in the | | |
| Ex. | | Drug | | Polymer | Dispersion | | Solv |
| No. | Drug | Mass | Polymer | Mass | (wt %) | Solv | Mass |
| 8 | 1 | 1.875 g | HPMCAS-MF | 1.875 g | 50 | 5 wt % | 21.25 g |
| | | | (rotoeveporated) | | | water in | |
| | | | | | | acetone | |
| 9 | 1 | 20 g | HPMCAS-MF | 60 g | 25 | 5 wt % | 720 g |
| | | | (coated beads) | | | water in | |
| | | | | | | acetone | |

### Controls 1-2

Comparative compositions Control 1 and Control 2 were simply 3.6 mg of crystalline Drug 1 and 3.6 mg of the amorphous form of Drug 1 respectively.

### Example 10

In vitro dissolution tests were performed to evaluate the performance of the amorphous dispersions of Examples 1-9 relative to the performance of Controls 1 and 2. The dissolution performance of the SDD of Example 1 was evaluated in an in vitro dissolution test using a microcentrifuge method. In this test, 14.4 mg of the SDD of Example 1 was added to a microcentrifuge tube. The tube was placed in a 37° C. sonicating bath, and 1.8 mL phosphate buffered saline (PBS) at pH 6.5 and 290 mOsm/kg was added. The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and then analyzed by high-performance liquid chromatography (HPLC). The contents of the tubes were mixed on the vortex mixer and allowed to stand undisturbed at 37° C. until the next sample was taken. Samples were collected at 4, 10, 20, 40, 90, and 1200 minutes.

The performance of Example Nos. 2-8 was likewise evaluated in in vitro dissolution tests using the same microcentrifuge method described above. The dosage for each of these tests was 2000 μg/ml. The results of the dissolution tests are shown in Table 3.

The performance of the amorphous dispersions of Example 9 were tested using the same microcentrifuge method, except that 2.5 grams of the coated beads were added to 50 mL of PBS solution (resulting in a dosage of 2000 μg/mL).

For Controls 1 and 2, in vitro dissolution tests were also performed using the same microcentrifuge method except that 3.6 mg of either crystalline or amorphous Drug 1 was used.

TABLE 3

| | | Drug | |
|---|---|---|---|
| | Time | Concentration | AUC |
| Example | (mins) | (μg/mL) | (min* μg/mL) |
| 1 | 0 | 0 | 0 |
| | 4 | 635 | 1,300 |
| | 10 | 644 | 5,100 |
| | 20 | 711 | 11,900 |
| | 40 | 769 | 26,700 |
| | 90 | 844 | 67,000 |
| | 1200 | 1290 | 1,251,400 |
| 2 | 0 | 0 | 0 |
| | 4 | 601 | 1,200 |
| | 10 | 625 | 4,900 |
| | 20 | 653 | 11,300 |
| | 40 | 624 | 24,000 |
| | 90 | 693 | 57,000 |
| | 1200 | 548 | 745,700 |
| 3 | 0 | 0 | 0 |
| | 3 | 544 | 1,100 |
| | 10 | 558 | 4,400 |
| | 20 | 558 | 9,980 |
| | 40 | 552 | 21,100 |
| | 90 | 565 | 49,000 |
| | 1200 | 397 | 582,900 |
| 4 | 0 | 0 | 0 |
| | 3 | 526 | 1,100 |
| | 10 | 637 | 4,500 |
| | 20 | 649 | 11,000 |
| | 40 | 651 | 24,000 |
| | 90 | 688 | 57,400 |
| | 1200 | 409 | 666,300 |
| 5 | 0 | 0 | 0 |
| | 3 | 2066 | 4,100 |
| | 10 | 2035 | 16,400 |
| | 20 | 2075 | 37,000 |
| | 40 | 1965 | 77,400 |
| | 90 | 1845 | 173,600 |
| | 1200 | 255 | 1,338,100 |
| 6 | 0 | 0 | 0 |
| | 3 | 2040 | 4,100 |
| | 10 | 1777 | 15,500 |
| | 20 | 1704 | 32,900 |
| | 40 | 1483 | 64,800 |
| | 90 | 427 | 112,600 |
| | 1200 | 257 | 492,200 |
| 7 | 0 | 0 | 0 |
| | 3 | 1036 | 2,100 |
| | 10 | 1277 | 9,000 |
| | 20 | 1246 | 21,600 |
| | 40 | 1217 | 46,300 |
| | 90 | 503 | 89,300 |
| | 1200 | 350 | 562,700 |
| 8 | 0 | 0 | 0 |
| | 4 | 134 | 270 |
| | 10 | 197 | 1,300 |
| | 20 | 248 | 3,500 |
| | 40 | 308 | 9,100 |
| | 90 | 378 | 26,200 |
| | 1200 | 591 | 564,000 |
| 9 | 0 | 0 | 0 |
| | 4 | 412 | 820 |
| | 10 | 491 | 3,500 |
| | 20 | 523 | 8,600 |
| | 40 | 561 | 19,400 |
| | 90 | 617 | 48,900 |
| | 180 | 752 | 110,500 |
| | 1200 | 967 | 928,000 |
| Control 1 | 0 | 0 | 0 |
| | 4 | 130 | 260 |
| | 10 | 149 | 1,100 |
| | 20 | 139 | 2,500 |
| | 40 | 149 | 5,400 |
| | 90 | 147 | 12,800 |
| | 1200 | 125 | 163,800 |
| Control 2 | 0 | 0 | 0 |
| | 4 | 586 | 1,200 |
| | 10 | 473 | 4,400 |
| | 20 | 220 | 7,800 |
| | 40 | 182 | 11,700 |
| | 90 | 167 | 20,600 |
| | 180 | 158 | 35,200 |
| | 1200 | 203 | 225,900 |

The results of the in vitro dissolution tests are summarized in Table 4, which shows the maximum concentration of Drug 1 in solution during the first 90 minutes of the test (C_{max,90}), the area under the aqueous concentration versus time curve after 90 minutes (AUC₉₀), and the concentration at 1200 minutes (C₁₂₀₀).

TABLE 4

| | | | AUC₉₀ | |
|---|---|---|---|---|
| | Dosage | C_{max, 90} | (min* μg/ | C₁₂₀₀ |
| Example | (μg/mL) | (μg/mL) | mL) | (μg/mL) |
| 1 | 2000 | 844 | 67,000 | 1290 |
| 2 | 2000 | 693 | 57,000 | 548 |
| 3 | 2000 | 565 | 49,000 | 397 |
| 4 | 2000 | 688 | 57,400 | 409 |
| 5 | 2000 | 2075 | 172,600 | 255 |
| 6 | 2000 | 2040 | 112,600 | 257 |
| 7 | 2000 | 1277 | 89,300 | 350 |
| 8 | 2000 | 378 | 26,200 | 591 |
| 9 | 2000 | 617 | 48,900 | 967 |
| Control 1 | 2000 | 149 | 12,800 | 125 |
| Control 2 | 2000 | 586 | 20,600 | 203 |

The results, summarized in Table 4, show that the performance of the SDDs of Examples 1-9 was much better than that of the crystalline drug alone (Control 1), with C_{max,90} values ranging from 2.5- to 13.9-fold that of the crystalline drug, Control 1, and AUC₉₀ values ranging from 2- to 13.4-fold that of the crystalline drug, Control 1. With respect to the amorphous drug alone, the dispersions of Examples 1-9 demonstrated an AUC₉₀ that was 1.27- to 8.4-fold that of the amorphous drug alone, Control 2.

### Example 11

This example shows improved in vivo performance of an amorphous dispersion of Drug 1 and concentration-enhancing polymer compared with the crystalline form of Drug 1. For Example 11, an SDD was prepared following the procedure described in Example 1. The SDD was then formulated as an oral powder for constitution (OPC) by suspending 1.2 gm of the SDD in 100 ml of a 0.5 wt % solution of Polysorbate 80 in sterile water. This OPC, which contained 300 mg of active Drug-1, was taken orally by healthy human subjects (n=4). The dosing bottle was rinsed twice with 100 ml of sterile water and administered orally to the subjects. As a control (Control 3), an OPC was formed using an equivalent quantity of the crystalline form of Drug 1. The results of these in vivo tests are shown in Table 5, giving the maximum concentration of drug achieved in the blood plasma, the time to reach this maximum concentration, and the blood plasma drug AUC from 0 to 24 hours.

TABLE 5

| | Dose | Cₘₐₓ | Time to Cₘₐₓ | AUC₀₋₂₄ |
|---|---|---|---|---|
| Ex. No. | (mg) | (μg/mL) | (hr) | (μg-hr/mL) |
| 11 | 300 | 8.4 ± 1.1 | 2.5 ± 0.6 | 46 ± 7.6 |
| Control 3 | 300 | 1.3 ± 0.3 | 2.3 ± 1.3 | 7.4 ± 3.3 |

As shown in Table 5, the OPC of Example 11 showed improved performance compared with the OPC of Control 3, thus demonstrating the advantage of using an amorphous dispersion of a GPI and concentration-enhancing polymer. Not only was the blood plasma Cₘₐₓ for Example 11 6.5-fold the blood plasma Cₘₐₓ for Control 3, but the blood plasma AUC₀₋₂₄ for Example 11 was 6.21-fold that of Control 3.

### Examples 12-17

These examples demonstrate the utility of the GPI amorphous dispersions of the present invention with another GPI, 5-chloro-1H-indole-2-carboxylic acid [1S-benzyl-2-(3-hydroxy-azetidin-1-yl)-2-oxo-ethyl] amide (“Drug 2”), which has a solubility in water of 14.6 μg/mL. For Example 12, a solution containing 0.5 wt % Drug 2 and 0.5 wt % HPMCAS-LF in acetone was prepared. This solution was pumped into a “mini” spray-dryer apparatus via a syringe pump at a rate of 1.3 mL/min. The polymer solution was atomized through a spray nozzle using a heated stream of nitrogen (100° C.). The resulting solid SDD containing 50 wt % Drug 2 was collected on filter paper at a yield of about 80%.

Examples 13-17 were prepared using the same method used to prepare Example 12, but with different polymers and in some cases different solvents. The variations are noted in Table 6.

TABLE 6

| | | | | | Drug Conc. | | | |
|---|---|---|---|---|---|---|---|---|
| | | | | | in the | | | |
| Ex. | | Drug | | Polymer | Disper- | | Solv | Spray |
| No. | Drug | Mass | Polymer* | Mass | sion (wt %) | Solv | Mass | Dryer |
| 12 | 2 | 25 mg | HPMCAS- | 25 mg | 50 | acetone | 5 g | mini |
| | | | LF | | | | | |
| 13 | 2 | 15 mg | HPMCAS- | 45 mg | 25 | methanol | 10 g | mini |
| | | | LF | | | | | |
| 14 | 2 | 15 mg | HPMCP-55 | 45 mg | 25 | methanol | 10 g | mini |
| 15 | 2 | 15 mg | PVP | 45 mg | 25 | 10 wt % water | 11 g | mini |
| | | | | | | in methanol | | |
| 16 | 2 | 150 mg | CAP | 150 mg | 50 | 50 wt % water | 33.4 g | mini |
| | | | | | | in acetone | | |
| 17 | 2 | 150 mg | CAT | 150 mg | 50 | 50 wt % water | 33.4 g | mini |
| | | | | | | in acetone | | |

*Polymer designations: HPMCAS = hydroxypropyl methyl cellulose acetate succinate, PVP = polyvinylpyrrolidone, CAP = cellulose acetate phthalate, CAT = cellulose acetate trimellitate, HPMCP = hydroxypropyl methyl cellulose phthalate.

### Controls 4-5

Comparative compositions Control 4 and Control 5 were simply 1.8 mg of crystalline Drug 2 and 1.8 mg of amorphous Drug 2, respectively.

### Example 18

In vitro dissolution tests were performed to evaluate the performance of the amorphous dispersions of Examples 12-17 relative to the performance of Controls 4 and 5. The SDD of Example 12 was evaluated in an in vitro dissolution test using a microcentrifuge method. In this test, 3600 μg of the SDD of Example 12 was added to a microcentrifuge tube. The tube was placed in a 37° C. sonicating bath, and 1.8 mL of model fasted duodenal solution (MFDS), comprising phosphate buffered saline with 14.7 mM sodium taurocholic acid and 2.8 mM of 1-palmitoyl-2-oleyl-sn-glycero-3-phosphocholine, pH 6.5, 290 mOsm/kg, was added. This resulted in a dose of Drug 2 of 1000 μg/ml. The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and then analyzed by high-performance liquid chromatography (HPLC). The contents of the tubes were mixed on the vortex mixer and allowed to stand undisturbed at 37° C. until the next sample was taken. Samples were collected at 4, 10, 20, 40, 90, and 1200 minutes.

For Controls 4 and 5, in vitro dissolution tests were performed using the procedures described above except that 1.8 mg of crystalline and amorphous Drug 2 was used, respectively.

Results of the dissolution tests are presented in Table 7.

TABLE 7

| | | Drug | |
|---|---|---|---|
| | Time | Concentration | AUC |
| Example | (mins) | (μg/mL) | (min* μg/mL) |
| 12 | 0 | 0 | 0 |
| | 4 | 850 | 1,700 |
| | 10 | 866 | 6,900 |
| | 20 | 895 | 15,700 |
| | 40 | 908 | 33,700 |
| | 90 | 923 | 79,500 |
| | 1200 | 933 | 1,109,500 |
| 13 | 0 | 0 | 0 |
| | 4 | 958 | 1,900 |
| | 10 | 993 | 7,800 |
| | 20 | 997 | 17,700 |
| | 40 | 975 | 37,400 |
| | 90 | 992 | 86,600 |
| | 1200 | 961 | 1,170,500 |
| 14 | 0 | 0 | 0 |
| | 4 | 860 | 1,700 |
| | 10 | 822 | 6,800 |
| | 20 | 804 | 14,900 |
| | 40 | 805 | 31,000 |
| | 90 | 778 | 70,600 |
| | 1200 | 732 | 908,600 |
| 15 | 0 | 0 | 0 |
| | 4 | 467 | 930 |
| | 10 | 498 | 3,800 |
| | 20 | 505 | 8,800 |
| | 40 | 507 | 19,000 |
| | 90 | 495 | 44,000 |
| | 1200 | 545 | 621,200 |
| 16 | 0 | 0 | 0 |
| | 4 | 696 | 1,400 |
| | 10 | 708 | 5,600 |
| | 20 | 708 | 12,700 |
| | 40 | 695 | 26,700 |
| | 90 | 701 | 61,600 |
| | 1200 | 735 | 858,600 |
| 17 | 0 | 0 | 0 |
| | 4 | 768 | 1,500 |
| | 10 | 766 | 6,100 |
| | 20 | 746 | 13,700 |
| | 40 | 730 | 28,500 |
| | 90 | 744 | 65,300 |
| | 1200 | 722 | 878,900 |
| Control 4 | 0 | 0 | 0 |
| | 4 | 45 | 90 |
| | 10 | 44 | 357 |
| | 20 | 53 | 842 |
| | 40 | 72 | 2,100 |
| | 90 | 82 | 5,900 |
| | 1200 | 102 | 108,100 |
| Control 5 | 0 | 0 | 0 |
| | 4 | 151 | 302 |
| | 10 | 203 | 1,400 |
| | 20 | 225 | 3,500 |
| | 40 | 238 | 8,100 |
| | 90 | 268 | 20,800 |
| | 1200 | 370 | 374,900 |

The results of these tests are summarized in Table 8, which shows the maximum concentration of Drug 2 in solution during the first 90 minutes of the test (C_{max,90}), the aqueous area under the curve after 90 minutes (AUC₉₀), and the concentration at 1200 minutes (C₁₂₀₀).

TABLE 8

| | | | AUC₉₀ | |
|---|---|---|---|---|
| | Dosage | C_{max, 90} | (min* μg/ | C₁₂₀₀ |
| Example | (μg/mL) | (μg/mL) | mL) | (μg/mL) |
| 12 | 1000 | 923 | 79,500 | 933 |
| 13 | 1000 | 997 | 86,600 | 961 |
| 14 | 1000 | 860 | 70,600 | 732 |
| 15 | 1000 | 507 | 44,000 | 545 |
| 16 | 1000 | 708 | 61,600 | 735 |
| 17 | 1000 | 768 | 65,300 | 722 |
| Control 4 | 1000 | 82 | 5,900 | 102 |
| Control 5 | 1000 | 268 | 20,800 | 370 |

In general, the dispersions of Examples 12-17 showed much better performance than the crystalline drug alone, with C_{max,90} values ranging from 6.2- to 12.1-fold that of the crystalline drug, Control 4, and AUC₉₀ values ranging from 7.5- to 14.7-fold that of the crystalline drug, Control 4. With respect to the amorphous drug alone, all of the dispersions of Examples 12-17 demonstrated a Cₘₐₓ and an AUC₉₀ greater than that of the amorphous drug alone, with C_{max,90} values ranging from 1.9-to 3.7-fold that of the amorphous drug, Control 5, and AUC₉₀ values ranging from 2.1- to 4.2-fold that of the amorphous drug, Control 5.

### Example 19

This example demonstrates that the compositions of this invention, when orally dosed to beagle dogs, give a high systemic compound exposure (Cₘₐₓ and AUC). An amorphous solid dispersion of 50 wt % Drug 2 and 50 wt % polymer was made by first mixing Drug 2 in the solvent acetone together with HPMCAS-LF to form a solution. The solution comprised 2.5 wt % Drug 2, 2.5 wt % HPMCAS-LF, and 95 wt % acetone. This solution was then spray-dried by directing an atomizing spray using a two-fluid external-mix spray nozzle at 2.2 bar at a feed rate of 200 g/min into the stainless-steel chamber of a Niro PSD-1 spray-dryer, maintained at a temperature of 180° C. at the inlet and 68° C. at the outlet.

The resulting amorphous solid SDD was collected via a cyclone and then dried in a Gruenberg solvent tray-dryer by spreading the spray-dried particles onto polyethylene-lined trays to a depth of not more than 1 cm and then drying them at 40° C. for at least 8 hours.

The SDD was dosed as an oral powder for constitution (OPC) by suspending 200 mg of the SDD in approximately 20 ml of a 2 wt % solution of Polysorbate 80 in sterile water. This OPC, containing 100 mg of active Drug 2 was administered orally to beagle dogs using an oral gavage tube. As a control (Control 6), a similar OPC was formed using the crystalline form of the drug. Relative bioavailability was calculated by dividing the AUC in the blood of subjects receiving the test dose by the AUC in the blood of subjects receiving the control dose (Control 6).

Dogs that had fasted overnight were dosed with suspensions containing 100 mg of Drug 2, along with 20 mL of water. Blood was collected from the jugular vein of the dogs before dosing and at various time points after dosing. To 100 μL of each plasma sample, 5 mL of methyl-tert-butyl ether (MTBE) and 1 mL of 500 mM sodium carbonate buffer (pH 9) were added; the sample was vortexed for 1 minute and then centrifuged for 5 minutes. The aqueous portion of the sample was frozen in a dry-ice/acetone bath, and the MTBE layer was decanted and evaporated in a vortex evaporator. Dried samples were reconstituted in 100 μL of mobile phase (33% acetonitrile and 67% of 0.1% formic acid in water). Analysis was carried out by HPLC. The results of these tests are shown in Table 9, where Cₘₐₓ is the maximum concentration in the blood plasma, AUC₀₋₂₄ is the area under the drug concentration in the blood curve in the first 24 hours, and Relative Bioavailability is the AUC in the blood of subjects receiving the test dose divided by the AUC of subjects receiving the Control 6.

TABLE 9

| Example | C_{max,} | AUC₀₋₂₄ | Relative |
|---|---|---|---|
| No. | (μg/mL) | (μg-hr/mL) | Bioavailability |
| 19 | 9.8 ± 4.6 | 38 ± 6 | 6.2 |
| Control 6 | 1.6 ± 0.7 | 6.1 ± 4.0 | 1 |

The results show the superior performance of the amorphous GPI and polymer dispersion of Example 19 relative to the crystalline GPI, Control 6, providing a Cₘₐₓ value that was 6.1-fold that of the control and a relative bioavailability of 6.2 relative to the control.

### Examples 20-25

Examples 20-25 demonstrate the utility of the GPI amorphous dispersions of the present invention with another GPI, 5-chloro-1H-indole-2-carboxylic acid [(1S)-((R)-hydroxy-methoxy-methylcarbamoylmethyl)-2-phenyl-ethyl]-amide (“Drug 3”), which has a solubility in water of 1 μg/mL and a solubility in MFD solution of 17 μg/mL. To prepare Example 20, a solution containing 0.5 wt % of Drug 3 and 0.5 wt % HPMCAS-MF in acetone was prepared. This solution was pumped into a “mini” spray-dryer apparatus via a syringe pump at a rate of 1.3 mL/min. The polymer solution was atomized through a spray nozzle using a heated stream of nitrogen (100° C.). The resulting solid SDD containing 50 wt % Drug 3 was collected on a filter paper at a yield of about 62%.

Examples 21-25 were prepared using the same method used to prepare Example 20, but with different polymers and in some cases different solvents. The variations are note in Table 10.

TABLE 10

| | | | | | Drug Conc. | | | |
|---|---|---|---|---|---|---|---|---|
| | | | | | in the | | | |
| Ex. | | Drug | | Polymer | Disper- | | Solv | Spray |
| No. | Drug | Mass | Polymer* | Mass | sion (wt %) | Solv | Mass | Dryer |
| 20 | 3 | 52 mg | HPMCAS-MF | 52 mg | 50.0 | acetone | 12 g | mini |
| 21 | 3 | 50.5 mg | PVP | 50.4 mg | 50.0 | acetone | 12 g | mini |
| | | | | | | methanol | 0.24 g | |
| 22 | 3 | 49.7 mg | HPMCP | 49.9 mg | 49.9 | acetone | 12 g | mini |
| 23 | 3 | 50.1 mg | CAP | 50.3 mg | 49.9 | acetone | 12 g | mini |
| 24 | 3 | 50.9 mg | HPC | 51.8 mg | 49.6 | acetone | 12 g | mini |
| 25 | 3 | 50 mg | PVAP | 50 mg | 50.0 | acetone | 12 g | mini |

### Control 8

Comparative composition Control 8 consisted of 5 mg of the crystalline form of Drug 3 alone.

### Example 26

In vitro dissolution tests were performed to evaluate the performance of the amorphous dispersions of Examples 20-25 relative to the performance of Control 8. The SDD of Example 20 was evaluated in an in vitro dissolution test using a syringe/filter method. In this test, 10 mg of the SDD of Example 20 was added to 10 mL of MFD solution, comprising phosphate buffered saline with 14.7 mM sodium taurocholic acid and 2.8 mM of 1-palmitoyl-2-oleyl-sn-glycero-3-phosphocholine, pH 6.5, 290 mOsm/kg. The drug solution was added to a 10 mL polypropylene syringe fitted with a Titan PVDF 0.45 μm filter. The syringe was attached to a vertical rotating wheel in a 37° C. constant temperature chamber. At each sampling time, 13 drops were expelled from the syringe through the filter. The filtrate was then diluted 1:1 (by volume) with methanol and analyzed by high-performance liquid chromatography (HPLC). Between sampling times, the test solution was mixed as the syringe was rotated on the wheel at 37° C. Samples were collected at 0.5, 5, 30, 60, 180, and 1200 minutes.

In vitro dissolution tests for Examples 21-25 were performed using the same procedure described above for Example 20.

For Control 8, an in vitro dissolution test was performed using the procedure described above except that 5 mg of crystalline Drug 3 was used.

The concentrations of drug obtained in these samples are shown in Table 11 below.

TABLE 11

| | | Drug | |
|---|---|---|---|
| | Time | Concentration | AUC |
| Example | (mins) | (μg/mL) | (min* μg/mL) |
| 20 | 0 | 0 | 0 |
| | 0.5 | 25 | 6 |
| | 5 | 62 | 202 |
| | 30 | 112 | 2,400 |
| | 60 | 115 | 5,800 |
| | 180 | 120 | 19,900 |
| | 1200 | 14 | 88,200 |
| 21 | 0 | 0 | 0 |
| | 0.5 | 8 | 2 |
| | 5 | 33 | 94 |
| | 30 | 121 | 2,000 |
| | 60 | 128 | 5,800 |
| | 180 | 114 | 20,300 |
| | 1200 | 13 | 85,000 |
| 22 | 0 | 0 | 0 |
| | 0.5 | 12 | 3 |
| | 5 | 48 | 138 |
| | 30 | 112 | 2,100 |
| | 60 | 128 | 5,700 |
| | 180 | 106 | 19,800 |
| | 1200 | 13 | 80,500 |
| 23 | 0 | 0 | 0 |
| | 0.5 | 14 | 4 |
| | 5 | 46 | 139 |
| | 30 | 106 | 2,000 |
| | 60 | 121 | 5,400 |
| | 180 | 127 | 20,300 |
| | 1200 | 13 | 91,700 |
| 24 | 0 | 0 | 0 |
| | 0.5 | 24 | 6 |
| | 5 | 62 | 200 |
| | 30 | 94 | 2,200 |
| | 60 | 95 | 5,000 |
| | 180 | 91 | 16,100 |
| | 1200 | 11 | 68,200 |
| 25 | 0 | 0 | 0 |
| | 0.5 | 6 | 2 |
| | 5 | 34 | 92 |
| | 30 | 89 | 1,600 |
| | 60 | 104 | 4,500 |
| | 180 | 17 | 11,800 |
| | 1200 | 9 | 25,000 |
| Control 8 | 0 | 0 | 0 |
| | 0.5 | 3 | 1 |
| | 5 | 8 | 26 |
| | 30 | 10 | 251 |
| | 60 | 10 | 551 |
| | 180 | 9 | 1,700 |
| | 1200 | 8 | 10,400 |

The results of these test are summarized in Table 12, which shows the maximum concentration of Drug 3 in solution after 180 minutes (Cₘₐₓ₁₈₀), the aqueous area under the curve after 180 minutes (AUC₁₈₀), and the concentration at 1200 minutes (C₁₂₀₀)

TABLE 12

| | | | AUC₁₈₀ | |
|---|---|---|---|---|
| | Dosage | Cₘₐₓ₁₈₀ | (min* μg/ | C₁₂₀₀ |
| Example | (μg/mL) | (μg/mL) | mL) | (μg/mL) |
| 20 | 500 | 120 | 19,900 | 14 |
| 21 | 500 | 133 | 20,300 | 13 |
| 22 | 500 | 127 | 19,800 | 13 |
| 23 | 500 | 127 | 20,300 | 13 |
| 24 | 500 | 97 | 16,100 | 11 |
| 25 | 500 | 104 | 11,800 | 9 |
| Control 8 | 500 | 10 | 1,700 | 8 |

The results show that the performance of the SDD of Examples 20-25 was much better than that of the crystalline drug alone, with Cₘₐₓ₁₈₀ values 9.7- to 13.3-fold that of Control 8, and AUC₁₈₀ values 6.9- to 11.9-fold that of Control 8.

### Examples 27-29

These examples disclose simple physical mixtures of a GPI and a concentration-enhancing polymer. Mixtures of Drug 1 and HPMCAS-MF were formed by dry mixing amorphous Drug 1 with HPMCAS-MF. For Example 27, the composition comprised 3.6 mg (75 wt %) Drug 1 and 1.2 mg (25 wt %) HPMCAS-MF; for Example 28, the composition comprised 3.6 mg (50 wt %) Drug 1 and 3.6 mg (50 wt %) HPMCAS-MF; for Example 29, the composition comprised 3.6 mg (25 wt %) Drug 1 and 10.8 mg (75 wt %) HPMCAS-MF.

These compositions were evaluated in in vitro dissolution tests using the procedures outlined in Example 10. The quantities of drug and polymer noted above were each added to a microcentrifuge tube, to which was added 1.8 ml of PBS solution. The tube was vortexed immediately after adding the PBS solution. The results of these dissolution tests are given in Table 13, and summarized in Table 14.

TABLE 13

| | | Drug 1 | |
|---|---|---|---|
| | Time | Concentration | AUC |
| Example | (mins) | (μg/mL) | (min* μg/mL) |
| 27 | 0 | 0 | 0 |
| 75 wt % | 4 | 714 | 1,400 |
| Drug 1/ | 10 | 737 | 5,800 |
| 25 wt % | 20 | 696 | 12,900 |
| HPMCAS-MF | 40 | 690 | 26,800 |
| | 90 | 729 | 62,300 |
| | 180 | 684 | 125,800 |
| | 1200 | 440 | 696,600 |
| 28 | 0 | 0 | 0 |
| 50 wt % | 4 | 377 | 755 |
| Drug 1/ | 10 | 370 | 3,000 |
| 50 wt % | 20 | 836 | 9,000 |
| HPMCAS-MF | 40 | 846 | 25,800 |
| | 90 | 898 | 69,500 |
| | 180 | 918 | 151,200 |
| | 1200 | 627 | 932,700 |
| 29 | 0 | 0 | 0 |
| 25 wt % | 4 | 999 | 2,000 |
| Drug 1/ | 10 | 1030 | 8,100 |
| 75 wt % | 20 | 1065 | 18,600 |
| HPMCAS-MF | 40 | 1133 | 40,600 |
| | 90 | 1185 | 98,500 |
| | 180 | 1304 | 210,500 |
| | 1200 | 1379 | 1,579,500 |

TABLE 14

| | | | AUC₉₀ | |
|---|---|---|---|---|
| | Dosage | C_{max, 90} | (min* μg/ | C₁₂₀₀ |
| Example | (μg/mL) | (μg/mL) | mL) | (μg/mL) |
| 27 | 2000 | 729 | 62,300 | 440 |
| 28 | 2000 | 898 | 69,500 | 627 |
| 29 | 2000 | 1185 | 98,500 | 1379 |
| Control 2 | 2000 | 586 | 20,600 | 203 |

These simple physical mixtures of amorphous Drug 1 and HPMCAS-MF showed much better performance than the amorphous drug alone (Control 2, shown in Table 14 for comparison), with C_{max,90} values that were 1.24- to 2.0-fold that of Control 2, and AUC₉₀ values that were 3.0- to 4.8-fold that of Control 2.

### Example 30

This example demonstrates another simple physical mixture of amorphous GPI and polymer. A coating solution comprising 7.5 wt % HPMCAS-MF dissolved in 92.5 wt % solvent (5 wt % water in acetone) was prepared and spray-coated onto Nu-Core Beads (45/60 mesh), producing a thin coating of the polymer on the surface of the beads resulting in beads containing 12.2 wt % HPMCAS-MF. Samples of these beads (2.4 gm) were then mixed with 100 mg of amorphous Drug 1 (resulting in a drug:polymer ratio of 1:3 or 25 wt % Drug 1) and evaluated in an in vitro dissolution test using the procedures outlined in Example 10. The results of the dissolution test are presented in Table 15.

TABLE 15

| | | Drug 1 | |
|---|---|---|---|
| | Time | Concentration | AUC |
| Example | (mins) | (μgA/mL) | (min* μg/mL) |
| 30 | 0 | 0 | 0 |
| | 4 | 797 | 1,600 |
| | 10 | 1047 | 7,100 |
| | 20 | 1292 | 18,800 |
| | 40 | 1523 | 47,000 |
| | 90 | 1653 | 126,400 |
| | 180 | 1724 | 278,300 |
| | 1200 | 1885 | 2,113,600 |

The physical mixture of HPMCAS-MF coated beads with amorphous Drug 1 showed improved performance over crystalline Drug 1 alone, with a C_{max,90} value that is 11-fold that of crystalline Drug 1 (Control 1) and an AUC₉₀ value that is 9.9-fold that of Control 1.

### Example 31

A composition was formed by blending 50 wt % of the SDD of Example 2 (containing 50 wt % Drug 1 and 50 wt % HPMCAS-MF) with 50 wt % HPMCAS-MF. This composition was evaluated in a dissolution test as described in Example 10. The results of this test are presented in Table 16, and show that the blend of the SDD with polymer performs well, with a C_{max,90} value that is 6.6-fold that of the crystalline drug alone (Control 1) and an AUC₉₀ value that is 6.2-fold that of Control 1.

TABLE 16

| | | Drug 1 | |
|---|---|---|---|
| | Time | Concentration | AUC |
| Example | (mins) | (μg/mL) | (min* μg/mL) |
| 31 | 0 | 0 | 0 |
| | 4 | 766 | 1,500 |
| | 10 | 840 | 6,400 |
| | 20 | 874 | 14,900 |
| | 40 | 884 | 32,500 |
| | 90 | 979 | 79,100 |
| | 1200 | 1133 | 1,251,000 |

### Examples 32-35

An amorphous solid dispersion of 50 wt % Drug 1 and 50 wt % polymer was made by first mixing Drug 1 in a solvent together with HPMCAS-MF to form a solution. The solution comprised 7.5 wt % Drug 1, 7.5 wt % HPMCAS, 80.75 wt % acetone and 4.25 wt % water. This solution was then spray-dried by directing an atomizing spray using a two-fluid external-mix spray nozzle at 2.7 bar (37 psig) at a feed rate of 175 g/min into the stainless-steel chamber of a Niro spray-dryer, maintained at a temperature of 175° C. at the inlet and 70° C. at the outlet.

The resulting amorphous solid spray-dried dispersion (SDD) was collected via a cyclone and then dried in a Gruenberg solvent tray-dryer by spreading the spray-dried particles onto polyethylene-lined trays to a depth of not more than 1 cm and then drying them at 40° C. for 16 hours.

The SDD above was incorporated into tablets containing 25, 50, 100, and 200 mg. Tablets with a dose of 25 mg (Example 32) consisted of 7.14 wt % SDD, 40.0 wt % HPMCAS-MF, 49.11 wt % microcrystalline cellulose (Avicel® PH 102), 3.0 wt % croscarmellose sodium (Ac-Di-Sol®) and 0.75 wt % magnesium stearate. Tablets with a dose of 50 mg (Example 33) consisted of 14.29 wt % SDD, 40.0 wt % HPMCAS-MF, 41.96 wt % Avicel® PH 102, 3.0 wt % Ac-Di-Sol®, and 0.75 wt % magnesium stearate. Tablets with a dose of 100 mg (Example 34) consisted of 28.57 wt % SDD, 30.0 wt % HPMCAS-MF, 37.68 wt % Avicel® PH 102, 3.0 wt % Ac-Di-Sol®, and 0.75 wt % magnesium stearate. Tablets with a dose of 200 mg (Example 35) consisted of 57.14 wt % SDD, 39.11 wt % Avicel® PH 102, 3.0 wt % Ac-Di-Sol®, and 0.75 wt % magnesium stearate. In each case, the targeted tablet weight was 700 mg.

To form the tablets, the SDD was first granulated (roller compacted) on a Freund TF-mini roller compactor using an auger speed of 30 rpm, a roller speed of 4 rpm, and a roller pressure of 30 Kg_{f}/cm². The resulting compacted material was then reduced using a mini-Comil at a power setting of 4, with sieve 039R. The milled SDD was then blended in a V-blender with the HPMCAS-MF, Avicel®, and Ac-Di-Sol® for 20 minutes using the proportions noted above. Next, a portion of the magnesium stearate (about 20 wt % of the total magnesium stearate used) was added and the material was blended for 5 minutes. The blend was then granulated again using an auger speed of 20 rpm, a roller speed of 4 rpm, and a roller pressure of 30 Kg_{f}/cm². The resulting compacted material was then reduced using a Comill with a power setting of 3 and a sieve size of 032R. The remaining magnesium stearate was then added, and the material was blended for 5 minutes in a V-blender. This material was then formed into tablets using 0.3437×0.6875-inch oval tooling on a Kilian T-100 tablet press with precompression of 1 to 2 kN and a compression force of 10 kN.

To test in vitro drug dissolution, one of each of the tablets was each placed in 200 mL of a gastric buffer solution (0.1 N HCl at pH 1.2) for 30 minutes at 37° C. and stirred, after which 50 mL of a pH 13 buffer solution was added to produce a final pH of 7.5 and a final volume of 250 mL. The drug concentration was determined over time by periodically withdrawing samples, centrifuging the samples to remove any undissolved drug, diluting the supernatant in methanol, analyzing the samples by HPLC, and calculating drug concentrations. The concentration of drug obtained in in vitro dissolution tests are shown in Table 17 below.

TABLE 17

| | | Drug 1 | |
|---|---|---|---|
| | Time | Concentration | AUC |
| Example | (mins) | (μg/mL) | (min* μg/mL) |
| 32 | 0 | 0 | 0 |
| 25 mg | 5 | 6 | 16 |
| | 15 | 13 | 110 |
| | 20 | 15 | 178 |
| | 35 | 25 | 478 |
| | 45 | 30 | 755 |
| | 60 | 36 | 1,300 |
| | 75 | 43 | 1,800 |
| | 90 | 50 | 2,500 |
| | 120 | 58 | 4,200 |
| | 180 | 65 | 7,900 |
| | 1200 | 96 | 90,200 |
| 33 | 0 | 0 | 0 |
| 50 mg | 5 | 9 | 24 |
| | 15 | 19 | 166 |
| | 20 | 23 | 271 |
| | 35 | 42 | 755 |
| | 45 | 61 | 1,300 |
| | 60 | 82 | 2,300 |
| | 75 | 99 | 3,700 |
| | 90 | 111 | 5,300 |
| | 120 | 130 | 8,900 |
| | 180 | 152 | 17,400 |
| | 1200 | 202 | 197,800 |
| 34 | 0 | 0 | 0 |
| 100 mg | 5 | 20 | 49 |
| | 15 | 43 | 361 |
| | 20 | 50 | 594 |
| | 35 | 112 | 1,800 |
| | 45 | 150 | 3,100 |
| | 60 | 186 | 5,700 |
| | 75 | 199 | 8,500 |
| | 90 | 213 | 11,600 |
| | 120 | 236 | 18,300 |
| | 180 | 260 | 33,200 |
| | 1200 | 381 | 360,300 |
| 35 | 0 | 0 | 0 |
| 200 mg | 5 | 26 | 64 |
| | 15 | 64 | 514 |
| | 20 | 81 | 878 |
| | 35 | 168 | 2,800 |
| | 45 | 424 | 5,700 |
| | 60 | 470 | 12,400 |
| | 75 | 479 | 19,500 |
| | 90 | 502 | 26,900 |
| | 120 | 518 | 42,200 |
| | 180 | 522 | 73,400 |
| | 1200 | 298 | 491,000 |

The data demonstrate that approximately all of the drug had been released by 1200 minutes.

### Example 36

An amorphous solid dispersion of 67 wt % Drug 3 and 33 wt % polymer was made by first mixing Drug 3 in the solvent acetone together with HPMCAS-MF to form a solution. The solution comprised 3.33 wt % Drug 3, 1.67 wt % HPMCAS-MF, and 95 wt % acetone. This solution was then spray-dried by directing an atomizing spray using a two-fluid external-mix spray nozzle at 0.6 bar at a feed rate of 75 g/min into the stainless-steel chamber of a Niro PSD-1 spray-dryer, maintained at a temperature of 120° C. at the inlet and 76° C. at the outlet.

The resulting amorphous solid spray-dried dispersion (SDD) was collected via a cyclone and then dried in a Gruenberg solvent tray-dryer by spreading the spray-dried particles onto polyethylene-lined trays to a depth of not more than 1 cm and then drying them at 40° C. for at least 8 hours.

### Example 37

Capsules containing a total mass of 500 mg were prepared using the SDD of Drug 3 from Example 36. Each capsule contained 60 wt % of the SDD, 15 wt % Fast Flo lactose, 15 wt % Avicel PH-102, 7 wt % Explotab, 2 wt % sodium lauryl sulfate, and 1 wt % magnesium stearate, resulting in capsules containing 200 mg of Drug 3.

### Example 38

Tablets with a total mass of 600 mg were prepared containing 50 wt % SDD from Example 36, 32 wt % Avicel PH-102, 11 wt % Fast Flo lactose, 5 wt % Explotab, 1 wt % sodium lauryl sulfate, and 1 wt % magnesium stearate, resulting in tablets containing 200 mg of Drug 3.

### Examples 39-40

Capsules with a total mass of 600 mg were prepared, each capsule containing 50 wt % SDD from Example 36, 32 wt % Avicel PH-102, 11 wt % Fast Flo lactose, 5 wt % Explotab, 1 wt % sodium lauryl sulfate, and 1 wt % magnesium stearate (Example 39), resulting in capsules containing 200 mg of Drug 3. Example 40 was prepared by coating the capsules of Example 38 with cellulose acetate phthalate.

### Example 41

The dosage forms of Examples 37 to 40 were tested in in vivo tests. Beagle dogs that had fasted overnight were dosed with capsules and tablets from Examples 37 to 40, along with 50 mL of water. Blood was collected from the jugular vein of the dogs before dosing and at various time points after dosing. To 100 μL of each plasma sample, 5 mL of methyl-tert-butyl ether (MTBE) and 1 mL of 500 mM sodium carbonate buffer (pH 9) were added; the sample was vortexed for 1 minute and then centrifuged for 5 minutes. The aqueous portion of the sample was frozen in a dry-ice/acetone bath, and the MTBE layer was decanted and evaporated in a vortex evaporator. Dried samples were reconstituted in 100 μL of mobile phase (33% acetonitrile and 67% of 0.1% formic acid in water). Analysis was carried out by HPLC.

As a control (Control 9), an OPC was formed using the crystalline form of Drug 3 as follows. An aqueous suspension of 200 mg of crystalline drug was prepared in 2 wt % Polysorbate 80 in water. Oral administration of the aqueous drug suspensions was facilitated using an oral gavage equipped with a polyethylene tube insert. The polyethylene tube insert was used to accurately deliver the desired volume of dose by displacement, without the need for additional volume of water to rinse the tube.

The results of these tests are shown in Table 18, where Cₘₐₓ is the maximum concentration of Drug 3 in the blood plasma, AUC₀₋₂₄ is the area under the curve in the first 24 hours, and Relative Bioavailability is the AUC in the blood of the test dose divided by the AUC in the blood of the reference dose (Control 9). The results show that the relative bioavailabilities obtained with the dosage forms of the present invention are 2.8 to 6.2 relative to Control 9. Furthermore, the Cₘₐₓ of the dosage forms of the present invention were 2.6-fold to 4.7-fold that of Control 9.

TABLE 18

| | | | | | Relative |
|---|---|---|---|---|---|
| Exam- | Formu- | Dose | C_{max,} | AUC₀₋₂₄ | Bioavail- |
| ple | lation | (mg) | (μg/mL) | (μg-hr/mL) | ability |
| Control | Crystal- | 200 | 1.03 ± | 6.48 ± | — |
| 9 | line suspen- | | 0.57 | 3.60 | |
| | sion | | | | |
| 37 | Capsule | 200 | 3.81 ± | 31.75 ± | 6.0 |
| | | | 2.39 | 18.61 | |
| 38 | Tablet | 200 | 2.84 ± | 26.09 ± | 4.2 |
| | | | 2.04 | 21.43 | |
| 39 | Capsule | 200 | 4.86 ± | 40.55 ± | 6.2 |
| | | | 2.30 | 20.74 | |
| 40 | CAP coated | 200 | 2.67 ± | 18.08 ± | 2.8 |
| | Capsule | | 2.45 | 12.21 | |

### Example 42

This example illustrates a method for making a tablet dosage form of the present invention containing an amorphous dispersion of Drug 1. An amorphous solid dispersion of Drug 1 and HPMCAS was made by mixing Drug 1 in a solvent together with HPMCAS to form a solution, and then spray-drying the solution. The solution comprised 7.5 wt % Drug 1, 7.5 wt % HPMCAS-MF, 4.25 wt % water, and 80.75 wt % acetone. The solution was then spray-dried by directing an atomizing spray using a two-fluid external-mix spray nozzle at 2.7 bar at a feed rate of 175 g/min into the stainless steel chamber of a Niro spray-dryer, maintained at a temperature of 140° C. at the inlet and 50° C. at the outlet. The resulting SDD was collected via a cyclone and then dried in a Gruenberg solvent tray-dryer by spreading the spray-dried particles onto polyethylene-lined trays to a depth of not more than 1 cm and then drying them at 40° C. for at least 8 hours. After drying, the SDD contained 50 wt % Drug 1.

The tablets contained 50 wt % SDD, 25 wt % anhydrous dibasic calcium phosphate, 12 wt % Avicel® PH 200, 12.5 wt % crospovidone, and 0.5 wt % magnesium stearate. The total batch weight was 190 g. First, the ingredients, except for magnesium stearate, were added to a Turbula blender and blended for 20 minutes. Next, half of the magnesium stearate was added and blended for 5 minutes. The blend was then roller-compacted with a Vector TF mini roller compactor using an auger speed of 30 rpm, a roller speed of 5 rpm, and a roller pressure of 35.2 Kgf/cm². The resulting compacted material was then milled using a Quadro Comil 193AS mill at a power setting of 3, using impeller 2B-1607-005 and Screen 2B-075R03151173. The second half of the magnesium stearate was added next, and the material was blended for 5 minutes in a Turbula blender. This material was then formed into 800 mg tablets using 1/2-inch SRC tooling on a Manesty F press. An average tablet hardness of 19 Kp was obtained. Average disintegration time in deionized water (USP disintegration apparatus) was 2 minutes, 50 seconds.

### Example 43

The tablets of Example 42 were coated in a LDCS 20 pan-coater using an 8 wt % aqueous solution of Opadry® II Clear. The following coating conditions were used: tablet bed weight, 900 g; pan speed, 20 rpm; outlet temperature, 40° C.; solution flow, 8 g/min; atomization pressure, 20 psi; and air flow, 40 cfm. The coating weight gain was 3 wt %. The resulting average coated tablet hardness was 45 Kp. Average disintegration time in deionized water was 4 minutes, 57 seconds.

### Example 44

This example illustrates another method for making a tablet dosage form of the present invention containing an amorphous dispersion of Drug 1. An amorphous solid dispersion of Drug 1 and HPMCAS was made by mixing Drug 1 in a solvent together with HPMCAS to form a solution, and then spray-drying the solution, as described in Example 42. The tablets contained 50 wt % of the SDD, 25 wt % anhydrous dibasic calcium phosphate, 12 wt % Avicel® PH 105 QS, 12.5 wt % crospovidone, and 0.5 wt % magnesium stearate. To form the tablets, the ingredients, except magnesium stearate, were first added to a V-blender and blended for 20 minutes, followed by de-lumping using a 10-mesh screen. Next, half of the magnesium stearate was added and blended for 5 minutes The blend was then roller compacted with a Vector TF mini roller compactor, fitted with “S”-type rolls, using an auger speed of 30 rpm, a roller speed of 4 rpm, and a roller pressure of 30 Kgf/cm². The resulting compacted material was then milled using a Fitzpatrick M5A mill at a power setting of 350 rpm, with a sieve size of 16 mesh. The second half of the magnesium stearate was added next, and the material was blended for 5 minutes in a V-blender. This material was then formed into 800 mg tablets using 1/2-inch SRC tooling on a Killian T-100 (feeder frame speed 30 rpm, 30,000 tablets/hour), and compressed to a hardness of 25 Kp.

The tablets above were coated in a Freund HCT-30 pan-coater using an aqueous solution of 3.5 wt % Opadry® II White and 0.5 wt % Opadry® II Clear. The following coating conditions were used: tablet bed weight, 1000 g; pan speed, 17 rpm; outlet temperature, 42° C.; and solution flow, 6 g/min. Average disintegration time in deionized water was <5 minutes.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

1. A pharmaceutical composition comprising a glycogen phosphorylase inhibitor and a concentration-enhancing polymer wherein a portion of said glycogen phosphorylase inhibitor binds to a portion or all portions of the following residues of a glycogen phosphorylase enzyme:

| parent | |
|---|---|
| secondary | |
| structure | residue number |
| | 13-23 |
| helix α1 | 24-37 |
| turn | 38-39, 43, 46-47 |
| helix α2 | 48-66, 69-70, 73-74, 76-78 |
| | 79-80 |
| strand β1 | 81-86 |
| | 87-88 |
| strand β2 | 89-92 |
| | 93 |
| helix α3 | 94-102 |
| | 103 |
| helix α4 | 104-115 |
| | 116-117 |
| helix α5 | 118-124 |
| | 125-128 |
| strand β3 | 129-131 |
| | 132-133 |
| helix α6 | 134-150 |
| | 151-152 |
| strand β4 | 153-160 |
| | 161 |
| strand β4b | 162-163 |
| | 164-166 |
| strand β5 | 167-171 |
| | 172-173 |
| strand β6 | 174-178 |
| | 179-190 |
| strand β7 | 191-192 |
| | 194, 197 |
| strand β8 | 198-209 |
| | 210-211 |
| strand β9 | 212-216 |
| strand β10 | 219-226, 228-232 |
| | 233-236 |
| strand β11 | 237-239, 241, 243-247 |
| | 248-260 |
| helix α7 | 261-276 |
| strand β11b | 277-281 |
| reverse turn | 282-289 |
| helix α8 | 290-304. |

2. A pharmaceutical composition comprising a glycogen phosphorylase inhibitor and a concentration-enhancing polymer, said glycogen phosphorylase inhibitor being selected from the group consisting of Formula I, Formula II, Formula III and Formula IV; wherein Formula I is 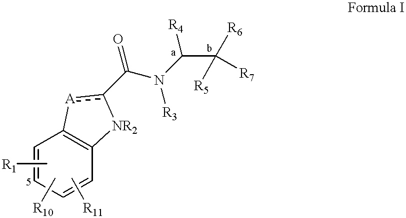
or the pharmaceutically acceptable salts or prodrugs thereof wherein the dotted line (- - -) is an optional bond wherein;
A is —C(H)═, —C((C₁-C₄)alkyl)═ or —C(halo)═ when the dotted line (- - -) is a bond, or A is methylene or —CH((C₁-C₄)alkyl)— when the dotted line (- - -) is not a bond;
R₁, R₁₀ or R₁₁ are each independently H, halo, 4-, 6- or 7-nitro, cyano, (C₁-C₄)alkyl, (C₁-C₄)alkoxy, fluoromethyl, difluoromethyl or trifluoromethyl;
R₂ is H;
R₃ is H or (C₁-C₅)alkyl;
R₄ is H, methyl, ethyl, n-propyl, hydroxy(C₁-C₃)alkyl, (C₁-C₃)alkoxy(C₁-C₃)alkyl, phenyl(C₁-C₄)alkyl, phenylhydroxy(C₁-C₄)alkyl, phenyl(C₁-C₄)alkoxy(C₁-C₄)alkyl, thien-2- or -3-yl(C₁-C₄)alkyl or fur-2- or -3-yl(C₁-C₄)alkyl wherein said R₄ rings are mono-, di- or tri-substitutted independently on carbon with H, halo, (C₁-C₄)alkyl, (C₁-C₄)alkoxy, trifuloromethyl, hydroxy, amino or cyano; or
R₄ is pyrid-2-, -3- or -4-yl(C₁-C₄)alkyl, thiazol-2-, -4- or -5-yl(C₁-C₄)alkyl, imidazol-1-, -2-, -4- or -5-yl(C₁-C₄)alkyl, pyrrol-2- or -3-yl(C₁-C₄)alkyl, oxazol-2-, -4- or -5-yl(C₁-C₄)alkyl, pyrazol-3-, -4- or -5-yl(C₁-C₄)alkyl, isoxazol-3-, -4-, -5-yl(C₁-C₄)alkyl, isothiazol-3-, -4-, -5-yl(C₁-C₄)alkyl, pyridazin-3- or -4-yl-(C₁-C₄)alkyl, pyrimidin-2-, -4-, -5- or -6-yl(C₁-C₄)alkyl, pyrazin-2- or -3-yl(C₁-C₄)alkyl or 1,3,5-triazin-2-yl (C₁-C₄)alkyl, wherein said preceding R₄ heterocycles are optionally mono- or di-substituted independently with halo, trifluoromethyl, (C₁-C₄)alkyl, (C₁-C₄)alkoxy, amino or hydroxy and said mono- or di-substituents are bonded to carbon;
R₅ is H, hydroxy, fluoro, (C₁-C₅)alkyl, (C₁-C₅)alkoxy, (C₁-C₆)alkanoyl, amino(C₁-C₄)alkoxy, mono-N- or di-N,N-(C₁-C₄)alkylamino(C₁-C₄)alkoxy, carboxy(C₁-C₄)alkoxy, (C₁-C₅)alkoxy-carbonyl(C₁-C₄)alkoxy, benzyloxycarbonyl(C₁-C₄)alkoxy, or carbonyloxy wherein said carbonyloxy is carbon-carbon linked with phenyl, thiazolyl, imidazolyl, 1H-indolyl, furyl, pyrrolyl, oxazolyl, pyrazolyl, isoxazolyl, isothiazolyl, pyridazinyl, pyrimidinyl, pyrazinyl or 1,3,5-trizinyl and wherein said preceding R₅₈ rings are optionally monosubstituted with halo, (C₁-C₄)alkyl, (C₁-C₄)alkoxy, hydroxy, amino or trifluoromethyl and said mono-substituents are bonded to carbon;
R₇ is H, fluoro or (C₁-C₅)alkyl; or
R₅ and R₇ taken together are oxo;
R₆ is carboxy or (C₁-C₈)alkoxycarbonyl, C(O)NR₈R₉ or C(O)R₁₂ wherein
R₈ is H, (C₁-C₃)alkyl, hydroxy or (C₁-C₃)alkoxy; and
R₉ is H, (C₁-C₈)alkyl, hydroxy, (C₁-C₈)alkoxy, methylene-perfluorinated(C₁-C₈)alkyl, phenyl, pyridyl, thienyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, piperidinyl, morpholinyl, pyridazinyl, pyrimidinyl, pyrazinyl, piperazinyl or 1,3,5-triazinyl wherein said preceding R₉ rings are carbon-nitrogen linked; or
R₉ is mono-, di- or tri-substituted (C₁-C₅)alkyl, wherein said substituents are independently H, hydroxy, amino, mono-N- or di-N,N-(C₁-C₅)alkylamino; or
R₉ is mono- or di-substituted (C₁-C₅)alkyl, wherein said substituents are independently phenyl, pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, pyridinyl, piperidinyl, morpholinyl, pyridazinyl, pyrimidinyl, pyrazinyl, piperazinyl or 1,3,5-triazinyl
wherein the nonaromatic nitrogen-containing R₉ rings are optionally mono-substituted on nitrogen with (C₁-C₆)alkyl, benzyl, benzoyl or (C₁-C₆)alkoxycarbonyl and wherein the R₉ rings are optionally mono-substituted on carbon with halo, (C₁-C₄)alkyl, (C₁-C₄)alkoxy, hydroxy, amino, or mono-N- and di-N,N (C₁-C₅)alkylamino provided that no quaternized nitrogen is included and there are no nitrogen-oxygen, nitrogen-nitrogen or nitrogen-halo bonds;
R₁₂ is piperazin-1-yl, 4-(C₁-C₄)alkylpiperazin-1-yl, 4-formylpiperazin-1-yl, morpholino, thiomorpholino, 1-oxothiomorpholino, 1,1-dioxo-thiomorpholino, thiazolidin-3-yl, 1-oxo-thiazolidin-3-yl, 1,1-dioxo-thiazolidin-3-yl, 2-(C₁-C₆)alkoxycarbonylpyrrolidin-1-yl, oxazolidin-3-yl or 2(R)-hydroxymethylpyrrolidin-1-yl; or
R₁₂ is 3- and/or 4-mono- or di-substituted oxazetidin-2-yl, 2-, 4-, and/or 5-mono- or di-substituted oxazolidin-3-yl, 2-, 4-, and/or 5-mono- or di-substituted thiazolidin-3-yl, 2-, 4- and/or 5-mono- or di-substituted 1-oxothiazolidin-3-yl, 2-, 4-, and/or 5-mono- or di-substituted 1,1-dioxothiazolidin-3-yl, 3- and/or 4-mono- or di-substituted pyrrolidin-1-yl, 3-, 4- and/or 5-, mono-, di- or tri-substituted piperidin-1-yl, 3-, 4-, and/or 5-mono-, di-, or tri-substituted piperazin-1-yl, 3-substituted azetidin-1-yl, 4- and/or 5-, mono- or di-substituted 1,2-oxazinan-2-yl, 3- and/or 4-mono- or di-substituted pyrazolidin-1-yl, 4- and/or 5-, mono- or di-substituted isoxazolidin-2-yl, 4- and/or 5-, mono- and/or di-substituted isothiazolidin-2-yl wherein said R₁₂ substituents are independently H, halo, (C₁-C₅)alkyl, hydroxy, amino, mono-N- or di-N,N-(C₁-C₅)alkylamino, formyl, oxo, hydroxyimino, (C₁-C₅)alkoxy, carboxy, carbamoyl, mono-N-or di-N,N-(C₁-C₅)alkylcarbamoyl, (C₁-C₄)alkoxyimino, (C₁-C₄)alkoxymethoxy, (C₁-C₆)alkoxycarbonyl, carboxy(C₁-C₅)alkyl or hydroxy (C₁-C₅) alkyl;
with the proviso that if R₄ is H, methyl, ethyl or n-propyl, R₅ is OH;
with the proviso that if R₅ and R₇ are H, then R₄ is not H, methyl, ethyl, n-propyl, hydroxy(C₁-C₃)alkyl or (C₁-C₃)alkoxy(C₁-C₃)alkyl and R₆ is C(O)NR₈R₉, C(O)R₁₂ or (C₁-C₄) alkoxycarbonyl;
and wherein Formula II is 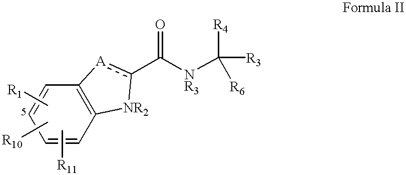
or the pharmaceutically acceptable salts or prodrugs thereof wherein the dotted line (- - -) is an optional bond wherein
A is —C(H), —C((C₁-C₄)alkyl)═, —C(halo)═ or —N═, when the dotted line (- - -) is a bond, or A is methylene or —CH((C₁-C₄)alkyl)—, when the dotted line (- - -) is not a bond;
R₁, R₁₀ or R₁₁ are each independently H, halo, cyano, 4-, 6- or 7-nitro, (C₁-C₄)alkyl, (C₁-C₄)alkoxy, fluoromethyl, difluoromethyl or trifluoromethyl;
R₂ is H;
R₃ is H or (C₁-C₅)alkyl;
R₄ is H, methyl, ethyl, n-propyl, hydroxy(C₁-C₃)alkyl, (C₁-C₃)alkoxy(C₁-C₃)alkyl, phenyl (C₁-C₄)alkyl, phenylhydroxy(C₁-C₄)alkyl, (phenyl)((C₁-C₄)-alkoxy)(C₁-C₄)alkyl, thien-2- or -3-yl(C₁-C₄)alkyl or fur-2- or -3-yl(C₁-C₄)alkyl wherein said R₄ rings are mono-, di- or tri-substituted independently on carbon with H, halo, (C₁-C₄)alkyl, (C₁-C₄)alkoxy, trifuloromethyl, hydroxy, amino, cyano or 4,5-dihydro-1H-imidazol-2-yl; or
R₄ is pyrid-2-, -3- or -4-yl(C₁-C₄)alkyl, thiazol-2-, -4- or -5-yl(C₁-C₄)alkyl, imidazol-2-, -4-, or -5-yl(C₁-C₄)alkyl, pyrrol-2- or -3-yl(C₁-C₄)alkyl, oxazol-2-, -4- or -5-yl(C₁-C₄)alkyl, pyrazol-3-, -4- or -5-yl(C₁-C₄)alkyl, isoxazol-3-, -4- or -5-yl(C₁-C₄)alkyl, isothiazol-3-, -4- or -5-yl(C₁-C₄)alkyl, pyridazin-3- or -4-yl(C₁-C₄)alkyl, pyrimidin-2-, -4-, -5- or -6-yl(C₁-C₄)alkyl, pyrazin-2- or -3-yl(C₁-C₄)alkyl, 1,3,5-triazin-2-yl(C₁-C₄)alkyl or indol-2-(C₁-C₄)alkyl, wherein said preceding R₄ heterocycles are optionally mono- or di-substituted independently with halo, trifluoromethyl, (C₁-C₄)alkyl, (C₁-C₄)alkoxy, amino, hydroxy or cyano and said substituents are bonded to carbon; or
R₄ is R₁₅-carbonyloxymethyl, wherein said R₁₅ is phenyl, thiazolyl, imidazolyl, 1H-indolyl, furyl, pyrrolyl, oxazolyl, pyrazolyl, isoxazolyl, isothiazolyl, pyridyl, pyridazinyl, pyrimidinyl, pyrazinyl or 1,3,5-triazinyl and wherein said preceding R₁₅ rings are optionally mono- or di-substituted independently with halo, amino, hydroxy, (C₁-C₄)alkyl, (C₁-C₄)alkoxy or trifluoromethyl and said mono- or di-substituents are bonded to carbon;
R₅ is H, methyl, ethyl, n-propyl, hydroxymethyl or hydroxyethyl;
R₆ is carboxy, (C₁-C₈)alkoxycarbonyl, benzyloxycarbonyl, C(O)NR₈R₉ or C(O)R₁₂
wherein R₈ is H, (C₁-C₆)alkyl, cyclo (C₃-C₆)alkyl, cyclo(C₃-C₆)alkyl(C₁-C₅)alkyl, hydroxy or (C₁-C₈)alkoxy; and
R₉ is H, cyclo(C₃-C₈)alkyl, cyclo(C₃-C₈)alkyl (C₁-C₅)alkyl, cyclo(C₄-C₇)alkenyl, cyclo(C₃-C₇)alkyl(C₁-C₅)alkoxy, cyclo(C₃-C₇)alkyloxy, hydroxy, methylene-perfluorinated (C₁-C₈)alkyl, phenyl, or a heterocycle wherein said heterocycle is pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, pyridinyl, piperidinyl, morpholinyl, pyridazinyl, pyrimidinyl, pyrazinyl, piperazinyl, 1,3,5-triazinyl, benzothiazolyl, benzoxazolyl, benzimidazolyl, thiochromanyl or tetrahydrobenzothiazolyl wherein said heterocycle rings are carbon-nitrogen linked; or
R₉ is (C₁-C₆)alkyl or (C₁-C₈)alkoxy wherein said (C₁-C₆)alkyl or (C₁-C₈)alkoxy is optionally monosubstituted with cyclo(C₄-C₇)alken-1-yl, phenyl, thienyl, pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, piperidinyl, morpholinyl, thiomorpholinyl, 1-oxothiomorpholinyl, 1,1-dioxothiomorpholinyl, pyridazinyl, pyrimidinyl, pyrazinyl, piperazinyl, 1,3,5-triazinyl or indolyl and wherein said (C₁-C₆)alkyl or (C₁-C₈)alkoxy are optionally additionally independently mono- or di-substituted with halo, hydroxy, (C₁-C₅)alkoxy, amino, mono-N- or di-N,N-(C₁-C₅)alkylamino, cyano, carboxy, or (C₁-C₄) alkoxycarbonyl; and
wherein the R₉ rings are optionally mono- or di-substituted independently on carbon with halo, (C₁-C₄)alkyl, (C₁-C₄)alkoxy, hydroxy, hydroxy(C₁-C₄)alkyl, amino(C₁-C₄)alkyl, mono-N- or di-N,N-(C₁-C₄)alkylamino (C₁-C₄)alkyl, (C₁-C₄)alkoxy(C₁-C₄)alkyl, amino, mono-N- or di-N,N-(C₁-C₄)alkylamino, cyano, carboxy, (C₁-C₅)alkoxycarbonyl, carbamoyl, formyl or trifluoromethyl and said R₉ rings may optionally be additionally mono- or di-substituted independently with (C₁-C₅)alkyl or halo;
with the proviso that no quaternized nitrogen on any R₉ heterocycle is included;
R₁₂ is morpholino, thiomorpholino, 1-oxothiomorpholino, 1,1-dioxothiomorpholino, thiazolidin-3-yl, 1-oxothiazolidin-3-yl, 1,1-dioxothiazolidin-3-yl, pyrrolidin-1-yl, piperidin-1-yl, piperazin-1-yl, piperazin-4-yl, azetidin-1-yl, 1,2-oxazinan-2-yl, pyrazolidin-1-yl, isoxazolidin-2-yl, isothiazolidin-2-yl, 1,2-oxazetidin-2-yl, oxazolidin-3-yl, 3,4-dihydroisoquinolin-2-yl, 1,3-dihydroisoindol-2-yl, 3,4-dihydro-2H-quinol-1-yl, 2,3-dihydro-benzo[1,4]oxazin-4-yl, 2,3-dihydro-benzo[1,4]-thiazine-4-yl, 3,4-dihydro-2H-quinoxalin-1-yl, 3,4-dihydro-benzo[c][1,2]oxazin-1-yl, 1,4-dihydro-benzo[d][1,2]oxazin-3-yl, 3,4-dihydro-benzo[e][1,2]-oxazin-2-yl, 3H-benzo[d]isoxazol-2-yl, 3H-benzo[c]isoxazol-1-yl or azepan-1-yl,
wherein said R₁₂ rings are optionally mono-, di- or tri-substituted independently with halo, (C₁-C₅)alkyl, (C₁-C₅)alkoxy, hydroxy, amino, mono-N- or di-N,N-(C₁-C₅)alkylamino, formyl, carboxy, carbamoyl, mono-N- or di-N,N-(C₁-C₅)alkylcarbamoyl, (C₁-C₆)alkoxy (C₁-C₃)alkoxy, (C₁-C₅)alkoxycarbonyl, benzyloxycarbonyl, (C₁-C₅)alkoxycarbonyl (C₁-C₅)alkyl, (C₁-C₄)alkoxycarbonylamino, carboxy(C₁-C₅)alkyl, carbamoyl(C₁-C₅)alkyl, mono-N- or di-N,N-(C₁-C₄) alkylcarbamoyl (C₁-C₅) alkyl, hydroxy(C₁-C₅)alkyl, (C₁-C₄)alkoxy(C-C₄)alkyl, amino(C₁-C₄)alkyl, mono-N- or di-N,N-(C₁-C₄)alkylamino(C₁-C₄)alkyl, oxo, hydroxyimino or (C₁-C₆)alkoxyimino and wherein no more than two substituents are selected from oxo, hydroxyimino or (C₁-C₆)alkoxyimino and oxo, hydroxyimino or (C₁-C₆)alkoxyimino are on nonaromatic carbon; and
wherein said R₁₂ rings are optionally additionally mono- or di-substituted independently with (C₁-C₅)alkyl or halo;
with the proviso that when R₆ is (C₁-C₅)alkoxycarbonyl or benzyloxycarbonyl then R₁ is 5-halo, 5-(C₁-C₄)alkyl or 5-cyano and R₄ is (phenyl) (hydroxy), (C₁-C₄)alkyl, (phenyl) ((C₁-C₄)alkoxy) (C₁-C₄)alkyl, hydroxymethyl or Ar(C₁-C₂)alkyl, wherein Ar is thien-2- or -3-yl, fur-2- or -3-yl or phenyl wherein said Ar is optionally mono- or di-substituted independently with halo; with the provisos that when R₄ is benzyl and R₅ is methyl, R₁₂ is not 4-hydroxy-piperidin-1-yl or when R₄ is benzyl and R₅ is methyl R₆ is not C(O)N(CH₃)₂;
with the proviso that when R₁ and R₁₀ and R₁₁ are H, R₄ is not imidazol-4-ylmethyl, 2-phenylethyl or 2-hydroxy-2-phenylethyl;
with the proviso that when R₈ and R₉ are n-pentyl, R₁ is 5-chloro, 5-bromo, 5-cyano, 5(C₁-C₅)alkyl, 5(C₁-C₅)alkoxy or trifluoromethyl;
with the proviso that when R₁₂ is 3,4-dihydroisoquinol-2-yl, said 3,4-dihydroisoquinol-2-yl is not substituted with carboxy((C₁-C₄)alkyl;
with the proviso that when R₈ is H and R₉ is (C₁-C₆)alkyl, R₉ is not substituted with carboxy or (C₁-C₄)alkoxycarbonyl on the carbon which is attached to the nitrogen atom N of NHR₉; and
with the proviso that when R₆ is carboxy and R₁, R₁₀, R₁₁ and R₅ are all H, then R₄ is not benzyl, H, (phenyl)(hydroxy)methyl, methyl, ethyl or n-propyl;
and wherein Formula III is 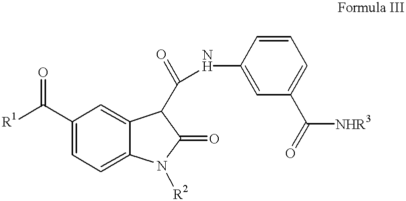
or a prodrug thereof or a pharmaceutically acceptable salt of said compound or said prodrug wherein
R¹ is (C₁-C₄)alkyl, (C₁-C₇)cycloalkyl, phenyl or phenyl sbustituted with up to three (C₁-C₄)alkyl, (C₁-C₄) alkoxy or halogen;
R² is (C₁-C₄) alkyl; and
R³ is (C₃-C₇)cycloalkyl; phenyl; phenyl substituted at the para position with (C₁-C₄) alkyl, halo, hydroxy (C₁-C₄) alkyl or trifluoromethyl; phenyl substituted at the meta position with fluoro; or phenyl substituted at the ortho position with fluoro;
and wherein Formula IV is 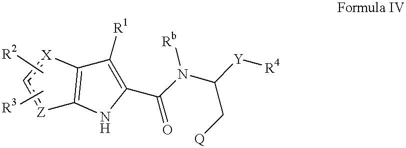
a stereoisomer, pharmaceutically acceptable salt or prodrug thereof, or a pharmaceutically acceptable salt of the prodrug, wherein
Q is aryl, substitued aryl, heteroaryl, or substitued heteroaryl;
each Z and X are independently (C, CH or CH₂), N, O or S;
X¹ is NR^{a}, —CH₂—, O or S;
each - - - - is independently a bond or is absent, provided that both - - - - are not simlutaneously bonds;
R¹ is hydrogen, halogen, —OC₁—C₈alkyl, —SC₁₋—C₈alkyl, —C₁-C₈alkyl, —CF₃, —NH₂, —NHC₁—C₈alkyl, —N(C₁-C₈alkyl)₂, —NO₂, —CN, —CO₂H, —CO₂C₁-C₈alkyl, —C₂-C₈alkenyl, or —C₂-C₈alkynyl;
each R^{a} and R^{b} is independently hydrogen or —C₁-C₈alkyl; 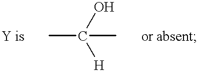
R² and R³ are independently hydrogen, halogen, —C₁-C₈alkyl, —CN, —C≡C—Si(CH₃)₃, —OC₁-C₈alkyl, —SC₁-C₈alkyl, —CF₃, —NH₂, —NHC₁-C₈alkyl, —N(C₁-C₈alkyl)₂, —NO₂, —CO₂H, —CO₂C₁-C₈alkyl, —C₂-C₈alkenyl, or —C₂-C₈alkynyl, or R² and R³ together with the atoms on the ring to which they are attached form a five or six membered ring containing from 0 to 3 heteroatoms and from 0 to 2 double bonds;
R⁴is —C(═O)—A;
A is —NR^{a}R^{d}, —NR^{a}CH₂CH₂OR^{a}, [chemical structure]
each R^{d} is independently hydrogen, C₁-C₈alkyl, C₁-C₈alkoxy, aryl, substituted aryl, heteroaryl, or substituted heteroaryl;
each R^{c} is independently hydrogen, —C(═O)OR^{a}, —OR^{a}, —SR^{a}, or —NR^{a}R^{a}; and each n is independently 1-3.

3. A pharmaceutical composition comprising a glycogen phosphorylase inhibitor and a concentration-enhancing polymer, said glycogen phosphorylase inhibitor having a solubility in aqueous solution, in the absence of said concentration-enhancing polymer, of less than 1 mg/ml at any pH of from 1 to 8.

4. The composition of any one of claims 1-3 wherein said composition is a solid amorphous dispersion.

5. The composition of claim 4 wherein said dispersion is substantially homogeneous.

6. The composition of claim 4 wherein said glycogen phosphorylase inhibitor is almost completely amorphous.

7. The composition of any one of claims 1-3 wherein said composition is a simple physical mixture.

8. The composition of claim 7 wherein said mixture is substantially homogeneous.

9. The composition of claim 7 wherein said glycogen phosphorylase inhibitor is almost completely amorphous.

10. The composition of any one of claims 1-3 wherein said glycogen phosphorylase inhibitor is in a solid amorphous dispersion and only a portion of said concentration-enhancing polymer is present in said dispersion.

11. The composition of claim 1 wherein a portion of said glycogen phosphorylase inhibitor binds to one or more of the following residues of said glycogen phosphorylase enzyme in one or both subunits:

| parent | |
|---|---|
| secondary | |
| structure | residue number |
| | 13-23 |
| helix α1 | 24-37 |
| turn | 38-39, 43, 46-47 |
| helix α2 | 48-66, 69-70, 73-74, 76-78 |
| | 79-80 |
| strand β2 | 91-92 |
| | 93 |
| helix α3 | 94-102 |
| | 103 |
| helix α4 | 104-115 |
| | 116-117 |
| helix α5 | 118-124 |
| | 125-128 |
| strand β3 | 129-130 |
| strand β4 | 159-160 |
| | 161 |
| strand β4b | 162-163 |
| | 164-166 |
| strand β5 | 167-168 |
| strand β6 | 178 |
| | 179-190 |
| strand β7 | 191-192 |
| | 194, 197 |
| strand β9 | 198-200 |
| strand β10 | 220-226 |
| | 228-232 |
| | 233-236 |
| strand β11 | 237-239, 241, 243-247 |
| | 248-260 |
| helix α7 | 261-276 |
| strand β11b | 277-280 |

12. The composition of claim 1 wherein a portion of said glycogen phosphorylase inhibitor binds to a portion or all portions of the following residues of said glycogen phosphorylase enzyme in one or both subunits:
residue number
33-39
49-66
94
98
102
125-126
160
162
182-192
197
224-226
228-231
238-239
241
245
247

13. The composition of claim 1 wherein a portion of said glycogen phosphorylase inhibitor binds to a portion or all portions of the following residues of said glycogen phosphorylase enzyme in one or both subunits:
residue number
37-39
53
57
60
63-64
184-192
226
229

14. The composition of any one of claims 2-3 wherein a portion of said glycogen phosphorylase inhibitor binds to a portion or all portions of the following residues of a glycogen phosphorylase enzyme:

| parent | |
|---|---|
| secondary | |
| structure | residue number |
| | 13-23 |
| helix α1 | 24-37 |
| turn | 38-39, 43, 46-47 |
| helix α2 | 48-66, 69-70, 73-74, 76-78 |
| | 79-80 |
| strand β1 | 81-86 |
| | 87-88 |
| strand β2 | 89-92 |
| | 93 |
| helix α3 | 94-102 |
| | 103 |
| helix α4 | 104-115 |
| | 116-117 |
| helix α5 | 118-124 |
| | 125-128 |
| strand β3 | 129-131 |
| | 132-133 |
| helix α6 | 134-150 |
| | 151-152 |
| strand β4 | 153-160 |
| | 161 |
| strand β4b | 162-163 |
| | 164-166 |
| strand β5 | 167-171 |
| | 172-173 |
| strand β6 | 174-178 |
| | 179-190 |
| strand β7 | 191-192 |
| | 194, 197 |
| strand β8 | 198-209 |
| | 210-211 |
| strand β9 | 212-216 |
| strand β10 | 219-226, 228-232 |
| | 233-236 |
| strand β11 | 237-239, 241, 243-247 |
| | 248-260 |
| helix α7 | 261-276 |
| strand β11b | 277-281 |
| reverse turn | 282-289 |
| helix α8 | 290-304 |

15. The composition of claim 14 wherein a portion of said glycogen phosphorylase inhibitor binds to a portion or all portions of the following residues of said glycogen phosphorylase enzyme in one or both subunits:

| parent secondary | |
|---|---|
| structure | residue number |
| | 13-23 |
| helix α1 | 24-37 |
| turn | 38-39, 43, 46-47 |
| helix α2 | 48-66, 69-70, 73-74, 76-78 |
| | 79-80 |
| strand β2 | 91-92 |
| | 93 |
| helix α3 | 94-102 |
| | 103 |
| helix α4 | 104-115 |
| | 116-117 |
| helix α5 | 118-124 |
| | 125-128 |
| strand β3 | 129-130 |
| strand β4 | 159-160 |
| | 161 |
| strand β4b | 162-163 |
| | 164-166 |
| strand β5 | 167-168 |
| strand β6 | 178 |
| | 179-190 |
| strand β7 | 191-192 |
| | 194, 197 |
| strand β9 | 198-200 |
| strand β10 | 220-226 |
| | 228-232 |
| | 233-236 |
| strand β11 | 237-239, 241, 243-247 |
| | 248-260 |
| helix α7 | 261-276 |
| strand β11b | 277-280 |

16. The composition of claim 14 wherein a portion of said glycogen phosphorylase inhibitor binds to a portion or all portions of the following residues of said glycogen phosphorylase enzyme in one or both subunits:
residue number
33-39
49-66
94
98
102
125-126
160
162
182-192
197
224-226
228-231
238-239
241
245
247

17. The composition of claim 14 wherein a portion of said glycogen phosphorylase inhibitor binds to a portion or all portions of the following residues of said glycogen phosphorylase enzyme in one or both subunits:
residue number
37-39
53
57
60
63-64
184-192
226
229

18. The composition of claim 1 wherein said glycogen phosphorylase inhibitor has the structure of Formula I defined in claim 2.

19. The composition of claim 2 wherein said glycogen phosphorylase inhibitor is selected from the group consisting of 5-chloro-1H-indole-2-carboxylic acid [(1S)-((R)-hydroxy-dimethylcarbamoylmethyl)-2-phenyl-ethyl]-amide, 5-chloro-1H-indole-2-carboxylic acid [(1S)-((R)-hydroxy-methoxy-methylcarbamoylmethyl)-2-phenyl-ethyl]-amide, 5-chloro-1H-indole-2-carboxylic acid [(1S)-benzyl-(2R)-hydroxy-3-((3S)-hydroxy-pyrrolidin-1-yl)-3-oxo-propyl]-amide, 5-chloro-1H-indole-2-carboxylic acid [(1S)-benzyl-(2R)-hydroxy-3-((3R,4S)-dihydroxy-pyrrolidin-1-yl)-3-oxo-propyl]-amide, 5-chloro-1H-indole-2-carboxylic acid [(1S)-benzyl-(2R)-hydroxy-3-((3R,4R)-dihydroxy-pyrrolidin-1-yl)-3-oxo-propyl]-amide, and 5-chloro-1H-indole-2-carboxylic acid [(1S)-benzyl-(2R)-hydroxy-3-morpholin-4-yl-3-oxo-propyl]-amide.

20. The composition of claim 18 wherein said glycogen phosphorylase inhibitor is selected from the group consisting of 5-chloro-1H-indole-2-carboxylic acid [(1S)-((R)-hydroxy-dimethylcarbamoylmethyl)-2-phenyl-ethyl]-amide, 5-chloro-1H-indole-2-carboxylic acid [(1S)-((R)-hydroxy-methoxy-methylcarbamoylmethyl)-2-phenyl-ethyl]-amide, 5-chloro-1H-indole-2-carboxylic acid [(1S)-benzyl-(2R)-hydroxy-3-((3S)-hydroxy-pyrrolidin-1-yl)-3-oxo-propyl]-amide, 5-chloro-1H-indole-2-carboxylic acid [(1S)-benzyl-(2R)-hydroxy-3-((3R,4S)-dihydroxy-pyrrolidin-1-yl)-3-oxo-propyl]-amide, 5-chloro-1H-indole-2-carboxylic acid [(1S)-benzyl-(2R)-hydroxy-3-((3R,4R)-dihydroxy-pyrrolidin-1-yl)-3-oxo-propyl]-amide, and 5-chloro-1H-indole-2-carboxylic acid [(1S)-benzyl-(2R)-hydroxy-3-morpholin-4-yl-3-oxo-propyl]-amide.

21. The composition of claim 1 wherein said glycogen phosphorylase inhibitor has the structure of Formula II defined in claim 2.

22. The composition of claim 2 wherein said glycogen phosphorylase inhibitor is selected from the group consisting of 5-chloro-1H-indole-2-carboxylic acid [2-((3R,4S)-3,4-dihydroxy-pyrrolidin-1-yl)-2-oxo-ethyl]-amide 5-chloro-1H-indole-2-carboxylic acid [(1S)-benzyl-2-((3S,4S)-3,4-dihydroxy-pyrrolidin-1-yl)-2-oxo-ethyl]-amide, 5-chloro-1H-indole-2-carboxylic acid [(1S)-benzyl-2-((3R,4S)-3,4-dihydroxy-pyrrolidin-1-yl)-2-oxo-ethyl]-amide, 5-chloro-1H-indole-2-carboxylic acid [(1S)-(4-fluoro-benzyl)-2-(4-hydroxy-piperidin-1-yl)-2-oxo-ethyl]-amide, 5-chloro-1H-indole-2-carboxylic acid [(1S)-benzyl-2-(3-hydroxy-azetidin-1-yl)-2-oxo-ethyl]-amide, 5-chloro-1H-indole-2-carboxylic acid [2-(1,1-dioxo-thiazolidin-3-yl)-2-oxo-ethyl]-amide, and 5-chloro-1H-indole-2-carboxylic acid [2-(1-oxo-thiazolidin-3-yl)-2-oxo-ethyl]-amide.

23. The composition of claim 21 wherein said glycogen phosphorylase inhibitor is selected from the group consisting of 5-chloro-1H-indole-2-carboxylic acid [2-((3R,4S)-3,4-dihydroxy-pyrrolidin-1-yl)-2-oxo-ethyl]-amide, 5-chloro-1H-indole-2-carboxylic acid [(1S)-benzyl-2-((3S,4S)-3,4-dihydroxy-pyrrolidin-1-yl)-2-oxo-ethyl]-amide, 5-chloro-1H-indole-2-carboxylic acid [(1S)-benzyl-2-((3R,4S)-3,4-dihydroxy-pyrrolidin-1-yl)-2-oxo-ethyl]-amide, 5-chloro-1H-indole-2-carboxylic acid [(1S)-(4-fluoro-benzyl)-2-(4-hydroxy-piperidin-1-yl)-2-oxo-ethyl]-amide, 5-chloro-1H-indole-2-carboxylic acid [(1S)-benzyl-2-(3-hydroxy-azetidin-1-yl)-2-oxo-ethyl]-amide, 5-chloro-1H-indole-2-carboxylic acid [2-(1,1-dioxo-thiazolidin-3-yl)-2-oxo-ethyl]-amide, and 5-chloro-1H-indole-2-carboxylic acid [2-(1-oxo-thiazolidin-3-yl)-2-oxo-ethyl]-amide.

24. The composition of claim 1 wherein said glycogen phosphorylase inhibitor has the structure of Formula III as defined in claim 2.

25. The composition of claim 2 wherein said glycogen phosphorylase inhibitor is selected from the group consisting of 5-acetyl-1-ethyl-2,3-dihydro-2-oxo-N-[3-[(phenylamino)carbonyl]phenyl]]-1H-Indole-3-carboxamide, 5-acetyl-N-[3-[(cyclohexylamino)carbonyl]phenyl-1-ethyl-2,3-dihydro-2-oxo-1H-Indole-3-carboxamide, and 5-acetyl-N-[3-[[(4-bromophenyl)amino]carbonyl]phenyl]-2,3-dihydro-1-methyl-2-oxo-1H-Indole-3-carboxamide.

26. The composition of claim 24 wherein said glycogen phosphorylase inhibitor is selected from the group consisting of 5-acetyl-1-ethyl-2,3-dihydro-2-oxo-N-[3-[(phenylamino)carbonyl]phenyl]-1H-Indole-3-carboxamide, 5-acetyl-N-[3-[(cyclohexylamino)carbonyl]phenyl-1-ethyl-2,3-dihydro-2-oxo-1H-Indole-3-carboxamide, and 5-acetyl-N-[3-[[(4-bromophenyl)amino]carbonyl]phenyl]-2,3-dihydro-1-methyl-2-oxo-1H-Indole-3-carboxamide.

27. The composition of claim 1 wherein said glycogen phosphorylase inhibitor has the structure of Formula IV as defined in claim 2.

28. The composition of claim 2 wherein said glycogen phosphorylase inhibitor is selected from the group consisting of 2-Chloro-6H-thieno[2,3-b]pyrrole-5-carboxylic acid [(1S)-benzyl-2-((3R,4S)-dihydroxy-pyrrolidin-1-yl)-2-oxo-ethyl]-amide, and 2-chloro-6H-thieno[2,3-b]pyrrole-5-carboxylic acid [(1S)-benzyl-(2R)-hydroxy-3-((3R,4S)-dihydroxy-pyrrolidin-1-yl)-3-oxo-propyl]-amide.

29. The composition of claim 27 wherein said glycogen phosphorylase inhibitor is selected from the group consisting of 2-Chloro-6H-thieno[2,3-b]pyrrole-5-carboxylic acid [(1S)-benzyl-2-((3R,4S)-dihydroxy-pyrrolidin-1-yl)-2-oxo-ethyl]-amide, and 2-chloro-6H-thieno[2,3-b]pyrrole-5-carboxylic acid [(1S)-benzyl-(2R)-hydroxy-3-((3R,4S)-dihydroxy-pyrrolidin-1-yl)-3-oxo-propyl]-amide.

30. The composition of any one of claims 1 and 2 wherein said glycogen phosphorylase inhibitor has a solubility in aqueous solution in the absence of said concentration-enhancing polymer of less than 1 mg/ml at any pH of from 1 to 8.

31. The composition of claim 30 wherein said glycogen phosphorylase inhibitor has an aqueous solubility of less than 0.5 mg/ml.

32. The composition of claim 3 wherein said glycogen phosphorylase inhibitor has an aqueous solubility of less than 0.5 mg/ml.

33. The composition of claim 31 wherein said solubility is less than 0.1 mg/mL.

34. The composition of claim 32 wherein said solubility is less than 0.1 mg/mL.

35. The composition of any one of claims 1-3 wherein said glycogen phosphorylase inhibitor has a dose-to-aqueous-solubility ratio of at least 10 ml.

36. The composition of claim 35 wherein said dose-to-aqueous solubility ratio is at least 100 ml.

37. The composition of claim 36 wherein said dose-to-aqueous solubility ratio is at least 400 ml.

38. The composition of any one of claims 1-3 wherein said concentration-enhancing polymer comprises a blend of polymers.

39. The composition of any one of claims 1-3 wherein said concentration-enhancing polymer has at least one hydrophobic portion and at least one hydrophilic portion.

40. The composition of any one of claims 1-3 wherein said concentration-enhancing polymer is an ionizable polymer.

41. The composition of any one of claims 1-3 wherein said concentration-enhancing polymer is selected from the group consisting of ionizable cellulosic polymers, nonionizable cellulosic polymers, and vinyl polymers and copolymers having substituents selected from the group consisting of hydroxyl, alkylacyloxy, and cyclicamido.

42. The composition of any one of claims 1-3 wherein said concentration-enhancing polymer is a cellulosic polymer.

43. The composition of claim 42 wherein said concentration-enhancing polymer is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

44. The composition of claim 42 wherein said concentration-enhancing polymer is selected from the group consisting of hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

45. The composition of claim 42 wherein said concentration-enhancing polymer is selected from the group consisting of cellulose acetate phthalate, methyl cellulose acetate phthalate, ethyl cellulose acetate phthalate, hydroxypropyl cellulose acetate phthalate, hydroxypropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

46. The composition of claim 42 wherein said concentration-enhancing polymer is selected from the group consisting of hydroxypropyl methyl cellulose acetate succinate, cellulose acetate phthalate, hydroxypropyl methyl cellulose phthalate, methyl cellulose acetate phthalate, cellulose acetate trimellitate, hydroxypropyl cellulose acetate phthalate, cellulose acetate terephthalate and cellulose acetate isophthalate.

47. The composition of claim 46 wherein said concentration-enhancing polymer is selected from the group consisting of hydroxypropyl methyl cellulose acetate succinate, hydroxypropyl methyl cellulose phthalate, cellulose acetate phthalate, and cellulose acetate trimellitate.

48. The composition of any one of claims 1-3 wherein said concentration-enhancing polymer is present in an amount sufficient to permit said composition to provide a maximum concentration of said glycogen phosphorylase inhibitor in a use environment that is at least 1.25-fold that of a control composition comprising an equivalent quantity of said glycogen phosphorylase inhibitor and free from said concentration-enhancing polymer.

49. The composition of claim 48 wherein said maximum concentration of said glycogen phosphorylase inhibitor in said use environment is at least 2-fold that of said control composition.

50. The composition of any one of claims 1-3 wherein said composition provides in an aqueous use environment an area under the concentration versus time curve for any period of at least 90 minutes between the time of introduction into the use environment and about 270 minutes following introduction to the use environment that is at least 1.25-fold that of a control composition comprising an equivalent quantity of said glycogen phosphorylase inhibitor and free from said concentration-enhancing polymer.

51. The composition of any one of claims 1-3 wherein said composition provides a relative bioavailability that is at least 1.25 relative to a control composition comprising an equivalent quantity of said glycogen phosphorylase inhibitor and free from said concentration-enhancing polymer.

52. The composition of claim 48 wherein said use environment is in vitro.

53. The composition of claim 48 wherein said use environment is in vivo.

54. The composition of claim 53 wherein said use environment is the gastrointestinal tract of an animal.

55. The composition of claim 54 wherein said animal is a human.

56. The composition of claim 50 wherein said use environment is in vitro.

57. The composition of claim 50 wherein said use environment is in vivo.

58. The composition of claim 57 wherein said use environment is the gastrointentinal tract of an animal.

59. The composition of claim 58 wherein said animal is a human.

60. The composition of claim 4 wherein said dispersion is formed by solvent processing.

61. The composition of claim 60 wherein said solvent processing is spray-drying.

62. A method of treating diabetes, the method comprising the step of administering to a patient having diabetes a therapeutically effective amount of a composition of any one of claims 1-3.

63. The method of claim 62 wherein the diabetes is non-insulin dependent diabetes mellitus (Type 2).

64. The method of claim 62 wherein the diabetes is insulin dependent diabetes mellitus (Type 1).

65. A method of treating or presenting an indication selected from the group consisting of atherosclerosis, diabetic neuropathy, diabetic nephropathy, diabetic retinopathy, cataracts, hypercholesterolemia, hypertriglyceridemia, hyperlipidemia, hyperglycemia, hypertension, tissue ischemia, myocardial ischemia, insulin resistance, bacterial infection, diabetic cardiomyopathy and tumor growth, the method comprising the step of administering to a patient a therapeutically effective amount of a composition of any one of claims 1-3.

66. A method of inhibiting glycogen phosphorylase, the method comprising the step of administering to a patient in need of glycogen phosphorylase inhibition, a glycogen phosphorylase inhibiting amount of a composition of any one of claims 1-3.
